(19) United States
(12) Patent Application Publication
(10) Pub. No.: US 2006/0122374 A1
(43) Pub. Date: Jun. 8, 2006

(54) Albumin-fused anti-angiogenesis peptides

(76) Inventors: Peter Mertins, Marburg (DE); Iihan Celik, Ebsdorfergrund (DE); Oliver Elaker, Ronadorf (DE); Darrell Sleep, Nottingham (GB); Joanna Hay, Devon (GB); Hans-Peter Hanger, Marburg (DE)

Correspondence Address: SYNNESTVEDT & LECHNER, LLP, 2600 ARAMARK TOWER, 1101 MARKET STREET, PHILADELPHIA, PA, 191072950, US

(21) Appl. No.: 10/503,836

(22) PCT Filed: Feb. 7, 2003
(86) PCT No.: PCT/IB03/00433

§ 371 Date: Nov. 2, 2005

Related U.S. Application Data
(60) Provisional application No. 60/355,547, filed on Feb. 7, 2002.

(51) Int. Cl. C07K 14/765; A61K 38/37

(52) U.S. Cl. 530/362; 514/12

(57) ABSTRACT

The invention relates to proteins comprising angiogenesis inhibiting peptides, such as endostatin peptides (including, but not limited to, fragments and variants thereof), which exhibit anti-retroviral activity, fused or conjugated to albumin (including, but not limited to fragments or variants of albumin). These fusion proteins are herein collectively referred to as “albumin fusion proteins of the invention.” These fusion proteins are herein collectively referred to as “albumin fusion proteins of the invention.” These fusion proteins exhibit extended shelf-life and/or extended or therapeutic activity in solution. The invention encompasses therapeutic albumin fusion proteins, compositions, pharmaceutical compositions, formulations and kits. The invention also encompasses nucleic acid molecules encoding the albumin fusion proteins of the invention, as well as vectors containing these nucleic acuds, host cells transformed with these nucleic acids and vectors, and methods of making the albumin fusion proteins of the invention using these nucleic acids, vectors, and/or host cells. The invention also relates to compositions and methods for inhibiting proliferation of vascular endothelial cells and tumor aniogenesis induced cell fusion. The invention further relates to compositions and methods preventing growth of, or promoting regression of, primary tumors and metastases; and for treating cancer, diabetic retinophathy, progressive macular degeneration or rheumatoid arthritis.

### RELATED APPLICATIONS

This application claims priority to U.S. Provisional Application Ser. No. 60/355,547, filed Feb. 7, 2002. The disclosure of that application is incorporated herein by reference in its entirety.

### FIELD OF THE INVENTION

The invention relates to the fields of anti-angiogenesis peptides and albumin fusion proteins.

### BACKGROUND OF THE INVENTION

Angiogenesis, sometimes called neoangiogenesis, is the development of new blood capillaries and vessels.

This process occurs normally in a number of biological situations, including fetal development; menstruation; ovulation; placental development; and the development of collateral blood vessels in areas of disease or ischemia, nerve regeneration, bone growth, and wound healing. All these events, especially fetal development, require the very rapid growth of endothelial cells and their migration and differentiation into a complex network of vessels.

In the normal adult, however, with the exception of the aforementioned biological events (that usually turn on and off within one to two weeks of initiation), angiogenesis is not needed and endothelial cells are quiescent.

Generally, endothelial cells regenerate very slowly, turning over about once every three to four years. The endothelial cells have not lost the ability to divide; rather, they are held in check by a complex balance between endogenous stimulators and inhibitors of angiogenesis.

The concept that new blood vessels are needed for tumor growth and metastases was put forth by Folkman in the 1970s. Within the past seven years, Folkman's lab has discovered several peptides and proteins that could inhibit angiogenesis, among them angiostatin, endostatin, and small peptide derivatives of collagen and other basement membrane proteins.

These therapeutic agents offer a new way to attack cancer by attacking the vessels that feed the tumor cells rather than attacking the tumor cells directly.

In conventional antitumor therapy, chemotherapy targets the high growth rate of the cancer cells and is often tailored to target specific oncogenes or receptors expressed by a subtype of cancer or an organ-specific cancer. Because tumor cells are inherently genetically unstable, they often circumvent chemotherapeutic agents, either by changing their genetic makeup or by becoming drug resistant. Similarly, agents that are effective against one particular type of cancer fail against a cancer in a different/organ site.

A second big disadvantage to standard chemotherapy is the severe toxicity to normal cells that have a high rate of division—cells such as blood and bone marrow cells, gastrointestinal cells, and cells of the hair follicles.

Thus, the biggest issues facing chemotherapy today are lack of specificity (resulting in toxic side effects) and drug resistance because of high tumor-cell mutation rates.

Targeting endothelial cells may circumvent these problems and may also offer a means to combat metastatic spread. Because endothelial cells do not normally proliferate, they have not evolved the adaptive ability to mutate rapidly and are less likely to develop drug resistance.

While considerable debate exists over whether endothelial cells at different organ sites are the same, it is certain that they respond similarly to biochemical signals and stimuli that trigger their migration and proliferation.

Thus, researchers and clinicians can achieve a more universal approach to cancer treatment by targeting the endothelial cells rather than the tumor cells, with the potential of circumventing the nonspecific toxicity often associated with chemotherapy.

Antiangiogenic drugs affecting the genetically stable endothelial cells are also less likely to engender drug resistance.

Lastly, anti-angiogenesis therapy is designed to “starve” the tumor and eliminate the vasculature necessary for metastatic spread. Specifically, unlike existing cancer therapies, which target the tumor, agents that inhibit tumor angiogenesis-like endostatin—would target the tumor's life-support system. Effective treatment with angiogenesis inhibitors should result in tumors too “starved” to grow larger and should prevent existing micrometastases that have broken off from the primary tumor from developing the vasculature to grow into clinically significant tumors. In addition, these agents could cause regression of advanced primary tumors and metastases. Because they would be highly specific to tumor blood vessels, their use could avoid damage to normal cells and the associated side effects.

Angiogenesis likely plays different roles in the various types of cancer.

Patients most likely to benefit from anti-angiogenic therapy are those with early-stage, localized disease; ideally, physicians would reduce the tumor burden in patients with locally advanced disease enough to attempt curative surgery or to apply aggressive chemotherapy. In addition, patients known to be genetically susceptible to cancer could take angiogenesis inhibitors as preventive measures.

The vast majority of cancers are diagnosed late in their natural history. Consequently, in most patients, oncologists must control the disease not only at its site of origin (the primary tumor) but also at distant sites (metastases). Surgery, radiation therapy, and chemotherapy are the major tools available to accomplish these goals, but the high mortality associated with many cancers underscores the inadequacies of these treatments.

In each case, these treatments fail because of the following reasons:
The toxicity of the treatment outweighs the effect that the therapy has on the disease.
All cancer cells are not eradicated by the treatment because malignant cells develop resistance to radiation or chemotherapy or are too widely disseminated to be treated by radiation or surgery.

Cytotoxic chemotherapy often requires the oncologist to balance the treatment's efficacy with its morbidity. Although pharmaceuticals derive their power from their systemic effects, cytotoxic chemotherapeutics—most of which single out actively proliferating cells—also destroy normal cells that divide rapidly.

The destruction of both normal and cancer cells produces several unwelcome side effects:
Anemia and neutropenia (loss of immune cells [thrombocytopenia]), from destruction of bone marrow.
Nausea and vomiting, from damage to the gastrointestinal lining.
Death of hair follicles.
Damage to the nervous system.

The current therapies—surgery, radiation, and cytotoxic chemotherapy—have improved cancer treatment as much as is possible using these modalities. It seems certain that achieving further improvements will require exploitation of knowledge of cancer's molecular pathogenesis.

J. Folkman's 1971 New England Journal of Medicine paper (volume 285, page 1182-1186) introduced the idea that angiogenesis was critical to the pathogenesis of cancer and suggested that normal tissues that interact with the tumor might be targets for anticancer therapy.

Preclinical efficacy studies in the primary Lewis lung carcinoma and metastatic B16 xenograft studies demonstrated tumour stasis following subcutaneous administration of endostatin. Immunohisto-chemistry demonstrated that this effect was mediated through the inhibition of tumour angiogenesis. When endostatin therapy treatment was continued, the tumours remained in a dormant state, and importantly no evidence of drug resistance or toxic effects were seen. Indeed, this lack of toxicity was again shown in formal toxicology studies, thus demonstrating an immediate advantage over many of the anti-angiogenic compounds in development at present.

Data produced by other research groups, including the NCI had failed to show any anti-tumour effects. However, the reasons for the conflicting results were identified and the NCI now appears to be satisfied with the anti-angiogenic properties of endostatin.

In July 1999, the FDA approved the Investigational New Drug (IND) application for endostatin for the treatment of patients with solid tumours, enabling the NCI and EntreMed to initiate the three planned Phase I clinical studies with the recombinant protein.

The first study was initiated in September 1999 at the Dana-Farber Cancer Institute in Boston in patients with various solid tumours. The NCI sponsored the other Phase I clinical studies which were conducted at the Anderson Cancer Centre in Houston and the University of Wisconsin. Patients received daily intravenous doses of endostatin for 28 day cycles, with the patients at the Boston and Wisconsin centres remaining on the same daily dose of drug whilst the patients at Houston received increasing doses at eight week intervals if the disease was stable.

Preliminary results reported from the studies, in the 61 patients administered endostatin, showed that twelve patients received between four and twelve months of endostatin therapy. Five of the twelve patients had stable disease for a minimum of four months, with two of these patients receiving therapy for more than 12 months.

In the trial conducted at the Anderson Centre, PET scanning showed a significant reduction in the tumour blood flow within patients administered endostatin. This observation was corroborated by the University of Wisconsin study which showed that after 56 days of endostatin treatment, whilst the blood flow through the heart was unchanged, the blood flow in the tumours of some of the patients was reduced. A dose-related reduction in urine basic fibroblast growth factor (bFGF) and vascular endothelial growth factor (VEGF) levels was also observed.

Importantly, no major toxic side effects were reported in any of the studies and drug resistance did not appear to be a problem.

The combined data from all three Phase I clinical trials showed that although endostatin was well tolerated, only two out of the nineteen patients enrolled continued to receive the therapy whilst twelve patients were taken off the study due to disease progression, and a further five patients voluntarily withdrew from the study.

EntreMed has one ongoing Phase I clinical trial in Europe that is assessing the continuous infusion and subcutaneous administration of endostatin.

EntreMed plans to initiate a Phase II clinical study in humans with the end-point likely to be time to tumour progression as opposed to tumour shrinkage.

As with the vast majority of anti-angiogenic agents in development, the greatest potential for their use is likely to be in combination with chemo or radiotherapy. Indeed, some preclinical studies have shown that endostatin has synergistic effects with radiotherapy and EntreMed has several ongoing preclinical studies investigating various combination therapies. Preclinical studies assessing the efficacy of endostatin in models of progressive macular degeneration and rheumatoid arthritis (RA) are also ongoing.

Angiostatin treatment has also been shown to correlate with a decreased expression of the mRNA for both VEGF and bFGF. The human recombinant version successfully inhibited lung melanoma in the B 16 melanoma metastasis model. Three days after the injection of the tumour cells, animals were treated for 11 days with angiostatin. This treatment reduced lung metastases by 60-80%.

EntreMed completed preclinical toxicology and pharmacology results and submitted an IND application in December 1999. This application was accepted by the FDA in February 2000, and the first Phase I clinical study investigating angiostatin as a monotherapy was initiated in March 2000 at the Thomas Jefferson University Hospital in Philadelphia.

EntreMed initiated a second trial in July 2000 at the same hospital, but unlike endostatin, this study is investigating the product as part of a combination with radiotherapy in patients with advanced cancer. In both of these studies, angiostatin is being intravenously administered.

A European study for angiostatin began in November 2000 and is looking at the tolerability of Angiostatin when administered subcutaneously.

### SUMMARY OF THE INVENTION

The invention relates to proteins comprising anti-angiogenic peptides or fragments or variants thereof fused to albumin or fragments or variants thereof. These fusion proteins are herein collectively referred to as “albumin fusion proteins of the invention.” These fusion proteins of the invention exhibit extended in vivo half-life and/or extended or therapeutic activity.

The invention encompasses therapeutic albumin fusion proteins, compositions, pharmaceutical compositions, formulations and kits. The invention also encompasses nucleic acid molecules encoding the albumin fusion proteins of the invention, as well as vectors containing these nucleic acids, host cells transformed with these nucleic acids and vectors, and methods of making the albumin fusion proteins of the invention using these nucleic acids, vectors, and/or host cells.

The invention also relates to compositions and methods for inhibiting proliferation and/or migration of endothelial cells; inhibiting tumor-induced angiogenesis; inhibiting growth of or promoting regression of, primary tumors and metastases; and for treating cancer, diabetic retinopathy, progressive macular degeneration or rheumatoid arthritis and all angiogenesis related diseases.

The invention also relates to methods of targeting an antiangiogenic peptide to the inside of a cell or at cell structures in a mammal; methods of targeting the albumin fusion proteins of the invention to a cell type, target organ, or a specific cytological or anatomical location; methods of diagnosing an anti-angiogenesis related disease or disorder in a mammal; and methods of improving the scheduling of dosing of an antiangiogenic peptide.

### BRIEF DESCRIPTION OF THE DRAWINGS

FIG. 1. DNA sequence of the N-terminal endostatin-albumin fusion open reading frame. (This DNA sequence encodes the primary translation product and, therefore, the first 72 nucleotides encode a 24 amino acid leader sequence which is removed during secretion from yeast in the examples herein).

FIG. 2. Amino acid sequence of the N-terminal endostatin-albumin fusion protein. (This amino acid sequence represents the primary translation product of the DNA sequence shown in FIG. 1 and, therefore, includes a 24 amino acid leader sequence which is removed during secretion in yeast. Thus, the protein sequence does not represent the sequence of the protein used in the tumor inhibition examples herein).

FIG. 3. DNA sequence of the C-terminal albumin-endostatin fusion open reading frame. (This DNA sequence encodes the primary translation product and, therefore, the first 72 nucleotides encode a 24 amino acid leader sequence which is removed during secretion from yeast in the examples herein).

FIG. 4. Amino acid sequence of the C-terminal albumin-endostatin fusion protein. (This amino acid sequence represents the primary translation product of the DNA sequence shown in FIG. 3 and, therefore, includes a 24 amino acid leader sequence which is removed during secretion in yeast. Thus, the protein sequence does not represent the sequence of the protein used in the tumor inhibition examples herein).

FIG. 5. DNA sequence of the N-terminal angiostatin(non-glycosylated)-albumin fusion open reading frame.

FIG. 6. Amino acid sequence of the N-terminal angiostatin(non-glycosylated)-albumin fusion protein.

FIG. 7. DNA sequence of the C-terminal albumin-angiostatin(non-glycosylated)-fusion open reading frame.

FIG. 8. Amino acid sequence of the C-terminal albumin-angiostatin(non-glycosylated)-fusion protein.

FIG. 9. DNA sequence of the N-terminal Kringle5-(GGS)4GG-albumin fusion open reading frame.

FIG. 10. Amino acid sequence of the N-terminal Kringle5-(GGS)4GG-albumin fusion protein.

FIG. 11. DNA sequence of the C-terminal albumin-(GGS)4GG-Kringle5 fusion open reading frame.

FIG. 12. Amino acid sequence of the C-terminal albumin-(GGS)4GG-Kringle 5 fusion protein.

FIG. 13. 4-12% Gradient SDS Gel and Western Blot: A. Colloidal Blue Gel. B. Anti-endostatin Western Blot. C. Anti-HSA Western Blot.

FIG. 14. FIG. 14. Mean endostatin concentrations+/−SD, following s.c. application.

FIG. 15. FIG. 15. Mean endostatin concentrations+/−SD, following i.v. application.

FIG. 16. PK Data. Treatment=C terminal-endostatin 72 h, route=s.c., loading dose=1.8, maintenance dose=1.2

FIG. 17. PK Data. Treatment=C terminal-endostatin 24 h, route=s.c., loading dose=1.5, maintenance dose=0.5

FIG. 18. PK Data. Treatment=N terminal-endostatin 72 h, route=s.c., loading dose=1, maintenance dose=0.9

FIG. 19. PK Data. Treatment=N terminal-endostatin 24 h route=s.c., loading dose=0.8, maintenance dose=0.25

FIG. 20. Efficacy of albumin-fused-endostatin and classic endostatin in a migration-assay (HUVEC). All concentrations or dosages for the fusions are related to endostatin equivalents.

FIG. 21. Tumor volume after treatment of Bx Pc-3 with albumin-fused-C-terminal-endostatin s.c. Control=o----o; 1.2 mg/kg/72 h=□----□; 0.5 mg/kg/24 hr=∘----∘. All concentrations or dosages for the fusions are related to endostatin equivalents.

FIG. 22. Tumor Volume after treatment of Bx Pc-3 with albumin-fused-C terminal-endostatin s.c. Control=o----o; 0.4 mg/kg/72 h=⋄----⋄; 1.2 mg/kg/72 h=□----□; 3.6 mg/kg/72 h=□-----□. All concentrations or dosages for the fusions are related to endostatin equivalents.

FIG. 23. Tumor Volume after treatment of Bx Pc-3 with albumin-fused-N terminal-endostatin s.c. Control=□----□; 0.8 mg/kg/72 h=□----□; 0.75 mg/kg/48 hr=o----o; 0.4 mg/kg/24 h=∘----∘. All concentrations or dosages for the fusions are related to endostatin equivalents.

FIG. 24. Tumor Volume after treatment of Bx Pc-3 with albumin-fused-N terminal-endostatin s.c. Control=o----o; 0.25 mg/kg/48 h=Δ----Δ; 0.75 mg/kg/48 h=□----□; 2.25 mg/kg/48 h=Δ----Δ. All concentrations or dosages for the fusions are related to endostatin equivalents.

FIG. 25. SDS PAGE of C-terminal rHA Angiostatin purified on SP-FF.

FIG. 26. Western Blot analysis of C-terminal rHA Angiostatin.

FIG. 27. SDS PAGE of yeast cell supernatants expressing albumin or antiostatin-albumin fusion proteins.

FIG. 28 (A-D). Amino acid sequence of a mature form of human albumin (SEQ ID NO:18) and a polynucleotide encoding it (SEQ ID NO:17).

### DETAILED DESCRIPTION OF THE INVENTION

The present invention relates to fusion proteins comprising albumin coupled to angiogenesis inhibiting peptides. The terms “protein” and “peptide” as used herein are not limiting and include proteins, polypeptides as well as peptides. These peptides include, but are in no way limited to, endostatin (including restin, arresten, canstatin and tumstatin) or fragments or variants thereof, which have angiogenesis inhibiting properties; angiostatin or fragments or variants thereof, which have angiogenesis inhibiting properties; alphastatin or fragments or variants thereof, which have angiogenesis inhibiting properties; kringle 5 or fragments or variants thereof, which have angiogenesis inhibiting properties; and anti-thrombin III or fragments or variants thereof, which have angiogenesis inhibiting properties.

The present invention also relates to bifunctional (or multifunctional) fusion proteins in which albumin is coupled to two (or more) angiogenesis inhibiting peptides, optionally different angiogenesis inhibiting peptides, including but not limited to endostatin/angiostatin or endostatin/angiostatin/kringle 5, fusions, or fragments or variants thereof, which have angiogenesis inhibiting properties. Such bifunctional (or multifunctional) fusion proteins may also exhibit synergistic anti-angiogenic effects, as compared to an albumin fusion protein comprising only one type of angiogenesis inhibiting peptide.

The present invention also relates to fusion proteins in which one (or more) angiogenesis inhibiting peptide(s), optionally different angiogenesis inhibiting peptides, is coupled to two albumin molecules or fragments or variants of albumin, which could be the same or different.

Furthermore, chemical entities may be covalently attached to the fusion proteins of the invention or used in combinations to enhance a biological activity or to modulate a biological activity.

The albumin fusion proteins of the present invention are expected to prolong the half-life of the angiogenesis inhibiting peptide in vivo. The in vitro or in vivo half-life of said albumin-fused peptide is extended 2-fold, 5-fold, or more, over the half-life of the peptide lacking the linked albumin. Furthermore, due at least in part to the increased half-life of the peptide, the albumin fusion proteins of the present invention are expected to reduce the frequency of the dosing schedule of the therapeutic peptide. The dosing schedule frequency is reduced by at least one-quarter, or by at least one-half or more, as compared to the frequency of the dosing schedule of the therapeutic peptide lacking the linked albumin.

The albumin fusion proteins of the present invention prolong the shelf-life of the peptide, and/or stabilize the peptide and/or its activity in solution (or in a pharmaceutical composition) in vitro and/or in vivo. These albumin-fusion proteins, which may be therapeutic agents, are expected to reduce the need to formulate protein solutions with large excesses of carrier proteins (such as albumin, unfused) to prevent loss of proteins due to factors such as nonspecific binding.

The present invention also encompasses nucleic acid molecules encoding the albumin fusion proteins as well as vectors containing these nucleic acids, host cells transformed with these nucleic acids vectors, and methods of making the albumin fusion proteins of the invention using these nucleic acids, vectors, and/or host cells. The present invention further includes transgenic organisms modified to contain the nucleic acid molecules of the invention, optionally modified to express the albumin fusion proteins encoded by the nucleic acid molecules.

The present invention also encompasses pharmaceutical formulations comprising an albumin fusion protein of the invention and a pharmaceutically acceptable diluent or carrier. Such formulations may be in a kit or container. Such kit or container may be packaged with instructions pertaining to the extended shelf-life of the protein. Such formulations may be used in methods of preventing, treating or ameliorating an angiogenesis-related disease, disease symptom or a related disorder in a patient, such as a mammal, or a human, comprising the step of administering the pharmaceutical formulation to the patient.

The invention also encompasses a method for potentially minimizing side effects (e.g., injection site reaction, headache, nausea, fever, increased energy levels, rash asthenia, diarrhea, dizziness, allergic reactions, abnormally low neutrophil levels) associated with the treatment of a mammal with angiogenesis inhibiting peptide in moderately higher concentrations comprising administering an albumin-fused angiogenesis inhibiting peptide of the invention to said mammal.

The present invention encompasses a method of preventing, treating or ameliorating an angiogenesis-related disease or disorder caused by angiogenesis comprising administering to a mammal, in which such prevention treatment, or amelioration is desired an albumin fusion protein of the invention that comprises an angiogenesis inhibiting peptide (or fragment or variant thereof) in an amount effective to treat prevent or ameliorate the disease or disorder. In the present invention, the angiogenesis inhibiting peptide, such as endostatin, is also called the “Therapeutic protein”.

The present invention encompasses albumin fusion proteins comprising an endostatin peptide or multiple copies of monomers of endostatin (including fragments and variants thereof) fused to albumin or multiple copies of albumin (including fragments and variants thereof).

The present invention also encompasses a method for extending the half-life of endostatin peptide in a mammal. The method entails linking endostatin peptide to an albumin to form albumin-fused endostatin peptide and administering the albumin-fused endostatin peptide to a mammal. Typically, the half-life of the albumin-fused endostatin peptide may be extended by at least 2-fold, 5-fold, 10-fold, 20-fold, 30-fold, 40-fold or at least 50-fold over the half-life of the endostatin peptide lacking the linked albumin.

Exemplified herein are fusion proteins comprising albumin fused to endostatin which exhibit anti-tumor activity. Such anti-tumor activity includes, but is not limited to, the inhibition of growth of primary tumors or metastases. Further, the invention relates to the use of such fusion proteins comprising albumin fused to endostatin for treating cancer, diabetic retinoplasty, progressive macular degeneration or rheumatoid arthritis.

Various aspects of the present invention are discussed in further detail below.

Endostatin

Endostatin was first described in 1997, (M. O'Reilly, et al., Cell 88:277-285), as a 20 kDa C-terminal fragment of collagen XVIII, which was originally isolated from a haemangioendothelioma cell line in 1996. The original study describing the anti-angiogenic effects of endostatin used a recombinant murine version produced in baculovirus and E. coli expression systems. This molecule demonstrated selective inhibition of endothelial cell proliferation in vitro in the cell adhesion molecule (CAM) assay.

Collagen XVIII, a component of the basal lamina that surrounds Vascular Endothelial Cells (VECs), is the parent protein of endostatin, and zinc is known to be necessary for is anti-angiogenic activity. VECs must begin basal lamina degradation before they begin migrating toward an angiogenic source. In cell culture studies, endostatin's primary function seems to be inhibition of VEC proliferation, possibly by preventing Endothelian Cell Matrix (ECM) remodeling by the proteinase collagenase.

Endostatin has a molecular weight of approximately 18,000 to approximately 20,000 Daltons (18 to 20 kDa) and is capable of inhibiting endothelial cell proliferation in cultured endothelial cells. One version of the protein can be further characterized by the N-terminal amino acid sequence His Thr His Gln Asp Phe Gin Pro Val Leu His Leu Val Ala Leu Asn Thr Pro Leu Ser (SEQ ID NO: 1), as identified in U.S. Pat. No. 5,854,205 which corresponds to a C-terminal fragment of murine collagen type XVIII. The corresponding N-terminal amino acid sequence of a C-terminal fragment of human collagen type XVIII, which was used in the examples herein, is His Ser His Arg Asp Phe Gln Pro Val Leu His Leu Val Ala Leu Asn Ser Pro Leu Ser (SEQ ID NO: 2).

An endostatin peptide useful in the present invention includes fragments or variants of endostatin, such as any molecule which is an analog, homolog, fragment, or a derivative of naturally occurring endostatin peptide, such as those described in U.S. Pat. No. 5,854,205 which is specifically incorporated by reference herein. Active fragments and variants thereof which are useful in the albumin fusion proteins of the present invention can be identified using methods known in the art, including those described in the patents and references listed in Table 1, which are incorporated by reference herein. The endostatin peptide useful in the present invention need only possess a single biological activity of the endostatin peptide corresponding to SEQ ID NO:1 or SEQ ID NO:2.

The endostatin peptides useful in the invention exhibit anti-angiogenesis activity, and may, further, possess additional advantageous features, such as, for example, increased bioavailability, and/or stability, or reduced host immune recognition.

Active fragments and variants thereof which are useful in the albumin fusion proteins of the present invention can be identified using methods known in the art including those described in the patents and references listed in Table 1, which are incorporated by reference herein.

When endostatin (or a fragment or variant thereof) is to be expressed in yeast which is capable of O-glycosylation, any serines or threonines may be modified or otherwise decreased in number to minimize the effect of O-glycosylation or the biological activity of endostatin (or a fragment or variant thereof). Alternatively, or in addition, use of a yeast strain which underglycosylates (i.e., which is deficient in O-glycosylation) may be used.

Angiostatin

Angiostatin is a fragment of plasminogen, originally discovered in 1994, that was shown to have anti-angiogenic activity. Angiostatin binds ATP synthase on the surface of endothelial cells (ECs) and inhibits EC migration and tubule formation, as well as inducing apoptosis in both ECs and tumour cells.

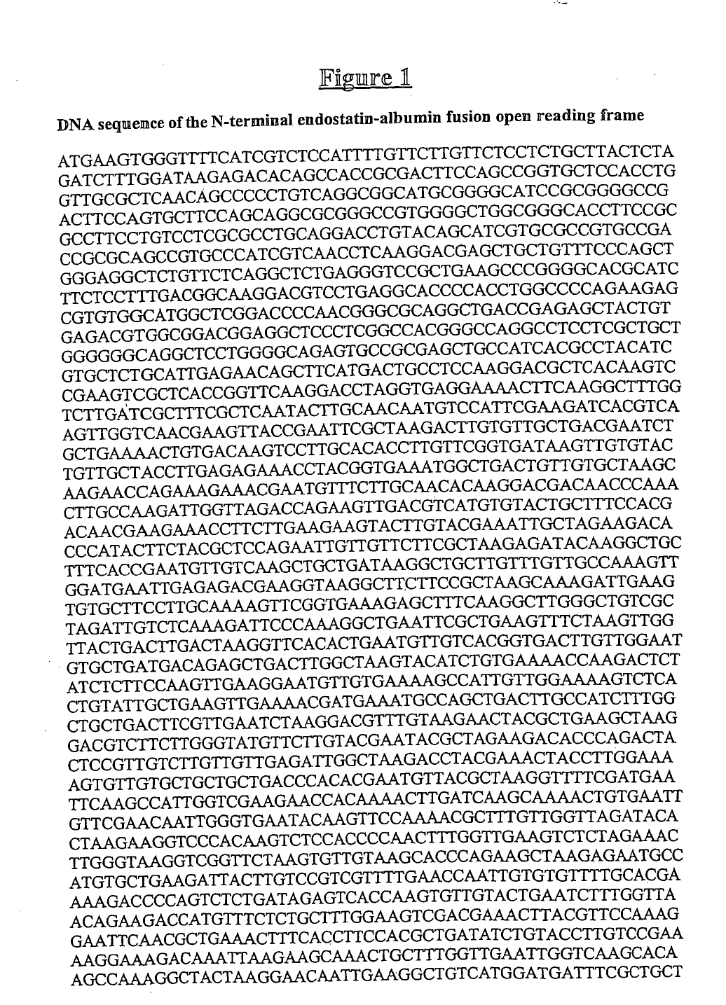

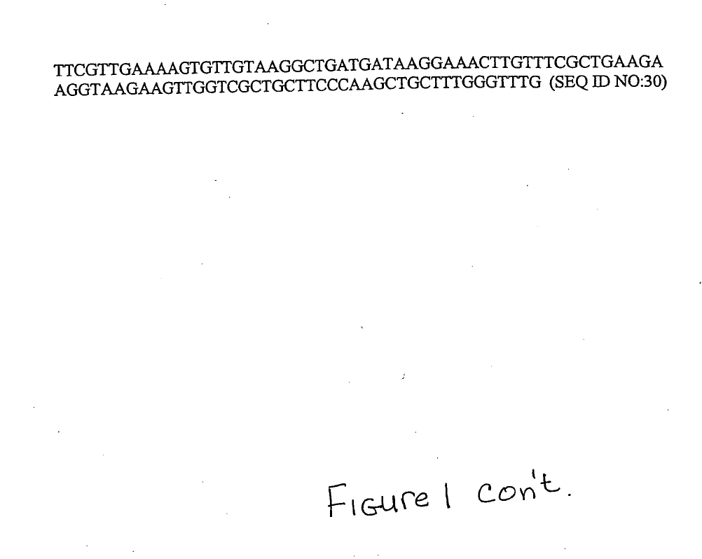

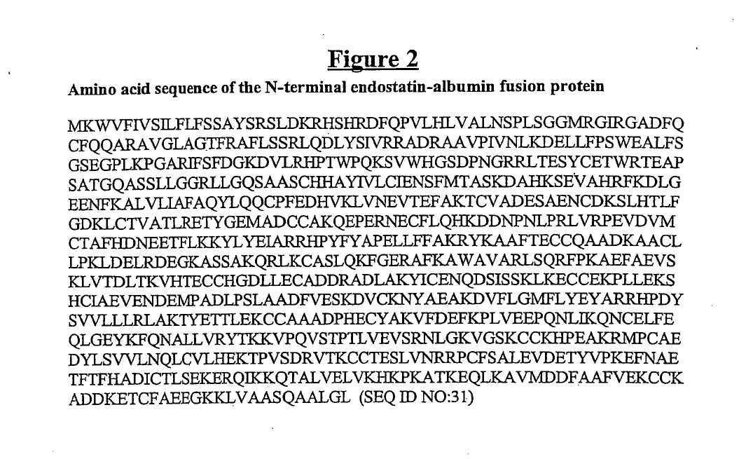

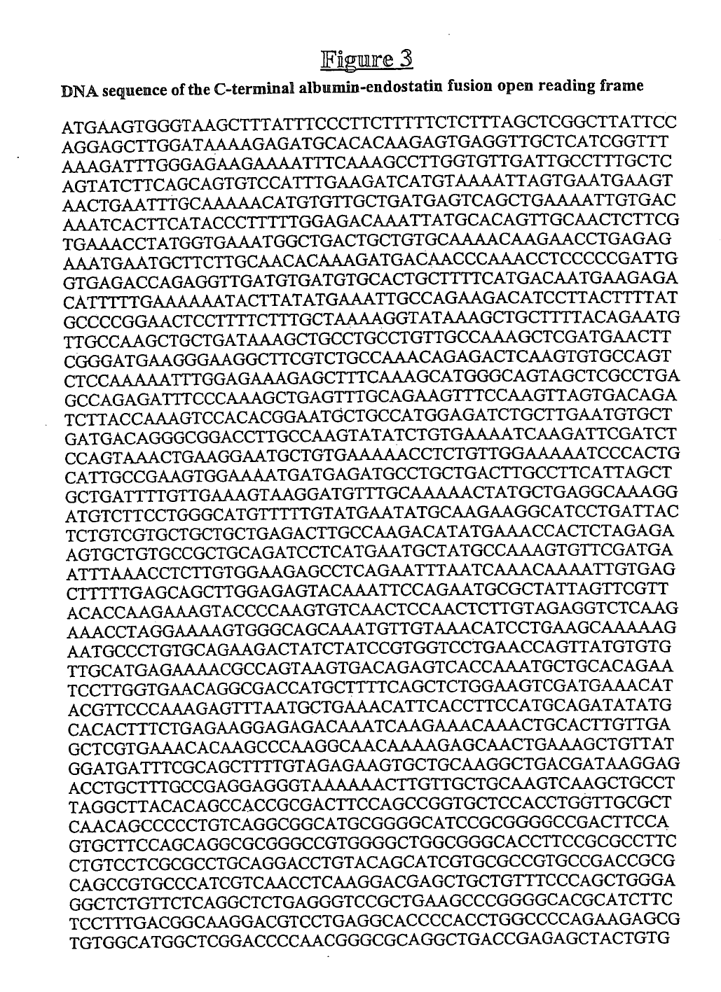

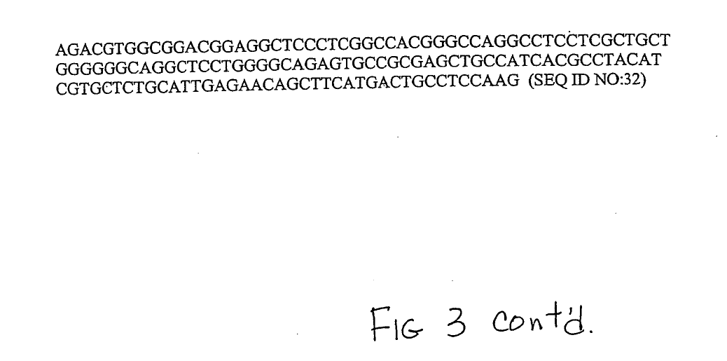

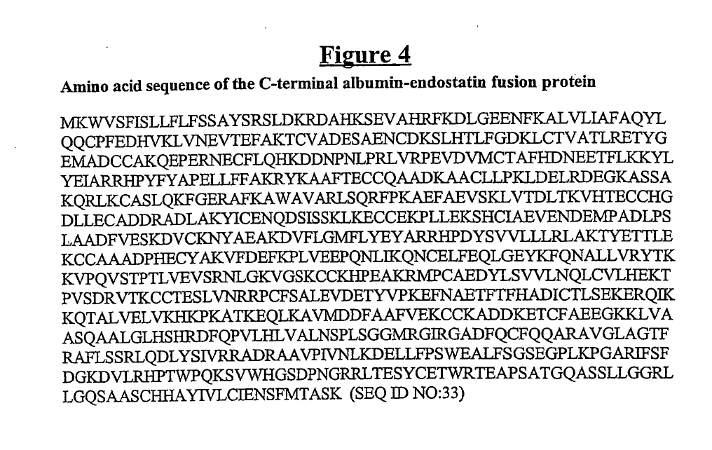

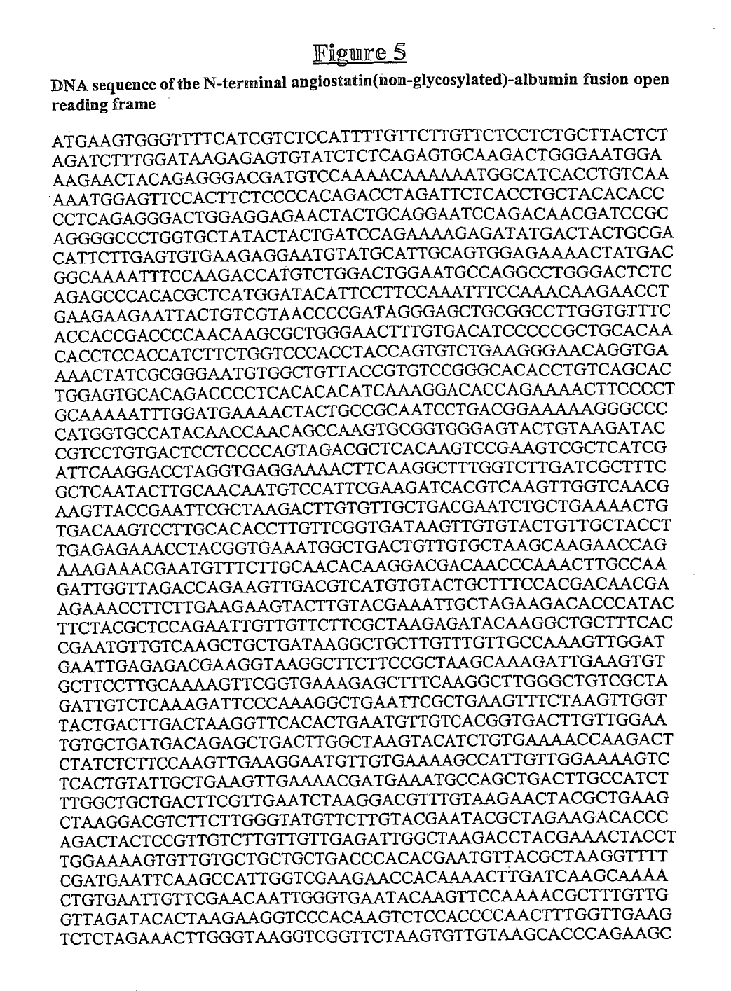

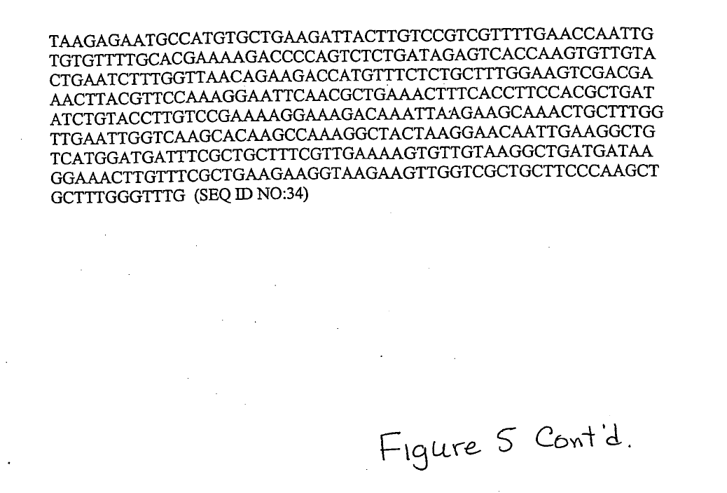

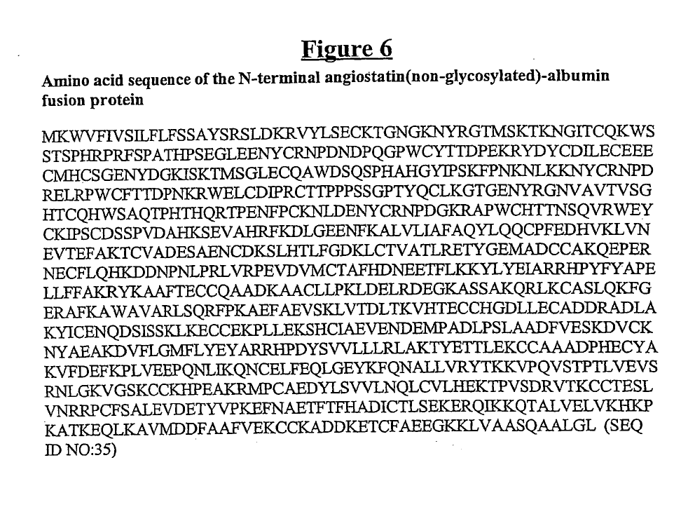

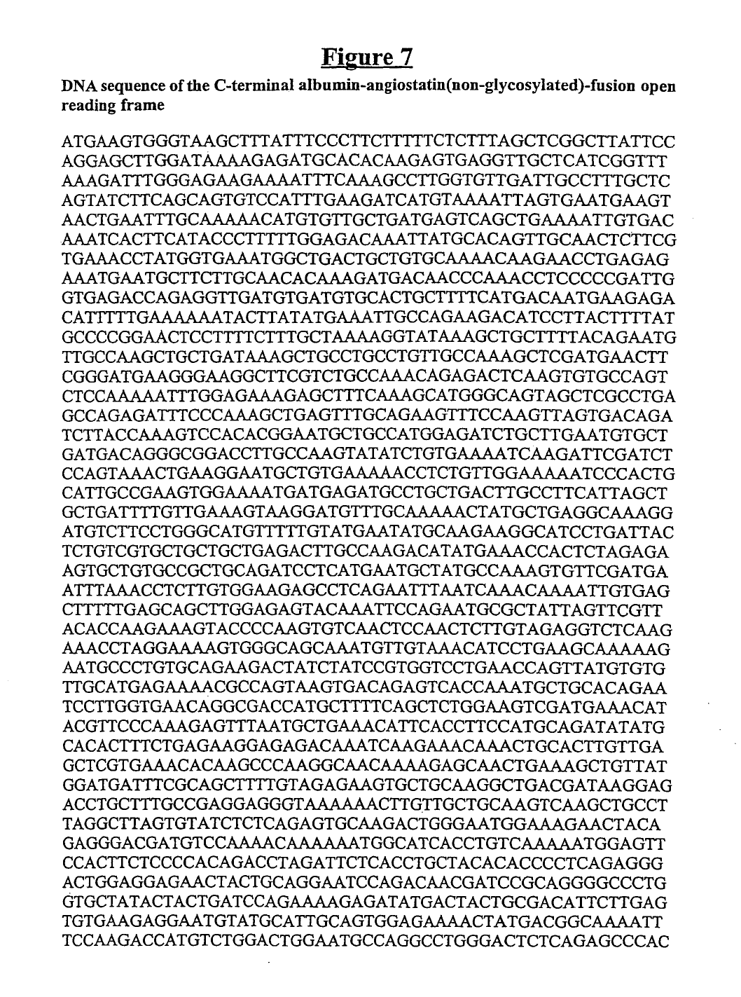

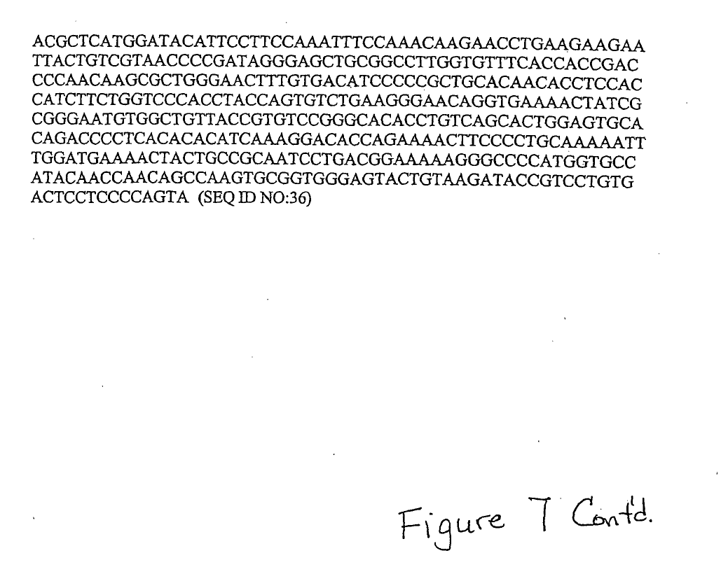

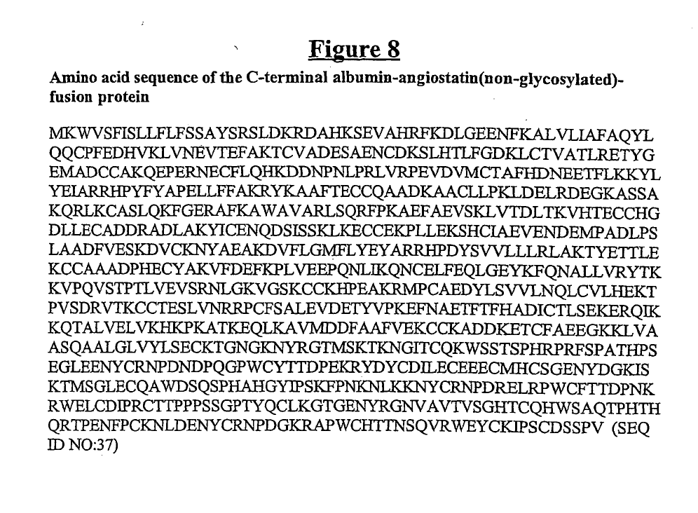

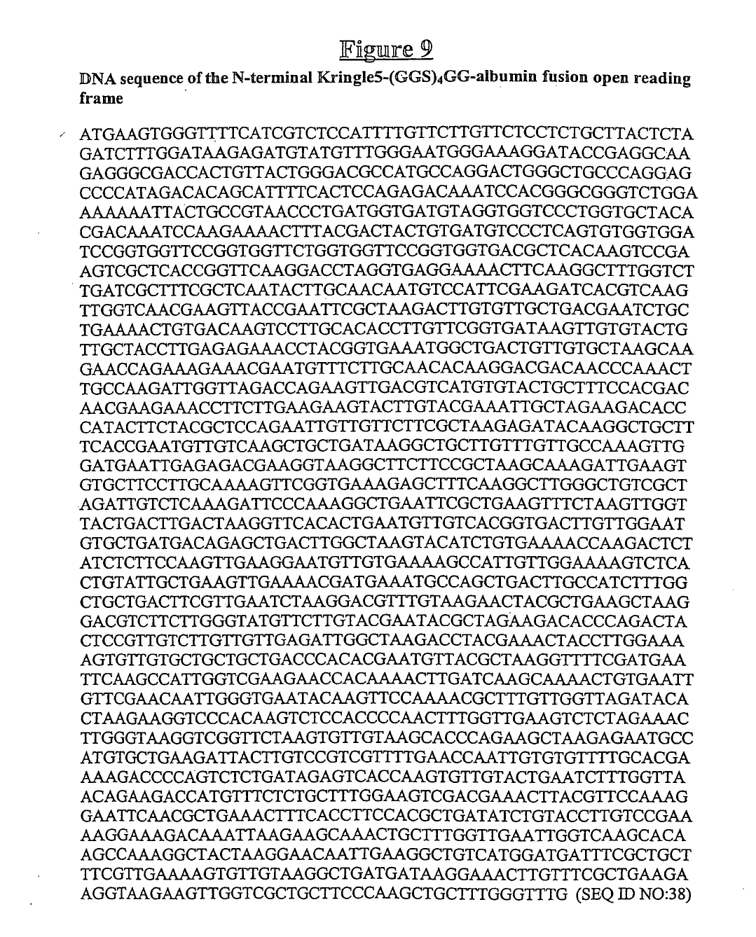

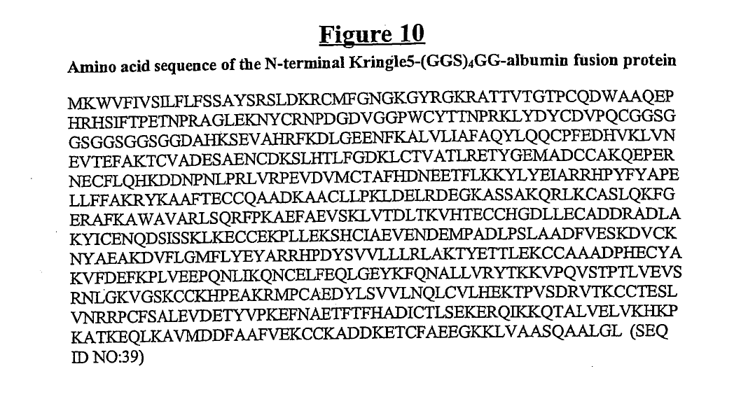

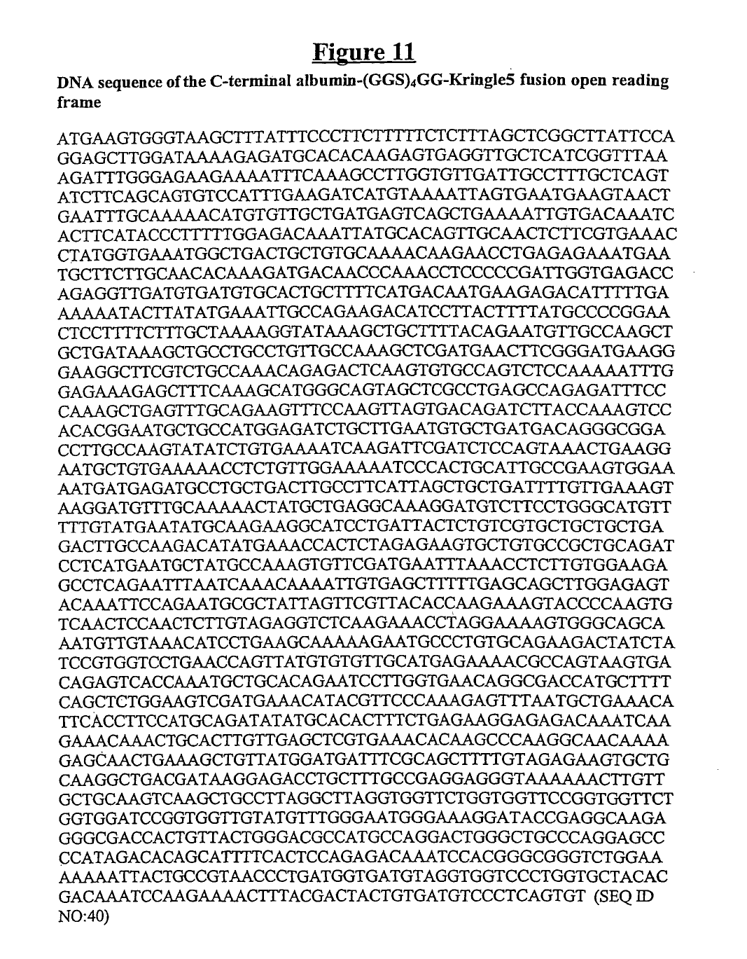

“Angiostatin” has been defined by its ability to overcome the angiogenic activity of endogenous growth factors such as bFGF, in vitro, and by it amino acid sequence homology and structural similarity to an internal portion of plasminogen beginning at approximately plasminogen amino acid 98 as shown in FIGS. 1A and 1B of U.S. Pat. No. 5,885,795. Angiostatin comprises a protein having a molecular weight of between approximately 38 kDa and 45 kDa as determined by reducing polyacrylamide gel electrophoresis and having an amino acid sequence substantially similar to that of a fragment of murine plasminogen beginning at amino acid number 98 of an intact murine plasminogen molecule.

The amino acid sequence of angiostatin varies slightly between species. For example, in human angiostatin the amino acid sequence is substantially similar to the sequence of the above described murine plasminogen fragment, although an active human angiostatin sequence may start at either amino acid number 97 or 99 of an intact human plasminogen amino acid sequence. Further, fragments of human plasminogen has similar anti-angiogenic activity as shown in a mouse tumor model. It is to be understood that the number of amino acids in the active angiostatin molecule may vary and all amino acid sequences that have endothelial inhibiting activity are contemplated as being included in the present invention. See, e.g., U.S. Pat. No. 5,885,795.

An “Angiostatin” peptide useful in the present invention includes fragments or variants thereof, such as any molecule which is an analog, homolog, fragment, or a derivative of naturally occurring angiostatin, such as those described in U.S. Pat. No. 5,885,795 which is specifically incorporated by reference herein.

Angiostatin has a specific three dimensional conformation that is defined by the kringle region of the plasminogen molecule (Robbins, K. C., “The plasminogen-plasmin enzyme system” Hemostasis and Thrombosis, Basic Principles and Practice, 2nd Edition, ed. by Colman, R. W. et al. J. B. Lippincott Company, pp. 340-357, 1987). There are five such kringle regions, which are conformationally related motifs and have substantial sequence homology, in the NH₂ terminal portion of the plasminogen molecule. The three dimensional conformation of angiostatin is believed to encompass plasminogen kringle regions 1 through 3 and a part of kringle region 4. Each kringle region of the plasminogen molecule contains approximately 80 amino acids and contains 3 disulfide bonds. This cysteine motif is known to exist in other biologically active proteins. These proteins include, but are not limited to, prothrombin, hepatocyte growth factor, scatter factor and macrophage stimulating protein. (Yoshimura, T, et al., “Cloning, sequencing, and expression of human macrophage stimulating protein (MSP, MST1) confirms MSP as a member of the family of kringle proteins and locates the MSP gene on Chromosome 3” J. Biol. Chem., Vol. 268, No. 21, pp. 15461-15468, 1993). It is contemplated that any isolated protein or peptide having a three dimensional kringle-like conformation or cysteine motif that has anti-angiogenic activity in vivo, is part of the present invention.

The angiostatin peptides useful in the invention exhibit anti-angiogenesis activity, and may, further, possess additional advantageous features, such as, for example, increased bioavailability, and/or stability, or reduced host immune recognition.

Active fragments and variants thereof which are useful in the albumin fusion proteins of the present invention can be identified using methods known in the art, including those described in the patents and references listed in Table 1, which are incorporated by reference herein.

Kringle 5

Kringle 5 is an internal fragment of plasminogen which is outside the angiostatin structure but present in plasminogen. Kringle 5 displays about 50% sequence identity and structural similarity to the first four kringle domains of plasminogen. (Cao, Y et al, “Kringle domains of human Angiostatin” J. Biol. Chem. Vol. 271, No 46, pp 29461-99467, 1996; Cao, Y et al, “Kringle 5 of Plasminogen is a novel Inhibitor of Endothelial Cell Growth” J. Biol. Chem. Vol. 272, No 36, pp 22924-22928, 1997 and Lu, H, et al; “Kringle 5 causes cell cycle arrest and apoptosis of endothelial cells” Biochem. Biophysical Research Cummunications, Vol. 258, pp 668-673, 1999)

The Kringle 5 peptides useful in the invention exhibit anti-angiogenesis activity, and may, further, possess additional advantageous features, such as, for example, increased bioavailability, and/or stability, or reduced host immune recognition.

Active fragments and variants thereof which are useful in the albumin fusion proteins of the present invention can be identified using methods known in the art, including those described in the patents and references listed in Table 1, which are incorporated by reference herein.

Albumin

The terms, human serum albumin (HSA) and human albumin (HA) are used interchangeably herein. The terms, “albumin and “serum albumin” are broader, and encompass human serum albumin (and fragments and variants thereof) as well as albumin from other species (and fragments and variants thereof).

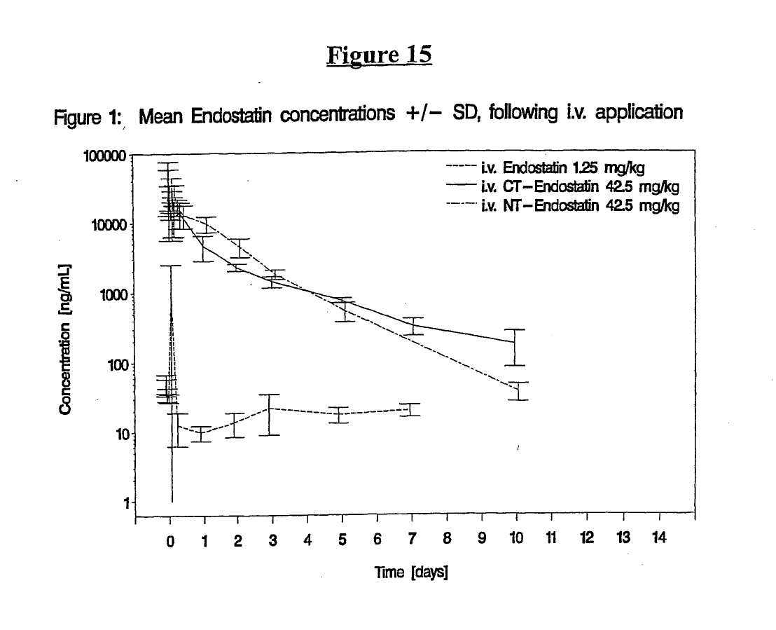

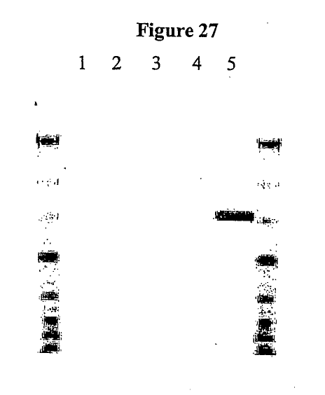

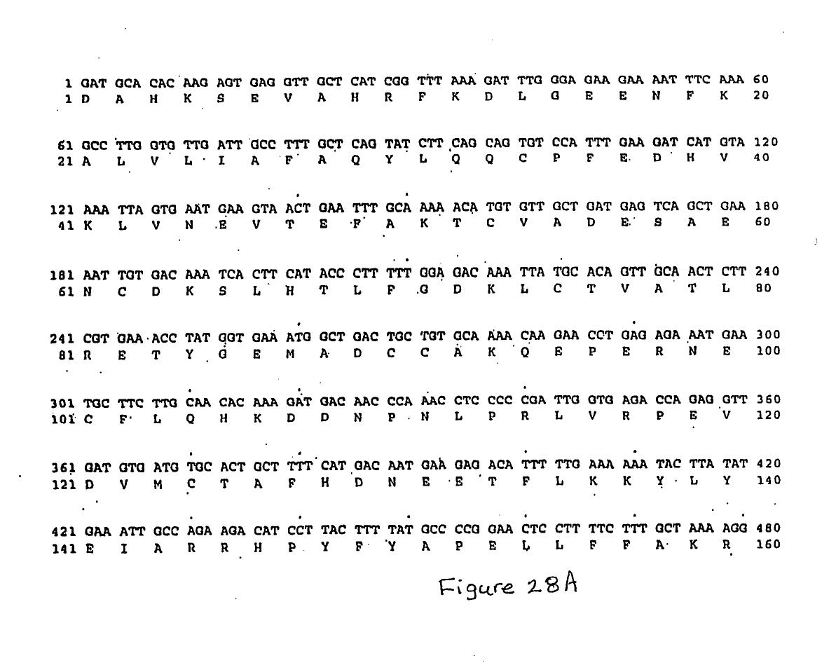

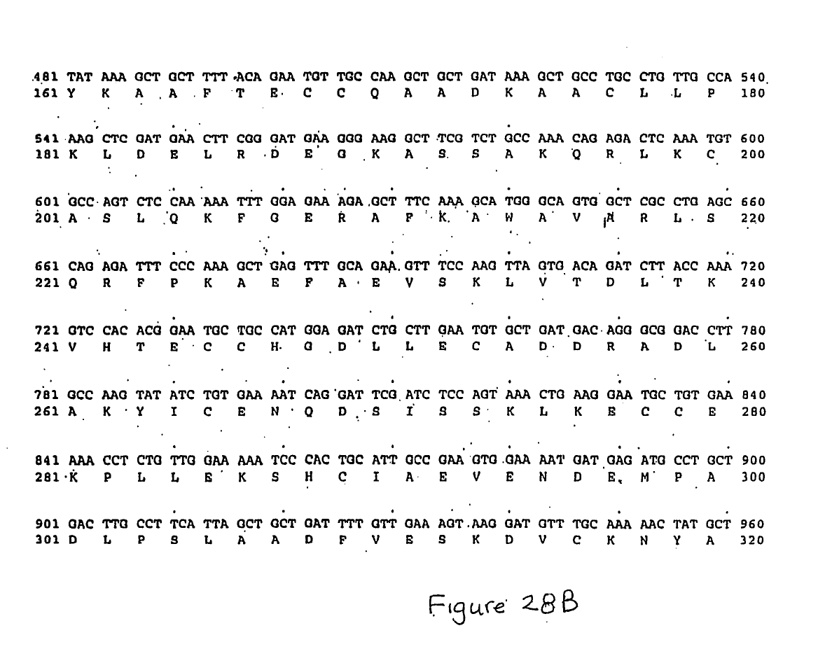

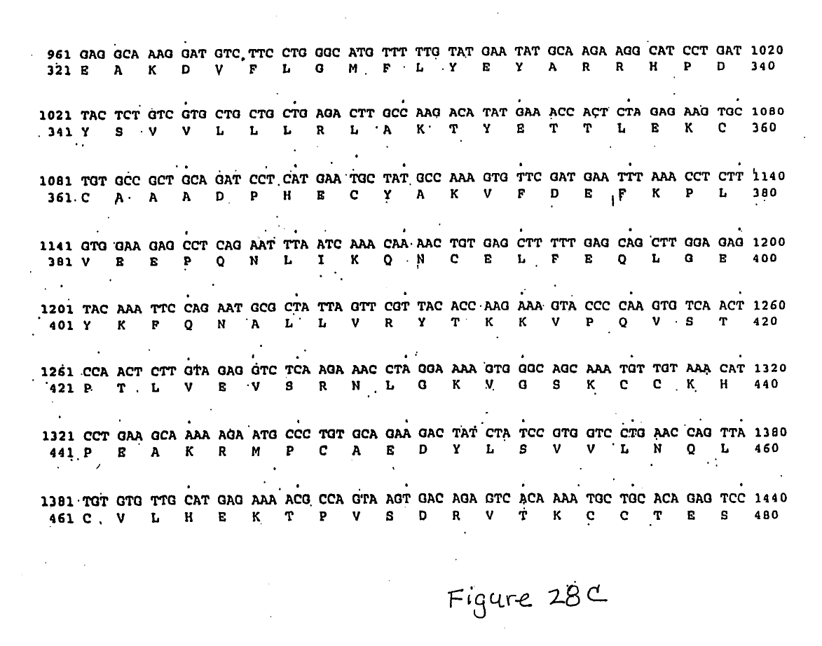

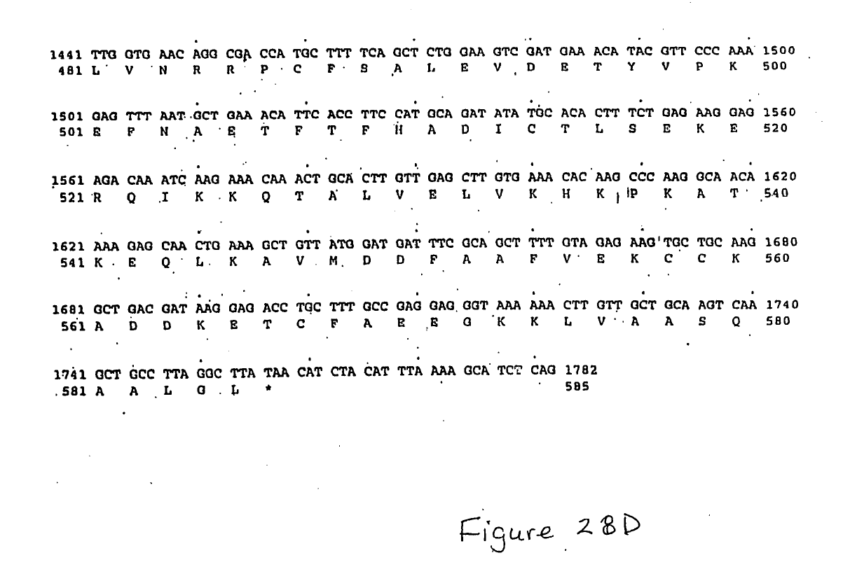

As used herein, “albumin” refers collectively to albumin protein or amino acid sequence, or an albumin fragment or variant, having one or more functional activities (e.g., biological activities) of albumin. In particular, “albumin” refers to human albumin or fragments thereof (see EP 201 239, EP 322 094 WO 97/24445, WO95/23857) especially the mature form of human albumin as shown in FIG. 27 and SEQ ID NO:18 herein and in FIG. 15 and SEQ ID NO:18 of U.S. Provisional Application Ser. No. 60/355,547 and WO 01/79480 or albumin from other vertebrates or fragments thereof, or analogs or variants of these molecules or fragments thereof.

The human serum albumin protein used in the albumin fusion proteins of the invention contains one or both of the following sets of point mutations with reference to SEQ ID NO:18: Leu-407 to Ala, Leu-408 to Val, Val-409 to Ala, and Arg-410 to Ala; or Arg-410 to Ala, Lys-413 to Gln, and Lys-414 to Gln (see, e.g., WO 95/23857, hereby incorporated in its entirety by reference herein). In other embodiments, albumin fusion proteins of the invention that contain one or both of above-described sets of point mutations have improved stability/resistance to yeast Yap3p proteolytic cleavage, allowing increased production of recombinant albumin fusion proteins expressed in yeast host cells.

As used herein, a portion of albumin sufficient to prolong or extend the in vivo half-life, therapeutic activity, or shelf-life of the Therapeutic protein refers to a portion of albumin sufficient in length or structure to stabilize, prolong or extend the in vivo half-life, therapeutic activity or shelf-life of the Therapeutic protein portion of the albumin fusion protein compared to the in vivo half-life, therapeutic activity, or shelf-life of the Therapeutic protein in the non-fusion state. The albumin portion of the albumin fusion proteins may comprise the full length of the HA sequence as described above, or may include one or more fragments thereof that are capable of stabilizing or prolonging the therapeutic activity. Such fragments may be of 10 or more amino acids in length or may include about 15, 20, 25, 30, 50, or more contiguous amino acids from the HA sequence or may include part or all of specific domains of HA.

The albumin portion of the albumin fusion proteins of the invention may be a variant of normal HA. The Therapeutic protein portion of the albumin fusion proteins of the invention may also be variants of the Therapeutic proteins as described herein. The term “variants” includes insertions, deletions and substitutions, either conservative or non conservative, where such changes do not substantially alter one or more of the oncotic, useful ligand-binding and non-immunogenic properties of albumin, or the active site, or active domain which confers the therapeutic activities of the Therapeutic proteins.

In particular, the albumin fusion proteins of the invention may include naturally occurring polymorphic variants of human albumin and fragments of human albumin, for example those fragments disclosed in EP 322 094 (namely HA (Pn), where n is 369 to 419). The albumin may be derived from any vertebrate, especially any mammal, for example human, cow, sheep, or pig. Non-mammalian albumins include, but are not limited to, hen and salmon. The albumin portion of the albumin fusion protein may be from a different animal than the Therapeutic protein portion.

Generally speaking, an HA fragment or variant will be at least 100 amino acids long, optionally at least 150 amino acids long. The HA variant may consist of or alternatively comprise at least one whole domain of HA, for example domains 1 (amino acids 1-194 of SEQ ID NO:18), 2 (amino acids 195-387 of SEQ ID NO:18), 3 (amino acids 388-585), 1+2 (1-387 of SEQ ID NO:18), 2+3 (195-585 of SEQ ID NO:18) or 1+3 (amino acids 1-194 of SEQ ID NO:18+amino acids 388-585 of SEQ ID NO:18). Each domain is itself made up of two homologous subdomains namely 1-105, 120-194, 195-291, 316-387, 388-491 and 512-585, with flexible inter-subdomain linker regions comprising residues Lys106 to Glu119, Glu292 to Va1315 and Glu492 to Ala511.

The albumin portion of an albumin fusion protein of the invention may comprise at least one subdomain or domain of HA or conservative modifications thereof. If the fusion is based on subdomains, some or all of the adjacent linker is may optionally be used to link to the Therapeutic protein moiety.

Albumin Fusion Proteins

The present invention relates generally to albumin fusion proteins and methods of treating, preventing, or ameliorating diseases or disorders. As used herein, “albumin fusion protein” refers to a protein formed by the fusion of at least one molecule of albumin (or a fragment or variant thereof) to at least one molecule of a Therapeutic protein (or fragment or variant thereof). An albumin fusion protein of the invention comprises at least a fragment or variant of a Therapeutic protein and at least a fragment or variant of human serum albumin, which are associated with one another, such as by genetic fusion (i.e., the albumin fusion protein is generated by translation of a nucleic acid in which a polynucleotide encoding all or a portion of a Therapeutic protein is joined in-frame with a polynucleotide encoding all or a portion of albumin) to one another. The Therapeutic protein and albumin protein, once part of the albumin fusion protein, may be referred to as a “portion”, “region” or “moiety” of the albumin fusion protein.

In one embodiment, the invention provides an albumin fusion protein comprising, or alternatively consisting of, a Therapeutic protein and a serum albumin protein. In other embodiments, the invention provides an albumin fusion protein comprising, or alternatively consisting of, a biologically active and/or therapeutically active fragment of a Therapeutic protein and a serum albumin protein. In other embodiments, the invention provides an albumin fusion protein comprising, or alternatively consisting of, a biologically active and/or therapeutically active variant of a Therapeutic protein and a serum albumin protein. In further embodiments, the serum albumin protein component of the albumin fusion protein is the mature portion of serum albumin.

In further embodiments, the invention provides an albumin fusion protein comprising, or alternatively consisting of, a Therapeutic protein, and a biologically active and/or therapeutically active fragment of serum albumin. In further embodiments, the invention provides an albumin fusion protein comprising, or alternatively consisting of, a Therapeutic protein and a biologically active and/or therapeutically active variant of serum albumin. In some embodiments, the Therapeutic protein portion of the albumin fusion protein is the mature portion of the Therapeutic protein.

In further embodiments, the invention provides an albumin fusion protein comprising, or alternatively consisting of, a biologically active and/or therapeutically active fragment or variant of a Therapeutic protein and a biologically active and/or therapeutically active fragment or variant of serum albumin. In some embodiments, the invention provides an albumin fusion protein comprising, or alternatively consisting of, the mature portion of a Therapeutic protein and the mature portion of serum albumin.

In one embodiment, the albumin fusion protein comprises HA as the N-terminal portion, and a Therapeutic protein as the C-terminal portion. Alternatively, an albumin fusion protein comprising HA as the C-terminal portion, and a Therapeutic protein as the N-terminal portion may also be used.

In other embodiments, the albumin fusion protein has a Therapeutic protein fused to both the N-terminus and the C-terminus of albumin. In one embodiment, the Therapeutic proteins fused at the N- and C-termini are the same Therapeutic proteins. In another embodiment, the Therapeutic proteins fused at the N- and C-termini are different Therapeutic proteins. In another embodiment, the Therapeutic proteins fused at the N- and C-termini are different Therapeutic proteins which may be used to treat or prevent the same disease, disorder, or condition. In another embodiment, the Therapeutic proteins fused at the N- and C-termini are different Therapeutic proteins which may be used to treat or prevent diseases or disorders which are known in the art to commonly occur in patients simultaneously.

In addition to albumin fusion protein in which the albumin portion is fused N-terminal and/or C-terminal of the Therapeutic protein portion, albumin fusion proteins of the invention may also be produced by inserting the Therapeutic protein or peptide of interest into an internal region of HA. For instance, within the protein sequence of the HA molecule a number of loops or turns exist between the end and beginning of α-helices, which are stabilized by disulphide bonds. The loops, as determined from the crystal structure of HA (PDB identifiers 1AO6, 1BJ5, 1BKE, 1BM0, 1E7E to 1E7I and 1UOR) for the most part extend away from the body of the molecule. These loops are useful for the insertion, or internal fusion, of therapeutically active peptides, particularly those requiring a secondary structure to be functional, or Therapeutic proteins, to essentially generate an albumin molecule with specific biological activity.

Loops in human albumin structure into which peptides or polypeptides may be inserted to generate albumin fusion proteins of the invention include: Va154-Asn61, Thr76-Asp89, Ala92-Glu100, Gln170-Ala176, His247-Glu252, Glu266-Glu277, Glu280-His288, Ala362-Glu368, Lys439-Pro447, Val462-Lys475, Thr478-Pro486, and Lys560-Thr566. In other embodiments, peptides or polypeptides are inserted into the Val54-Asn61, Gln170-Ala176, and/or Lys560-Thr566 loops of mature human albumin (SEQ ID NO:18).

Peptides to be inserted may be derived from either phage display or synthetic peptide libraries screened for specific biological activity or from the active portions of a molecule with the desired function. Additionally, random peptide libraries may be generated within particular loops or by insertions of randomized peptides into particular loops of the HA molecule and in which all possible combinations of amino acids are represented.

Such library(s) could be generated on HA or domain fragments of HA by one of the following methods:

(a) randomized mutation of amino acids within one or more peptide loops of HA or HA domain fragments. Either one, more or all the residues within a loop could be mutated in this manner;

(b) replacement of, or insertion into one or more loops of HA or HA domain fragments (i.e., internal fusion) of a randomized peptide(s) of length Xₙ (where X is an amino acid and n is the number of residues;

(c) N-, C- or N- and C-terminal peptide/protein fusions in addition to (a) and/or (b).

The HA or HA domain fragment may also be made multifunctional by grafting the peptides derived from different screens of different loops against different targets into the same HA or HA domain fragment.

Peptides inserted into a loop of human serum albumin are Therapeutic protein peptides or peptide fragments or peptide variants thereof. More particulary, the invention encompasses albumin fusion proteins which comprise peptide fragments or peptide variants at least 7 at least 8, at least 9, at least 10, at least 11, at least 12, at least 13, at least 14, at least 15, at least 20, at least 25, at least 30, at least 35, or at least 40 amino acids in length inserted into a loop of human serum albumin. The invention also encompasses albumin fusion proteins which comprise peptide fragments or peptide variants at least 7 at least 8, at least 9, at least 10, at least 11, at least 12, at least 13, at least 14, at least 15, at least 20, at least 25, at least 30, at least 35, or at least 40 amino acids fused to the N-terminus of human serum albumin. The invention also encompasses albumin fusion proteins which comprise peptide fragments or peptide variants at least 7 at least 8, at least 9, at least 10, at least 11, at least 12, at least 13, at least 14, at least 15, at least 20, at least 25, at least 30, at least 35, or at least 40 amino acids fused to the C-terminus of human serum albumin.

Generally, the albumin fusion proteins of the invention may have one HA-derived region and one Therapeutic protein-derived region. Multiple regions of each protein, however, may be used to make an albumin fusion protein of the invention. Similarly, more than one Therapeutic protein may be used to make an albumin fusion protein of the invention. For instance, a Therapeutic protein may be fused to both the N- and C-terminal ends of the HA. In such a configuration, the Therapeutic protein portions may be the same or different Therapeutic protein molecules. The structure of bifunctional albumin fusion proteins may be represented as: X-HA-Y or Y-HA-X or X-Y-HA or HA-X-Y or HA-X-Y-HA or HA-Y-X-HA or HA-X-X-HA or HA-Y-Y-HA or HA-X-HA-Y or X-HA-Y-HA or multiple combinations or inserting X and/or Y within the HA-sequence at any location.

Bi- or multi-functional albumin fusion proteins may be prepared in various ratios depending on function, half-life etc.

Bi- or multi-functional albumin fusion proteins may also be prepared to target the Therapeutic protein portion of a fusion to a target organ or cell type via protein or peptide at the opposite terminus of HA.

As an alternative to the fusion of known therapeutic molecules, the peptides could be obtained by screening libraries constructed as fusions to the N-, C- or N- and C-termini of HA, or domain fragment of HA, of typically 6, 8, 12, 20 or 25 or Xₙ (where X is an amino acid (aa) and n equals the number of residues) randomized amino acids, and in which all possible combinations of amino acids were represented. A particular advantage of this approach is that the peptides may be selected in situ on the HA molecule and the properties of the peptide would therefore be as selected for rather than, potentially, modified as might be the case for a peptide derived by any other method then being attached to HA.

Additionally, the albumin fusion proteins of the invention may include a linker peptide between the fused portions to provide greater physical separation between the moieties and thus maximize the accessibility of the Therapeutic protein portion, for instance, for binding to its cognate receptor. The linker peptide may consist of amino acids such that it is flexible or more rigid.

Therefore, as described above, the albumin fusion proteins of the invention may have the following formula R2-R1; R1-R2; R2-R1-R2; R2-L-R1-L-R2; R1-L-R 2; R2-L-R1; or R1-L-R2-L-R1, wherein R1 is at least one Therapeutic protein, peptide or polypeptide sequence (including fragments or variants thereof), and not necessarily the same Therapeutic protein, L is a linker and R2 is a serum albumin sequence (including fragments or variants thereof) Exemplary linkers include (GGGGS)_{N} (SEQ ID NO:3) or (GGGS)_{N} (SEQ ID NO:4) or (GGS)_{N}, wherein N is an integer greater than or equal to 1 and wherein G represents glycine and S represents serine. When R1 is two or more Therapeutic proteins, peptides or polypeptide sequence, these sequences may optionally be connected by a linker.

In other embodiments, albumin fusion proteins of the invention comprising a Therapeutic protein have extended shelf-life or in vivo half-life or therapeutic activity compared to the shelf-life or in vivo half-life or therapeutic activity of the same Therapeutic protein when not fused to albumin. Shelf-life typically refers to the time period over which the therapeutic activity of a Therapeutic protein in solution or in some other storage formulation, is stable without undue loss of therapeutic activity. Many of the Therapeutic proteins are highly labile in their unfused state. As described below, the typical shelf-life of these Therapeutic proteins is markedly prolonged upon incorporation into the albumin fusion protein of the invention.

Albumin fusion proteins of the invention with “prolonged” or “extended” shelf-life exhibit greater therapeutic activity relative to a standard that has been subjected to the same storage and handling conditions. The standard may be the unfused full-length Therapeutic protein. When the Therapeutic protein portion of the albumin fusion protein is an analog, a variant, or is otherwise altered or does not include the complete sequence for that protein, the prolongation of therapeutic activity may alternatively be compared to the unfused equivalent of that analog, variant, altered peptide or incomplete sequence. As an example, an albumin fusion protein of the invention may retain greater than about 100% of the therapeutic activity, or greater than about 105%, 110%, 120%, 130%, 150% or 200% of the therapeutic activity of a standard when subjected to the same storage and handling conditions as the standard when compared at a given time point. However, it is noted that the therapeutic activity depends on the Therapeutic protein's stability, and may be below 100%.

Shelf-life may also be assessed in terms of therapeutic activity remaining after storage, normalized to therapeutic activity when storage began. Albumin fusion proteins of the invention with prolonged or extended shelf-life as exhibited by prolonged or extended therapeutic activity may retain greater than about 50% of the therapeutic activity, about 60%, 70%, 80%, or 90% or more of the therapeutic activity of the equivalent unfused Therapeutic protein when subjected to the same conditions.

Therapeutic Proteins

As stated above, an albumin fusion protein of the invention comprises at least a fragment or variant of a Therapeutic protein and at least a fragment or variant of human serum albumin, which are associated with one another by genetic fusion.

As used herein, “Therapeutic protein” refers to an angiogenesis inhibiting peptide, such as endostatin (including restin, arresten, canstatin and tumstatin), or fragments or variants thereof, having one or more therapeutic and/or biological activities; angiostatin or fragments or variants thereof, having one or more therapeutic and/or biological activities, alphastatin or fragments or variants thereof, having one or more therapeutic and/or biological activities, kringle 5 or fragments or variants thereof, having one or more therapeutic and/or biological activities, anti-thrombin III or fragments or variants thereof, having one or more therapeutic and/or biological activities. Thus an albumin fusion protein of the invention may contain at least a fragment or variant of a Therapeutic protein. Additionally, the term “Therapeutic protein” may refer to the endogenous or naturally occurring correlate of a Therapeutic protein. Variants include mutants, analogs, and mimetics, as well as homologs, including the endogenous or naturally occurring correlates of a Therapeutic protein.

By a polypeptide displaying a “therapeutic activity” or a protein that is “therapeutically active” is meant a polypeptide that possesses one or more known biological and/or therapeutic activities associated with a Therapeutic protein such as one or more of the Therapeutic proteins described herein or otherwise known in the art. As a non-limiting example, a “Therapeutic protein” is a protein that is useful to treat, prevent or ameliorate a disease, condition or disorder.

As used herein, “therapeutic activity” or “activity” may refer to an activity whose effect is consistent with a desirable therapeutic outcome in humans, or to desired effects in non-human mammals or in other species or organisms. Therapeutic activity may be measured in vivo or in vitro. For example, a desirable effect may be assayed in cell culture. Such in vitro or cell culture assays are commonly available for many Therapeutic proteins as described in the art.

Examples of useful assays include, but are not limited to, those described in references and publications of Table 1, specifically incorporated by reference herein, and those described in the Examples herein. The anti-angiogenesis or anti-tumor activity exhibited by the fusion proteins of the invention may be measured, for example, by easily performed in vitro assays, such as those described herein, which can test the fusion proteins' ability to inhibit angiogenesis, or their ability to inhibit tumor growth or proliferation. Using these assays, such parameters as the relative anti-angiogenic or anti-tumor activity that the fusion proteins exhibit against a given tumor can be determined.

Therapeutic proteins corresponding to a Therapeutic protein portion of an albumin fusion protein of the invention may be modified by the attachment of one or more oligosaccharide groups. The modification, referred to as glycosylation, can dramatically affect the physical properties of proteins and can be important in protein stability, secretion, and localization. Such modifications are described in detail in U.S. Provisional Application Ser. No. 60/355,547 and WO 01/79480, which are incorporated herein by reference.

Therapeutic proteins corresponding to a Therapeutic protein portion of an albumin fusion protein of the invention, as well as analogs and variants thereof, may be modified so that glycosylation at one or more sites is altered as a result of manipulation(s) of their nucleic acid sequence, by the host cell in which they are expressed, or due to other conditions of their expression. For example, glycosylation isomers may be produced by abolishing or introducing glycosylation sites, e.g., by substitution or deletion of amino acid residues, such as substitution of glutamine for asparagine, or unglycosylated recombinant proteins may be produced by expressing the proteins in host cells that will not glycosylate them, e.g. in E. coli or glycosylation-deficient yeast. Examples of these approaches are described in more detail in U.S. Provisional Application Ser. No. 60/355,547 and WO 01/79480, which are incorporated by reference, and are known in the art.

Table 1 provides a non-exhaustive list of Therapeutic proteins that correspond to a Therapeutic protein portion of an albumin fusion protein of the invention. The “Therapeutic Protein X” column discloses Therapeutic protein molecules followed by parentheses containing scientific and brand names that comprise, or alternatively consist of, that Therapeutic protein molecule or a fragment or variant thereof. “Therapeutic protein X” as used herein may refer either to an individual Therapeutic protein molecule (as defined by the amino acid sequence obtainable from the CAS and Genbank accession numbers), or to the entire group of Therapeutic proteins associated with a given Therapeutic protein molecule disclosed in this column. The information associated with each of these entries are each incorporated by reference in their entireties, particularly with respect to the amino acid sequences described therein. The “PCT/Patent Reference” column provides U.S. patent numbers, or PCT International Publication Numbers corresponding to patents and/or published patent applications that describe the Therapeutic protein molecule. Each of the patents and/or published patent applications cited in the “PCT/Patent Reference” column are herein incorporated by reference in their entireties. In particular, the amino acid sequences of the specified polypeptide set forth in the sequence listing of each cited “PCT/Patent Reference”, the variants of these amino acid sequences (mutations, fragments, etc.) set forth, for example, in the detailed description of each cited “PCT/Patent Reference”, the therapeutic indications set forth, for example, in the detailed description of each cited “PCT/Patent Reference”, and the activity assays for the specified polypeptide set forth in the detailed description, and more particularly, the examples of each cited “PCT/Patent Reference” are incorporated herein by reference. The “Biological activity” column describes Biological activities associated with the Therapeutic protein molecule. Each of the references cited in the “Relevant Information” column are herein incorporated by reference in their entireties, particularly with respect to the description of the respective activity assay described in the reference (see Methods section, for example) for assaying the corresponding biological activity. The “Preferred Indication Y” column describes disease, disorders, and/or conditions that may be treated, prevented, diagnosed, or ameliorated by Therapeutic protein X or an albumin fusion protein of the invention comprising a Therapeutic protein X portion.

TABLE 1

| Therapeutic | PCT/Patent | | | Preferred |
|---|---|---|---|---|
| Protein X | Reference | Biological Activity | Relevant Publications | Indication Y |
| Endostatin | U.S. Pat. No. 5854205, | These are | Sim et al. (2000) Cancer and | Solid tumors and |
| | WO9715666 | antiangiogenic peptides | Metastasis Reviews 19: 181-190, | cancer. |
| | | that suppress the | Dhanabal (1999) Cancer Research | |
| | | growth of tumors | 59: 189-197 | |
| Angiostatin | U.S. Pat. No. 5885795, | These are | Sim et al. (2000) Cancer and | Solid tumors and |
| | U.S. Pat. No. 5792845 | antiangiogenic peptides | Metastasis Reviews 19: 181-190 | cancer |
| | | that suppress the | | |
| | | growth of tumors | | |
| Kringle 5 | U.S. Pat. No. 5854221 | These are | Cao et al. (1996) J. Biological | Solid tumors and |
| | | antiangiogenic peptides | Chemistry 271, 46: 29461-29467; | cancer |
| | | that suppress the | Cao et al. (1997) J. Biological | |
| | | growth of tumors | Chemistry 272, 36: 22924-22928; | |
| | | | Lu et al. (1999) Biochem. | |
| | | | Biophysical Research | |
| | | | Communications, 258, 668-673 | |

In various embodiments, the albumin fusion proteins of the invention are capable of a therapeutic activity and/or biologic activity corresponding to the therapeutic activity and/or biologic activity of the Therapeutic protein corresponding to the Therapeutic protein portion of the albumin fusion protein listed in the corresponding row of Table 1. (See, e.g., the “Biological Activity” and “Therapeutic Protein X” columns of Table 1.) In further embodiments, the therapeutically active protein portions of the albumin fusion proteins of the invention are fragments or variants of the reference sequence and are capable of the therapeutic activity and/or biologic activity of the corresponding Therapeutic protein disclosed in “Biological Activity” column of Table 1.

Polypeptide and Polynucleotide Fragments and Variants

Fragments

The present invention is further directed to fragments of the Therapeutic proteins described in Table 1, albumin proteins, and/or albumin fusion proteins of the invention.

Even if deletion of one or more amino acids from the N-terminus of a protein results in modification or loss of one or more biological functions of the Therapeutic protein, albumin protein, and/or albumin fusion protein, other Therapeutic activities and/or functional activities (e.g., biological activities, ability to multimerize, ability to bind a ligand) may still be retained. For example, the ability of polypeptides with N-terminal deletions to induce and/or bind to antibodies which recognize the complete or mature forms of the polypeptides generally will be retained when less than the majority of the residues of the complete polypeptide are removed from the N-terminus. Whether a particular polypeptide lacking N-terminal residues of a complete polypeptide retains such immunologic activities can readily be determined by routine methods described herein and otherwise known in the art. It is not unlikely that a mutein with a large number of deleted N-terminal amino acid residues may retain some biological or immunogenic activities. In fact, peptides composed of as few as six amino acid residues may often evoke an immune response.

Accordingly, fragments of a Therapeutic protein corresponding to a Therapeutic protein portion of an albumin fusion protein of the invention, include the full length protein as well as polypeptides having one or more residues deleted from the amino terminus of the amino acid sequence of the reference polypeptide (e.g., a Therapeutic protein as disclosed in Table 1). Polynucleotides encoding these polypeptides are also encompassed by the invention.

In addition, fragments of serum albumin polypeptides corresponding to an albumin protein portion of an albumin fusion protein of the invention, include the full length protein as well as polypeptides having one or more residues deleted from the amino terminus of the amino acid sequence of the reference polypeptide (i.e., serum albumin). Polynucleotides encoding these polypeptides are also encompassed by the invention.

Moreover, fragments of albumin fusion proteins of the invention, include the full length albumin fusion protein as well as polypeptides having one or more residues deleted from the amino terminus of the albumin fusion protein. Polynucleotides encoding these polypeptides are also encompassed by the invention.

Also as mentioned above, even if deletion of one or more amino acids from the N-terminus or C-terminus of a reference polypeptide (e.g., a Therapeutic protein and/or serum albumin protein) results in modification or loss of one or more biological functions of the protein, other functional activities (e.g., biological activities, ability to multimerize, ability to bind a ligand) and/or Therapeutic activities may still be retained. For example the ability of polypeptides with C-terminal deletions to induce and/or bind to antibodies which recognize the complete or mature forms of the polypeptide generally will be retained when less than the majority of the residues of the complete or mature polypeptide are removed from the C-terminus. Whether a particular polypeptide lacking the N-terminal and/or C-terminal residues of a reference polypeptide retains Therapeutic activity can readily be determined by routine methods described herein and/or otherwise known in the art.

The present invention further provides polypeptides having one or more residues deleted from the carboxy terminus of the amino acid sequence of a Therapeutic protein corresponding to a Therapeutic protein portion of an albumin fusion protein of the invention (e.g., a Therapeutic protein referred to in Table 1). Polynucleotides encoding these polypeptides are also encompassed by the invention.

In addition, the present invention provides polypeptides having one or more residues deleted from the carboxy terminus of the amino acid sequence of an albumin protein corresponding to an albumin protein portion of an albumin fusion protein of the invention (e.g., serum albumin). Polynucleotides encoding these polypeptides are also encompassed by the invention.

Moreover, the present invention provides polypeptides having one or more residues deleted from the carboxy terminus of an albumin fusion protein of the invention. Polynucleotides encoding these polypeptides are also encompassed by the invention.

In addition, any of the above described N- or C-terminal deletions can be combined to produce a N- and C-terminal deleted reference polypeptide (e.g., a Therapeutic protein referred to in Table 1, or serum albumin (e.g., SEQ ID NO: 18), or an albumin fusion protein of the invention). The invention also provides polypeptides having one or more amino acids deleted from both the amino and the carboxyl termini. Polynucleotides encoding these polypeptides are also encompassed by the invention.

The present application is also directed to proteins containing polypeptides at least 60%, 80%, 85%, 90%, 95%, 96%, 97%, 98% or 99% identical to a reference polypeptide sequence (e.g., a Therapeutic protein, serum albumin protein or an albumin fusion protein of the invention) set forth herein, or fragments thereof. In some embodiments, the application is directed to proteins comprising polypeptides at least 80%, 85%, 90%, 95%, 96%, 97%, 98% or 99% identical to reference polypeptides having the amino acid sequence of N- and C-terminal deletions as described above. Polynucleotides encoding these polypeptides are also encompassed by the invention.

Other polypeptide fragments of the invention are fragments comprising, or alternatively, consisting of, an amino acid sequence that displays a Therapeutic activity and/or functional activity (e.g. biological activity) of the polypeptide sequence of the Therapeutic protein or serum albumin protein of which the amino acid sequence is a fragment.

Other polypeptide fragments are biologically active fragments. Biologically active fragments are those exhibiting activity similar, but not necessarily identical, to an activity of the polypeptide of the present invention. The biological activity of the fragments may include an improved desired activity, or a decreased undesirable activity.

Variants

“Variant” refers to a polynucleotide or nucleic acid differing from a reference nucleic acid or polypeptide, but retaining essential properties thereof. Generally, variants are overall closely similar, and, in many regions, identical to the reference nucleic acid or polypeptide.

As used herein, “variant”, refers to a Therapeutic protein portion of an albumin fusion protein of the invention, albumin portion of an albumin fusion protein of the invention, or albumin fusion protein differing in sequence from a Therapeutic protein (e.g. see “therapeutic” column of Table 1), albumin protein, and/or albumin fusion protein of the invention, respectively, but retaining at least one functional and/or therapeutic property thereof (e.g., a therapeutic activity and/or biological activity as disclosed in the “Biological Activity” column of Table 1) as described elsewhere herein or otherwise known in the art. Generally, variants are overall very similar, and, in many regions, identical to the amino acid sequence of the Therapeutic protein corresponding to a Therapeutic protein portion of an albumin fusion protein of the invention, albumin protein corresponding to an albumin protein portion of an albumin fusion protein of the invention, and/or albumin fusion protein of the invention. Nucleic acids encoding these variants are also encompassed by the invention.

The present invention is also directed to proteins which comprise, or alternatively consist of, an amino acid sequence which is at least 60%, 80%, 85%, 90%, 95%, 96%, 97%, 98%, 99% or 100%, identical to, for example, the amino acid sequence of a Therapeutic protein corresponding to a Therapeutic protein portion of an albumin fusion protein of the invention (e.g., an amino acid sequence disclosed in a reference in Table 1, or fragments or variants thereof), albumin proteins (e.g., SEQ ID NO:18 or fragments or variants thereof) corresponding to an albumin protein portion of an albumin fusion protein of the invention, and/or albumin fusion proteins of the invention. Fragments of these polypeptides are also provided (e.g., those fragments described herein). Further polypeptides encompassed by the invention are polypeptides encoded by polynucleotides which hybridize to the complement of a nucleic acid molecule encoding an amino acid sequence of the invention under stringent hybridization conditions (e.g., hybridization to filter bound DNA in 6× Sodium chloride/Sodium citrate (SSC) at about 45 degrees Celsius, followed by one or more washes in 0.2×SSC, 0.1% SDS at about 50-65 degrees Celsius), under highly stringent conditions (e.g., hybridization to filter bound DNA in 6× sodium chloride/Sodium citrate (SSC) at about 45 degrees Celsius, followed by one or more washes in 0.1×SSC, 0.2% SDS at about 68 degrees Celsius), or under other stringent hybridization conditions which are known to those of skill in the art (see, for example, Ausubel, F. M. et al., eds., 1989 Current protocol in Molecular Biology, Green publishing associates, Inc., and John Wiley & Sons Inc., New York, at pages 6.3.1-6.3.6 and 2.10.3). Polynucleotides encoding these polypeptides are also encompassed by the invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least 60%, 80%, 85%, 90%, 95%, 96%, 97%, 98% or 99% identical to, for instance, the amino acid sequence of an albumin fusion protein of the invention or a fragment thereof (such as the Therapeutic protein portion of the albumin fusion protein or the albumin portion of the albumin fusion protein), can be determined conventionally using known computer programs. Such programs and methods of using them are described, e.g., in U.S. Provisional application Ser. No. 60/355,547 and WO 01/79480 (pp. 41-43), which are incorporated by reference herein, and are well known in the art.

The polynucleotide variants of the invention may contain alterations in the coding regions, non-coding regions, or both. Polynucleotide variants include those containing alterations which produce silent substitutions, additions, or deletions, but do not alter the properties or activities of the encoded polypeptide. Such nucleotide variants may be produced by silent substitutions due to the degeneracy of the genetic code. Polypeptide variants include those in which less than 50, less than 40, less than 30, less than 20, less than 10, or 5-50, 5-25, 5-10, 1-5, or 1-2 amino acids are substituted, deleted, or added in any combination. Polynucleotide variants can be produced for a variety of reasons, e.g., to optimize codon expression for a particular host (change codons in the human mRNA to those preferred by a microbial host, such as, yeast or E. coli).

In another embodiment, a polynucleotide encoding an albumin portion of an albumin fusion protein of the invention is optimized for expression in yeast or mammalian cells. In yet another embodiment, a polynucleotide encoding a Therapeutic protein portion of an albumin fusion protein of the invention is optimized for expression in yeast or mammalian cells. In still another embodiment, a polynucleotide encoding an albumin fusion protein of the invention is optimized for expression in yeast or mammalian cells.

In an alternative embodiment, a codon optimized polynucleotide encoding a Therapeutic protein portion of an albumin fusion protein of the invention does not hybridize to the wild type polynucleotide encoding the Therapeutic protein under stringent hybridization conditions as described herein. In a further embodiment, a codon optimized polynucleotide encoding an albumin portion of an albumin fusion protein of the invention does not hybridize to the wild type polynucleotide encoding the albumin protein under stringent hybridization conditions as described herein. In another embodiment, a codon optimized polynucleotide encoding an albumin fusion protein of the invention does not hybridize to the wild type polynucleotide encoding the Therapeutic protein portin or the albumin protein portion under stringent hybridization conditions as described herein.

In an additional embodiment, polynucleotides encoding a Therapeutic protein portion of an albumin fusion protein of the invention do not comprise, or alternatively consist of, the naturally occurring sequence of that Therapeutic protein. In a further embodiment, polynucleotides encoding an albumin protein portion of an albumin fusion protein of the invention do not comprise, or alternatively consist of, the naturally occurring sequence of albumin protein. In an alternative embodiment, polynucleotides encoding an albumin fusion protein of the invention do not comprise, or alternatively consist of, the naturally occurring sequence of a Therapeutic protein portion or the albumin protein portion.

In an additional embodiment, the Therapeutic protein may be selected from a random peptide library by biopanning, as there will be no naturally occuring wild type polynucleotide.

Naturally occurring variants are called “allelic variants,” and refer to one of several alternate forms of a gene occupying a given locus on a chromosome of an organism. (Genes II, Lewin, B., ed., John Wiley & Sons, New York (1985)). These allelic variants can vary at either the polynucleotide and/or polypeptide level and are included in the present invention. Alternatively, non-naturally occurring variants may be produced by mutagenesis techniques or by direct synthesis.

Using known methods of protein engineering and recombinant DNA technology, variants may be generated to improve or alter the characteristics of the polypeptides of the present invention. For instance, one or more amino acids may be deleted from the N-terminus or C-terminus of the polypeptide of the present invention without substantial loss of biological function. See, e.g., Ron et al., J. Biol. Chem. 268: 2984-2988 (1993) (KGF variants) and Dobeli et al., J. Biotechnology 7:199-216 (1988) (interferon gamma variants).

Moreover, ample evidence demonstrates that variants often retain a biological activity similar to that of the naturally occurring protein (e.g., Gayle et al., J. Biol. Chem. 268:22105-22111 (1993) (IL-1a variants)). Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C-terminal residues of a protein retains such immunogenic activities can readily be determined by routine methods described herein and otherwise known in the art.

Thus, the invention further includes polypeptide variants which have a functional activity (e.g., biological activity and/or therapeutic activity). In further embodiments the invention provides variants of albumin fusion proteins that have a functional activity (e.g., biological activity and/or therapeutic activity, such as that disclosed in the “Biological Activity” column in Table 1) that corresponds to one or more biological and/or therapeutic activities of the Therapeutic protein corresponding to the Therapeutic protein portion of the albumin fusion protein. Such variants include deletions, insertions, inversions, repeats, and substitutions selected according to general rules known in the art so as have little effect on activity.

In other embodiments, the variants of the invention have conservative substitutions. By “conservative substitutions” is intended swaps within groups such as replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

Guidance concerning how to make phenotypically silent amino acid substitutions is provided, for example, in Bowie et al., “Deciphering the Message in Protein Sequences: Tolerance to Amino Acid Substitutions,” Science 247:1306-1310 (1990), wherein the authors indicate that there are two main strategies for studying the tolerance of an amino acid sequence to change.

As the authors state, proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved. Moreover, tolerated conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

Besides conservative amino acid substitution, variants of the present invention include (i) polypeptides containing substitutions of one or more of the non-conserved amino acid residues, where the substituted amino acid residues may or may not be one encoded by the genetic code, or (ii) polypeptides containing substitutions of one or more of the amino acid residues having a substituent group, or (iii) polypeptides which have been fused with or chemically conjugated to another compound, such as a compound to increase the stability and/or solubility of the polypeptide (for example, polyethylene glycol), (iv) polypeptide containing additional amino acids, such as, for example, an IgG Fc fusion region peptide. Such variant polypeptides are deemed to be within the scope of those skilled in the art from the teachings herein.

For example, polypeptide variants containing amino acid substitutions of charged amino acids with other charged or neutral amino acids may produce proteins with improved characteristics, such as less aggregation. Aggregation of pharmaceutical formulations both reduces activity and increases clearance due to the aggregate's immunogenic activity. See Pinckard et al., Clin. Exp. Immunol. 2:331-340 (1967); Robbins et al., Diabetes 36: 838-845 (1987); Cleland et al., Crit. Rev. Therapeutic Drug Carrier Systems 10:307-377 (1993).

In specific embodiments, the polypeptides of the invention comprise, or alternatively, consist of, fragments or variants of the amino acid sequence of a Therapeutic protein described herein and/or human serum albumin, and/or albumin fusion protein of the invention, wherein the fragments or variants have 1-5, 5-10, 5-25, 5-50, 10-50 or 50-150, amino acid residue additions, substitutions, and/or deletions when compared to the reference amino acid sequence. In certain embodiments, the amino acid substitutions are conservative. Nucleic acids encoding these polypeptides are also encompassed by the invention.

The polypeptide of the present invention can be composed of amino acids joined to each other by peptide bonds or modified peptide bonds, i.e., peptide isosteres, and may contain amino acids other than the 20 gene-encoded amino acids. The polypeptides may be modified by either natural processes, such as post-translational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristylation, oxidation, pegylation, proteolytic processing, phosphorylation, prenylation, racemization, selenoylation, sulfation, transfer-RNA mediated addition of amino acids to proteins such as arginylation, and ubiquitination.

Furthermore, chemical entities may be covalently attached to the albumin fusion proteins of the invention to enhance or modulate a specific functional or biological activity such as by methods disclosed in Current Opinions in Biotechnology, 10:324 (1999).

Furthermore, targetting entities may be covalently attached to the albumin fusion proteins of the invention to target a specific functional or biological activity to certain cell or stage specific types, tissue types or anatomical structures. By directing albumin fusion proteins of the invention the action of the agent may be localised. Further, such targeting may enable the dosage of the albumin fusion proteins of the invention required to be reduced since, by accumulating the albumin fusion proteins of the invention at the required site, a higher localised concentration may be achieved. Albumin fusion proteins of the invention can be conjugated with a targeting portion by use of cross-linking agents as well as by recombinant DNA techniques whereby the nucleotide sequence encoding the albumin fusion proteins of the invention, or a functional portion of it, is cloned adjacent to the nucleotide sequence of the ligand when the ligand is a protein, and the conjugate expressed as a fusion protein. The targeting agent can be any monoclonal antibody, or active portion thereof, eg Fab or F(ab′)₂ fragment, a ligand (natural or synthetic) recognised by an endothelial cell surface receptor or a functional portion thereof, or any other agent which interacts with protein or structures of the endothelial cell.

The active antibody portions, eg Fab or F(ab′)₂ fragments of antibodies, will retain antigen/target binding but have low non-specific binding. Fab or F(ab′)₂ fragments may be obtained by protease digestion, for example using immobilised Protein A and pepsin/papain digestion using ImmunoPure Fab and ImmunoPure F(ab′)₂ preparation kits (Pierce). Other active portions of antibodies may be obtained by reduction of the antibodies or antibody fragments into separate heavy and light chains.

Molecules targeted by albumin fusion proteins of the invention/antibody conjugates or gene fusions can be endothelial cell surface molecules, extracellular matrix components, for example collagen, fibronectin or laminin, or other blood vessel wall structures. Examples of monoclonal antibodies raised to endothelial surface antigens are Tük3 (Dako) and QBend10 (Serotec) which recognise CD34, a glycosylated endothelial cell surface transmembrane protein. Other monoclonal antibodies raised to endothelial cell surface antigens include 9G11, JC70, and By126 (British Bio-technology) raised to CD31 (also known as PECAM-1) and ESIVC7 raised to the CD36 antigen, which is the thrombospondin receptor (Kuzu et al (1992) J. Clin. Pathol. 45, 143-148). QBend20, QBend30 and QBend40 (Serotec) are examples of other monoclonal antibodies which recognise endothelial cell surface antigens.

The endothelial cell surface molecules to which the targeting antibodies are raised can be non-specific and recognise a number of different endothelial cell types from different tissues, or can be specific for certain endothelial cell types. Antibody A10-33/1 (Serotec) recognises endothelial cells in metastatic melanomas, H4-7/33 (Serotec) recognises endothelial cells from small capillaries and a wide range of tumour cells, HM15/3 (Serotec) recognises sinusoidal endothelial cells, and 1F/10 (Serotec) binds to a 250 kD surface protein on continuous endothelium. Antibodies raised to antigens involved in haemostasis and inflammation can also be used. Antibody 4D10 (Serotec) and BB11 (Benjamin et al (1990) Biochem. Biophys. Res. Commun. 171, 348-353) recognises ELAM-1 present on endothelial cells in acute inflamed tissues. Antibody 4B9 (Carlo, T. and Harlan, J. (1990) Immunol. Rev. 114, 1-24) recognises the VCAM adhesion protein. Antibody 84H10 (Makgabo, M. et al (1988) Nature 331, 86-88) recognises the ICAM1 adhesion protein. Antibody EN7/58 (Serotec) recognises antigens present on inflamed endothelium and on cells adhering to the endothelial cells. Antibody KG7/30 recognises a FVIII related protein on endothelial surfaces of inflamed tissues and tumours.

The cytokines IL-1 and TNF stimulate cultured endothelial cells to acquire adhesive properties for various peripheral blood leukocytes in vitro (Bevilaqua, M. et al. (1985) J. Clin. Invest. 76, 2003; Schleimer, R. et al (1986) J. Immunol. 136, 649; Lamas, A. et al (1988) J. Immunol. 140, 1500; Bochner, B. et al (1988) J. Clin. Invest. 81, 1355). This adhesiveness is associated with the induction on endothelial cells of a number of adhesive molecules, including ICAM-1, ELAM-1, GMP-140 (also known as PADGEM or CD62) and VCAM-1. These adhesive molecules recognise counter receptors on the surface of the target cell. VCAM-1 recognises an antigen known as VLA-4, also known as CD49d/CD29 and member of the integrin family (Elices, M. et al (1990) Cell 60, 577; Schwartz, B. et al (1990) J. Clin. Invest. 85, 2019). ICAM-1 recognises an antigen known as LFA-1, also known as CD11a/CD18, another member of the integrin family (Martin, S. et al (1987) Cell 51, 813-819 Fujita, H. et al (1991) Biochem. Biophys. Res. Comm. 177, 664-672). ELAM-1 and GMP-140 (GMP-140 is also known as CD62 or PADGEM), recognise an antigen known as Lewis X, also known as CD 15, or sialyl-LewisX (Larsen, E. et al (1990) Cell 63, 467-474; McEver, R. (1991) J. Cell. Biochem. 45, 156-161; Shimizu, Y. et al (1991) Nature 349, 799; Picker, L. et al (1991) Nature 349, 796-798; Polley, M. et al (1991) Proc. Natl. Acad. Sci. USA. 88, 6224-6228; Lowe, J. et al (1990) Cell 63, 475-484; Tiemeyer, M. et al (1991) Proc. Natl. Acad. Sci. USA. 88, 1138-1142).

Monoclonal antibodies to either the receptor expressed on the surface of the endothelial cell or counter receptor on the surface of the responding cell have been shown to block interaction of the components necessary for this cell-cell recognition and were instrumental in establishing the mode of recognition (for references see above).

Thus, one aspect of the invention is the provision of a method of targeting an antiangiogenic peptide to the inside of a cell or at cell structures in a mammal by administering a fusion protein of the invention to a mammal.

Another aspect of this invention provides a conjugate of albumin fusion proteins of the invention and a moiety which specifically binds endothelial cells.

Albumin fusion proteins of the invention can be conjugated, by crosslinking or by recombinant DNA techniques, to natural or synthetic ligands which interact with receptors on the endothelial cell surface. Such ligands include growth factors, for example vascular permeability factor (Gitay-Goren, H. et al (1992) J. Biol. Chem. 267, 6093-6098; Bikfalin, A. et al (1991) J. Cell. Phys. 149, 50-59; Tischer, E. et al (1991) 266, 11947-11954; Conn, G. et al (1990) PNAS 87, 2628-2632; Keck, P. et al (1989) Science 246, 1309-1312; Leung, D. W. et al (1989) Science 246, 1306-1309); platelet-derived growth factor (Beitz, J. et al (1991) PNAS 88, 2021-2025); and well as other biomolecules such as transferrin and urokinase (Haddock, R. et al (1991) J. Biol. Chem. 266, 21466-21473). The ligand domain of the conjugates will be recognised by the endothelial cell surface receptor for that ligand and will target the albumin fusion proteins of the invention to the endothelium.

Albumin fusion proteins of the invention can also be directed toward a specific adhesion molecule by cross-linking the agent to the counter receptor for that adhesion molecule. In the example of ELAM-1 mediated adhesion, the counter receptor is a carbohydrate determinant known as Lewis-X or sialylated Lewis-X. Synthetic carbohydrates with this terminal structure (Kameyama, A. et al (1991) Carbohydrate. Res. 209, C1-C4) or purified from natural sources, for example LNFIII (Calbiochem), are available. The terminal Lewis-X or sialyl Lewis-X determinant can be cross-linked to free sulphydryl groups within the albumin fusion proteins of the invention. This allows specific targeting of the agent to endothelial cells presenting the ELAM-1 adhesion molecule.

This moiety may be a monoclonal antibody to endothelial cell surface receptors such as ICAM-1, ELAM-1, GMP-140 or VCAM-1. Alternatively, this moiety may be the counter receptor itself, or a functional portion thereof. Fusion may be achieved by i) chemical cross linking of the moiety, be it a monoclonal antibody or the counter receptor, by techniques known in the art, or ii) by recombinant DNA technology whereby the moiety, when it is a single polypeptide chain, is expressed as a gene fusion with the agent in a suitable host.

A number of cell- or stage-specific antibodies have been described. These include, for example antibodies to endothelial cell adhesion, molecules, including antibody BB11 (anti-ELAM, Benjamin, C. et al (1990), Biochem. Biophys. Res. Commun. 171, 348-353), antibody 4B9 (anti-VCAM, Carlo, T. and Harlan, J. (1990) Immunol. Rev. 114, 1-24) and antibody 84H10 (anti-ICAM1, Makgobo, M. et al (1988) Nature 331, 86-88). These antibodies, or antibodies like them, can be covalently joined to albumin fusion proteins of the invention. This can be achieved by gene fusion whereby the nucleotide sequence encoding the albumin fusion proteins of the invention is spliced into the genes encoding either the heavy or light chain of the antibody, or as a scFv. Alternatively the agent can be covalently cross-linked to the antibody via one of a number of bi-functional cross-linking reagent for example disuccinimidyl suberate (DSS); bis (sulfosuccinimidyl) suberate (BS³); dimethyl adipimidate-2 HCl (DMA); dimethyl pimelimidate-2 HCl (DMP); dimethyl suberimidate-2 HCl (DMS); bismaleimidohexane (BMH); m-maleimidobenzoyl-N-hydroxysuccinimide ester (MBS); m-maleimido-benzoyl-N-hydroxysulfosuccinimide ester (sulfo-MBS); succinimidyl 4-(p-maleimidophenyl)butyrate (SMPB); sulfosuccinimidyl 4-(p-maleimidophenyl)butyrate (sulfo-SMPB); N-succinimidyl (4-iodoacetyl) aminobenzoate (SIAB); sulfosuccinimidyl (4-iodoacetyl) aminobenzoate (sulfo-SIAB); succinimidyl 4-(N-maleimidomethyl)cyclohexane-1-carboxylate (SMCC); sulfosuccinimidyl 4-(N-maleimidomethyl) cyclohexane-1-carboxylate (Sulfo-SMCC) or 1,5-difluoro-2,4-dinitrobenzene (DFDNB), (Pierce).

Antibodies recognising antigens related to malignant transformation and angiogenesis can also be used: for example EN2/3 (Serotec) recognises an antigen characteristic of malignant transformed endothelial cells; EN7/44 (Serotec) recognises an angiogenesis related antigen present on proliferating, migrating and budding endothelial cells; and H3-5/47 recognises endothelial cells in angioblasts, angiomas, angiosarcomas and perivascular cells in psoriasis and arthritic tissues.

Alternatively, the entity which is recognised by the targeting portion may be a suitable entity which is specifically expressed by tumour cells, which entity is not expressed, or at least not with such frequency, in cells into which one does not wish to target the albumin fusion proteins of the invention. The entity which is recognised will often be an antigen. Examples of antigens include those listed in Table X below. Monoclonal antibodies which will bind specifically to many of these antigens are already known, but in any case, with today's techniques in relation to monoclonal antibody technology, antibodies can be prepared to most antigens. The antigen-specific portion may be an entire antibody (usually, for convenience and specificity, a monoclonal antibody), a part or parts thereof (for example an Fab fragment, F(ab′)₂, or “minimum recognition unit”) or a synthetic antibody or part thereof. A compound comprising only part of an antibody may be advantageous by virtue of being less likely to undergo non-specific binding due to the F_{c} part. Suitable monoclonal antibodies to selected antigens may be prepared by known techniques, for example those disclosed in “Monoclonal Antibodies: A manual of techniques”, H. Zola (CRC Press, 1988) and in “Monoclonal Hybridoma Antibodies: Techniques and Applications”, J. G. R. Hurrell (CRC Press, 1982). All references mentioned in this specification are incorporated herein by reference. Bispecific antibodies may be prepared by cell fusion, by reassociation of monovalent fragments or by chemical cross-linking of whole antibodies, with one part of the resulting bispecific antibody being directed to the cell-specific antigen and the other to the albumin fusion proteins of the invention. The bispecific antibody can be administered bound to the albumin fusion proteins of the invention or it can be administered first, followed by the albumin fusion proteins of the invention. The former is preferred. Methods for preparing bispecific antibodies are disclosed in Corvalan et al (1987) Cancer Immunol. Immunother. 24, 127-132 and 133-137 and 138-143. Bispecific antibodies, chimaeric antibodies and single chain antibodies are discussed generally by Williams in Tibtech, February 1988, Vol. 6, 36-42, Neuberger et al (8th International Biotechnology Symposium, 1988, Part 2, 792-799) and Tan and Morrison (Adv. Drug Delivery Reviews 2, (1988), 129-142). Suitably prepared non-human antibodies can be “humanized” in known ways, for example by inserting the CDR regions of mouse antibodies into the framework of human antibodies. IgG class antibodies are preferred.

TABLE 2

| | | |
|---|---|---|
| Antigen | Antibody | Existing Uses |

1. Tumour Associated Antigens

| | | |
|---|---|---|
| Carcino-embryonic | C46 (Amersham) | Imaging & Therapy of colon/rectum tumours. |
| Antigen 85A12 (Unipath) | | |
| Placental Alkaline | H17E2 (ICRF, Travers | Imaging & Therapy of testicular and ovarian |
| Phosphatase | & Bodmer | cancers. |
| Pan carcinoma | NR-LU-10 (NeoRx | Imaging & Therapy of various carcinomas |
| | Corporation) | incl. small cell lung cancer. |
| Polymorphic Epithelial | HMFG1 (Taylor- | Imaging & Therapy of ovarian cancer, |
| Mucin (Human milk fat | Papadimitriou, ICRF) | pleural effusions. |
| globule) | | |
| β-human Chorionic | W14 | Targeting of enzyme (CPG2) to human |
| Gonadotropin | | xenograft choriocarcinoma in nude mice. |
| | | (Searle et al (1981) Br. J. Cancer 44, |
| | | 137-144). |
| Carbohydrate on | L6 (IgG2a)¹ | Targeting of alkaline phosphatase. |
| Human Carcinomas | | (Senter et al (1988) P.N.A.S. 85, 4842-4846. |
| CD20 Antigen on B | 1F5 (IgG2a)² | Targeting of alkaline phosphatase. |
| Lymphoma (normal and | | (Senter et al (1988) P.N.A.S. 85, 4842-4846. |
| and neoplastic) | | |

2. Immune Cell Antigens

| | | |
|---|---|---|
| Pan T Lymphocyte | OKT-3 (Ortho) | As anti-rejection therapy for kidney |
| Surface Antigen (CD3) | | transplants. |
| B-lymphocyte Surface | RFB4 (Janossy, Royal | Immunotoxin therapy of B cell lymphoma. |
| Antigen (CD22) | Free Hospital) | |
| Pan T lymphocyte | H65 (Bodmer, Knowles | Immunotoxin treatment of Acute Graft |
| Surface Antigen (CD5) | ICRF, Licensed to Xoma | versus Host disease, Rheumatoid Arthritis. |
| | Corp., USA) | |

¹Hellström et al (1986) Cancer Res. 46, 3917-3923
²Clarke et al (1985) P.N.A.S. 82, 1766-1770
Other antigens include alphafoetoprotein, Ca-125 and prostate specific antigen.

If applied to the treatment of CML or ALL, the ligand binding molecules can be monoclonal antibodies against leukaemia-associated antigens. Examples of these are: anti-CALLA (common acute lymphoblastic leukaemia-associated antigen), J5, BA-3, RFB-1, BA-2, SJ-9A4 Du-ALL-1, anti-3-3, anti-3-40, SN1 and CALL2, described in Foon, K. A. et al 1986 Blood 68(1), 1-31, “Review: Immunologic Classification of Leukemia and Lymphoma”. The ligand binding molecules can also be antibodies that identify myeloid cell surface antigens, or antibodies that are reactive with B or T lymphocytes, respectively. Examples of such antibodies are those which identify human myeloid cell surface antigens or those which are reactive with human B or T lymphocytes as described in Foon, K. A. Id. Additional examples are antibodies B43, CD22 and CD19 which are reactive with B lymphocytes can also be used.

Alternatively, the entity which is recognised may or may not be antigenic but can be recognised and selectively bound to in some other way. For example, it may be a characteristic cell surface receptor such as the receptor for melanocyte-stimulating hormone (MSH) which is expressed in high numbers in melanoma cells. The targeting portion may then be a compound or part thereof which specifically binds to the entity in a non-immune sense, for example as a substrate or analogue thereof for a cell-surface enzyme or as a messenger. In the case of melanoma cells, the targeting portion may be MSH itself or a part thereof which binds to the MSH receptor. Such MSH peptides are disclosed in, for example, Al-Obeidi et al (1980) J. Med. Chem. 32, 174. The specificity may be indirect: a first cell-specific antibody may be administered, followed by a conjugate of the invention directed against the first antibody. Preferably, the entity which is recognised is not secreted to any relevant extent into body fluids, since otherwise the requisite specificity may not be achieved.

The targeting portion of the conjugate of this embodiment of the invention may be linked to the albumin fusion proteins of the invention by any of the conventional ways of linking compounds, for example by disulphide, amide or thioether bonds, such as those generally described in Goodchild, supra or in Connolly (1985) Nucl. Acids Res. 13(12), 4485-4502 or in PCT/US85/03312.

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The albumin fusion proteins may also be modified with e.g., but not limited to, chemotherapeutic agents, such as a drug, and/or a detectable label, such as an enzymatic, fluorescent, isotopic and/or affinity label to allow for detection and isolation of the protein. Examples of such modifications are given, e.g., in U.S. Provisional Application Ser. No. 60/355,547 and in WO 01/79480 (pp. 105-106), which are incorporated by reference herein, and are well known in the art.

Functional Activity

“A polypeptide having functional activity” refers to a polypeptide capable of displaying one or more known functional activities associated with the full-length, pro-protein, and/or mature form of a Therapeutic protein. Such functional activities include, but are not limited to, biological activity, antigenicity [ability to bind (or compete with a polypeptide for binding) to an anti-polypeptide antibody], immunogenicity (ability to generate antibody which binds to a specific polypeptide of the invention), ability to form multimers with polypeptides of the invention, and ability to bind to a receptor or ligand for a polypeptide.

“A polypeptide having biological activity” refers to a polypeptide exhibiting activity similar to, but not necessarily identical to, an activity of a Therapeutic protein of the present invention, including mature forms, as measured in a particular biological assay, with or without dose dependency. In the case where dose dependency does exist, it need not be identical to that of the polypeptide, but rather substantially similar to the dose-dependence in a given activity as compared to the polypeptide of the present invention.

In other embodiments, an albumin fusion protein of the invention has at least one biological and/or therapeutic activity associated with the Therapeutic protein (or fragment or variant thereof) when it is not fused to albumin.

The albumin fusion proteins of the invention can be assayed for functional activity (e.g., biological activity) using or routinely modifying assays known in the art, as well as assays described herein. Specifically, albumin fusion proteins may be assayed for functional activity (e.g., biological activity or therapeutic activity) using the assay referenced in the “Relevant Publications” column of Table 1. Additionally, one of skill in the art may routinely assay fragments of a Therapeutic protein corresponding to a Therapeutic protein portion of an albumin fusion protein of the invention, for activity using assays referenced in its corresponding row of Table 1. Further, one of skill in the art may routinely assay fragments of an albumin protein corresponding to an albumin protein portion of an albumin fusion protein of the invention, for activity using assays known in the art and/or as described in the Examples section in U.S. Provisional Application Ser. No. 60/355,547 and WO 01/79480.

In addition, assays described herein (see Examples and Table 1) and otherwise known in the art may routinely be applied to measure the ability of albumin fusion proteins of the present invention and fragments, variants and derivatives thereof to elicit biological activity and/or Therapeutic activity (either in vitro or in vivo) related to either the Therapeutic protein portion and/or albumin portion of the albumin fusion protein of the present invention. Other methods will be known to the skilled artisan and are within the scope of the invention.

Anti-Angiogenesis Activity

The naturally occurring balance between endogenous stimulators and inhibitors of angiogenesis is one in which inhibitory influences predominate. Rastinejad et al., Cell 56:345-355 (1989). In those rare instances in which neovascularization occurs under normal physiological conditions, such as wound heating, organ regeneration, embryonic development, and female reproductive processes, angiogenesis is stringently regulated and spatially and temporally delimited. Under conditions of pathological angiogenesis such as that characterizing solid tumor growth, these regulatory controls fail. Unregulated angiogenesis becomes pathologic and sustains progression of many neoplastic and non-neoplastic diseases. A number of serious diseases are dominated by abnormal neovascularization including solid tumor growth and metastases, arthritis, some types of eye disorders, and psoriasis. See, e.g., reviews by Moses et al., Biotech. 9:630-634 (1991); Folkman et al., N. Engl. J. Med., 333:1757-1763 (1995); Auerbach et al., J. Microvasc. Res. 29:401-411 (1985); Folkman, Advances in Cancer Research, eds. Klein and Weinhouse, Academic Press, New York, pp. 175-203 (1985); Patz, Am. J. Opthalmol. 94:715-743 (1982); and Folkman et al., Science 221:719-725 (1983). In a number of pathological conditions, the process of angiogenesis contributes to the disease state. For example, significant data have accumulated which suggest that the growth of solid tumors is dependent on angiogenesis. Folkman and Klagsbrun, Science 235:442-447 (1987).

The present invention provides for treatment of diseases or disorders associated with neovascularization by administration of fusion proteins of the invention and/or polynucleotides encoding albumin fusion proteins of the invention. Malignant and metastatic conditions which can be treated with the polynucleotides and polypeptides, or agonists or antagonists of the invention include, but are not limited to, malignancies, solid tumors, and cancers described herein and otherwise known in the art (for a review of such disorders, see Fishman et al., Medicine, 2d Ed., J. B. Lippincott Co., Philadelphia (1985)). Thus, the present invention provides a method of treating an angiogenesis-related disease and/or disorder, comprising administering to an individual in need thereof a therapeutically effective amount of an albumin fusion protein of the invention and/or polynucleotides encoding an albumin fusion protein of the invention. For example, fusion proteins of the invention and/or polynucleotides encoding albumin fusion proteins of the invention may be utilized in a variety of additional methods in order to therapeutically treat a cancer or tumor. Cancers which may be treated with fusion proteins of the invention and/or polynucleotides encoding albumin fusion proteins of the invention include, but are not limited to solid tumors, including prostate, lung, breast, ovarian, stomach, pancreas, larynx, esophagus, testes, liver, parotid, biliary tract, colon, rectum, cervix, uterus, endometrium, kidney, bladder, thyroid cancer; primary tumors and metastases; melanomas; glioblastoma; Kaposi's sarcoma; leiomyosarcoma; non-small cell lung cancer; colorectal cancer; advanced malignancies; and blood born tumors such as leukemias. For example, fusion proteins of the invention and/or polynucleotides encoding albumin fusion proteins of the invention may be delivered topically, in order to treat cancers such as skin cancer, head and neck tumors, breast tumors, and Kaposi's sarcoma.

Within yet other aspects, fusion proteins of the invention and/or polynucleotides encoding albumin fusion proteins of the invention may be utilized to treat superficial forms of bladder cancer by, for example, intravesical administration. Albumin fusion proteins of the invention and/or polynucleotides encoding albumin fusion proteins of the invention may be delivered directly into the tumor, or near the tumor site, via injection or a catheter. Of course, as the artisan of ordinary skill will appreciate, the appropriate mode of administration will vary according to the cancer to be treated. Other modes of delivery are discussed herein.

Albumin fusion proteins of the invention and/or polynucleotides encoding albumin fusion proteins of the invention may be useful in treating other disorders, besides cancers, which involve angiogenesis. These disorders include, but are not limited to: benign tumors, for example hemangiomas, acoustic neuromas, neurofibromas, trachomas, and pyogenic granulomas; artheroscleric plaques; ocular angiogenic diseases, for example, diabetic retinopathy, retinopathy of prematurity, macular degeneration, corneal graft rejection, neovascular glaucoma; retrolental fibroplasia, rubeosis, retinoblastoma, uvietis and Pterygia (abnormal blood vessel growth) of the eye; rheumatoid arthritis; psoriasis; delayed wound healing; endometriosis; vasculogenesis; granulations; hypertrophic scars (keloids); nonunion fractures; scleroderma; trachoma; vascular adhesions; myocardial angiogenesis; coronary collaterals; cerebral collaterals; arteriovenous malformations; ischemic limb angiogenesis; Osler-Webber Syndrome; plaque neovascularization; telangiectasia; hemophiliac joints; angiofibroma; fibromuscular dysplasia; wound granulation; Crohn's disease; and atherosclerosis.

For example, within one aspect of the present invention methods are provided for treating hypertrophic scars and keloids, comprising the step of administering albumin fusion proteins of the invention and/or polynucleotides encoding albumin fusion proteins of the invention to a hypertrophic scar or keloid.

Within one embodiment of the present invention fusion proteins of the invention and/or polynucleotides encoding albumin fusion proteins of the invention are directly injected into a hypertrophic scar or keloid, in order to prevent the progression of these lesions. This therapy is of particular value in the prophylactic treatment of conditions which are known to result in the development of hypertrophic scars and keloids (e.g., burns), and is optionally initiated after the proliferative phase has had time to progress (approximately 14 days after the initial injury), but before hypertrophic scar or keloid development. As noted above, the present invention also provides methods for treating neovascular diseases of the eye, including for example, corneal neovascularization, neovascular glaucoma, proliferative diabetic retinopathy, retrolental fibroplasia and macular degeneration.

Moreover, Ocular disorders associated with neovascularization which can be treated with the albumin fusion proteins of the invention and/or polynucleotides encoding albumin fusion proteins of the invention include, but are not limited to: neovascular glaucoma, diabetic retinopathy, retinoblastoma, retrolental fibroplasia, uveitis, retinopathy of prematurity macular degeneration, corneal graft neovascularization, as well as other eye inflammatory diseases, ocular tumors and diseases associated with choroidal or iris neovascularization. See, e.g., reviews by Waltman et al., Am. J. Ophthal. 85:704-710 (1978) and Gartner et al., Surv. Ophthal. 22:291-312 (1978).

Thus, within one aspect of the present invention methods are provided for treating neovascular diseases of the eye such as corneal neovascularization (including corneal graft neovascularization), comprising the step of administering to a patient a therapeutically effective amount of a compound (e.g., fusion proteins of the invention and/or polynucleotides encoding albumin fusion proteins of the invention) to the cornea, such that the formation of blood vessels is inhibited. Briefly, the cornea is a tissue which normally lacks blood vessels. In certain pathological conditions however, capillaries may extend into the cornea from the pericorneal vascular plexus of the limbus. When the cornea becomes vascularized, it also becomes clouded, resulting in a decline in the patient's visual acuity. Visual loss may become complete if the cornea completely opacitates. A wide variety of disorders can result in corneal neovascularization, including for example, corneal infections (e.g., trachoma, herpes simplex keratitis, leishmaniasis and onchocerciasis), immunological processes (e.g., graft rejection and Stevens-Johnson's syndrome), alkali burns, trauma, inflammation (of any cause), toxic and nutritional deficiency states, and as a complication of wearing contact lenses.

Within yet further embodiments of the invention, may be prepared for topical administration in saline (combined with any of the preservatives and antimicrobial agents commonly used in ocular preparations), and administered in eyedrop form. The solution or suspension may be prepared in its pure form and administered several times daily. Alternatively, anti-angiogenic compositions, prepared as described above, may also be administered directly to the cornea. Within other embodiments, the anti-angiogenic composition is prepared with a muco-adhesive polymer which binds to cornea. Within further embodiments, the anti-angiogenic factors or anti-angiogenic compositions may be utilized as an adjunct to conventional steroid therapy. Topical therapy may also be useful prophylactically in corneal lesions which are known to have a high probability of inducing an angiogenic response (such as chemical burns). In these instances the treatment, likely in combination with steroids, may be instituted immediately to help prevent subsequent complications.

Within other embodiments, the compounds described above may be injected directly into the corneal stroma by an ophthalmologist under microscopic guidance. The site of injection may vary with the morphology of the individual lesion, but the goal of the administration would be to place the composition at the advancing front of the vasculature (i.e., interspersed between the blood vessels and the normal cornea). In most cases this would involve perilimbic corneal injection to “protect” the cornea from the advancing blood vessels. This method may also be utilized shortly after a corneal insult in order to prophylactically prevent corneal neovascularization. In this situation the material could be injected in the perilimbic cornea interspersed between the corneal lesion and its undesired potential limbic blood supply. Such methods may also be utilized in a similar fashion to prevent capillary invasion of transplanted corneas. In a sustained-release form injections might only be required 2-3 times per year. A steroid could also be added to the injection solution to reduce inflammation resulting from the injection itself.

Within another aspect of the present invention, methods are provided for treating neovascular glaucoma, comprising the step of administering to a patient a therapeutically effective amount of an albumin fusion protein of the invention and/or polynucleotides encoding an albumin fusion protein of the invention to the eye, such that the formation of blood vessels is inhibited. In one embodiment, the compound may be administered topically to the eye in order to treat early forms of neovascular glaucoma. Within other embodiments, the compound may be implanted by injection into the region of the anterior chamber angle. Within other embodiments, the compound may also be placed in any location such that the compound is continuously released into the aqueous humor. Within another aspect of the present invention, methods are provided for treating proliferative diabetic retinopathy, comprising the step of administering to a patient a therapeutically effective amount of an albumin fusion protein of the invention and/or polynucleotides encoding an albumin fusion protein of the invention to the eyes, such that the formation of blood vessels is inhibited.

Within yet further embodiments of the invention, proliferative diabetic retinopathy may be treated by injection into the aqueous humor or the vitreous, in order to increase the local concentration of the polynucleotide, polypeptide, antagonist and/or agonist in the retina. This treatment could be initiated prior to the acquisition of severe disease requiring photocoagulation.

Within another aspect of the present invention, methods are provided for treating retrolental fibroplasia, comprising the step of administering to a patient a therapeutically effective amount of an albumin fusion protein of the invention and/or polynucleotides encoding an albumin fusion protein of the invention to the eye, such that the formation of blood vessels is inhibited. The compound may be administered topically, via intravitreous injection and/or via intraocular implants.

Additionally, disorders which can be treated with fusion proteins of the invention and/or polynucleotides encoding albumin fusion proteins of the invention include, but are not limited to, hemangioma, arthritis, psoriasis, angiofibroma, atherosclerotic plaques, delayed wound healing, granulations, hemophilic joints, hypertrophic scars, nonunion fractures, Osler-Weber syndrome, pyogenic granuloma, scleroderma, trachoma, and vascular adhesions.

Moreover, disorders and/or states, which can be treated, prevented, diagnosed, and/or prognosed with the the albumin fusion proteins of the invention and/or polynucleotides encoding albumin fusion proteins of the invention of the invention include, but are not limited to, solid tumors, blood born tumors such as leukemias, tumor metastasis, Kaposi's sarcoma, benign tumors, for example hemangiomas, acoustic neuromas, neurofibromas, trachomas, and pyogenic granulomas, rheumatoid arthritis, psoriasis, ocular angiogenic diseases, for example, diabetic retinopathy, retinopathy of prematurity, macular degeneration, corneal graft rejection, neovascular glaucoma, retrolental fibroplasia, rubeosis, retinoblastoma, and uvietis, delayed wound healing, endometriosis, vascluogenesis, granulations, hypertrophic scars (keloids), nonunion fractures, scleroderma, trachoma, vascular adhesions, myocardial angiogenesis, coronary collaterals, cerebral collaterals, arteriovenous malformations, ischemic limb angiogenesis, Osler-Webber Syndrome, plaque neovascularization, telangiectasia, hemophiliac joints, angiofibroma fibromuscular dysplasia, wound granulation, Crohn's disease, atherosclerosis, birth control agent by preventing vascularization required for embryo implantation controlling menstruation, diseases that have angiogenesis as a pathologic consequence such as cat scratch disease (Rochele minalia quintosa), ulcers (Helicobacter pylori), Bartonellosis and bacillary angiomatosis.

In one aspect of the birth control method, an amount of the compound sufficient to block embryo implantation is administered before or after intercourse and fertilization have occurred, thus providing an effective method of birth control, possibly a “morning after” method. Albumin fusion proteins of the invention and/or polynucleotides encoding albumin fusion proteins of the invention may also be used in controlling menstruation or administered as either a peritoneal lavage fluid or for peritoneal implantation in the treatment of endometriosis.

Albumin fusion proteins of the invention and/or polynucleotides encoding albumin fusion proteins of the invention may be incorporated into surgical sutures in order to prevent stitch granulomas.

Albumin fusion proteins of the invention and/or polynucleotides encoding albumin fusion proteins of the invention may be utilized in a wide variety of surgical procedures. For example, within one aspect of the present invention a compositions (in the form of, for example, a spray or film) may be utilized to coat or spray an area prior to removal of a tumor, in order to isolate normal surrounding tissues from malignant tissue, and/or to prevent the spread of disease to surrounding tissues. Within other aspects of the present invention, compositions (e.g., in the form of a spray) may be delivered via endoscopic procedures in order to coat tumors, or inhibit angiogenesis in a desired locale. Within yet other aspects of the present invention, surgical meshes which have been coated with anti-angiogenic compositions of the present invention may be utilized in any procedure wherein a surgical mesh might be utilized. For example, within one embodiment of the invention a surgical mesh laden with an anti-angiogenic composition may be utilized during abdominal cancer resection surgery (e.g., subsequent to colon resection) in order to provide support to the structure, and to release an amount of the anti-angiogenic factor.

Within further aspects of the present invention, methods are provided for treating tumor excision sites, comprising administering albumin fusion proteins of the invention and/or polynucleotides encoding albumin fusion proteins of the invention to the resection margins of a tumor subsequent to excision, such that the local recurrence of cancer and the formation of new blood vessels at the site is inhibited. Within one embodiment of the invention, the anti-angiogenic compound is administered directly to the tumor excision site (e.g., applied by swabbing, brushing or otherwise coating the resection margins of the tumor with the anti-angiogenic compound). Alternatively, the anti-angiogenic compounds may be incorporated into known surgical pastes prior to administration. Within particular embodiments of the invention, the anti-angiogenic compounds are applied after hepatic resections for malignancy, and after neurosurgical operations.

Within one aspect of the present invention, fusion proteins of the invention and/or polynucleotides encoding albumin fusion proteins of the invention may be administered to the resection margin of a wide variety of tumors, including for example, breast, colon, brain and hepatic tumors. For example, within one embodiment of the invention, anti-angiogenic compounds may be administered to the site of a neurological tumor subsequent to excision, such that the formation of new blood vessels at the site are inhibited.

The albumin fusion proteins of the invention and/or polynucleotides encoding albumin fusion proteins of the invention may also be administered along with other anti-angiogenic factors, such as those described in U.S. Provisional Application Ser. No. 60/355,547 and WO 01/79480.

Expression of Fusion Proteins

The albumin fusion proteins of the invention may be produced as recombinant molecules by secretion from yeast, a microorganism such as a bacterium, or a human or animal cell line. Optionally, the polypeptide is secreted from the host cells.

For expression of the albumin fusion proteins exemplified herein, yeast strains disrupted of the HSP150 gene as exemplified in WO 95/33833, or yeast strains disrupted of the PMT1 gene as exemplified in WO 00/44772 [rHA process] (serving to reduce/eliminate O-linked glycosylation of the albumin fusions), or yeast strains disrupted of the YAP3 gene as exemplified in WO 95/23857 were successfully used, in combination with the yeast PRB1 promoter, the HSA/MFα-1 fusion leader sequence exemplified in WO 90/01063, the yeast ADH1 terminator, the LEU2 selection marker and the disintegration vector pSAC35 exemplied in U.S. Pat. No. 5,637,504.

Other yeast strains, promoters, leader sequences, terminators, markers and vectors which are expected to be useful in the invention are described in U.S. Provisional Application Ser. No. 60/355,547 and in WO 01/74980 (pp. 94-99), which are incorporated herein by reference, and are well known in the art.

The present invention also includes a cell, optionally a yeast cell transformed to express an albumin fusion protein of the invention. In addition to the transformed host cells themselves, the present invention also contemplates a culture of those cells, optionally a monoclonal (clonally homogeneous) culture, or a culture derived from a monoclonal culture, in a nutrient medium. If the polypeptide is secreted, the medium will contain the polypeptide, with the cells, or without the cells if they have been filtered or centrifuged away. Many expression systems are known and may be used, including bacteria (for example E. coli and Bacillus subtilis), yeasts (for example Saccharomyces cerevisiae, Kluyveromyces lactis and Pichia pastoris), filamentous fungi (for example Aspergillus), plant cells, animal cells and insect cells.

The desired protein is produced in conventional ways, for example from a coding sequence inserted in the host chromosome or on a free plasmid. The yeasts are transformed with a coding sequence for the desired protein in any of the usual ways, for example electroporation. Methods for transformation of yeast by electroporation are disclosed in Becker & Guarente (1990) Methods Enzymol. 194, 182.

Successfully transformed cells, i.e., cells that contain a DNA construct of the present invention, can be identified by well known techniques. For example, cells resulting from the introduction of an expression construct can be grown to produce the desired polypeptide. Cells can be harvested and lysed and their DNA content examined for the presence of the DNA using a method such as that described by Southern (1975) J. Mol. Biol. 98, 503 or Berent et al. (1985) Biotech. 3, 208. Alternatively, the presence of the protein in the supernatant can be detected using antibodies.

Useful yeast plasmid vectors include pRS403-406 and pRS413-416 and are generally available from Stratagene Cloning Systems, La Jolla, Calif. 92037, USA. Plasmids pRS403, pRS404, pRS405 and pRS406 are Yeast Integrating plasmids (YIps) and incorporate the yeast selectable markers HIS3, TRP1, LEU2 and URA3. Plasmids pRS413-416 are Yeast Centromere plasmids (YCps).

Vectors for making albumin fusion proteins for expression in yeast include pPPC0005, pScCHSA, pScNHSA, and pC4:HSA which were deposited on Apr. 11, 2001 at the American Type Culture Collection, 10801 University Boulevard, Manassas, Va. 20110-2209 and which are described in Provisional Application Ser. No. 60/355,547 and WO 01/79480, which are incorporated by reference herein.

Another vector which is expected to be useful for expressing an albumin fusion protein in yeast is the pSAC35 vector which is described in Sleep et al., BioTechnology 8:42 (1990), which is hereby incorporated by reference in its entirety. The plasmid pSAC35 is of the disintegration class of vector described in U.S. Pat. No. 5,637,504.

A variety of methods have been developed to operably link DNA to vectors via complementary cohesive termini. For instance, complementary homopolymer tracts can be added to the DNA segment to be inserted to the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, generated by endonuclease restriction digestion, is treated with bacteriophage T4 DNA polymerase or E. coli DNA polymerase I, enzymes that remove protruding, γ-single-stranded termini with their 3′5′-exonucleolytic activities, and fill in recessed 3′-ends with their polymerizing activities. The combination of these activities therefore generates blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces termini compatible with those of the DNA segment.

Synthetic linkers containing a variety of restriction endonuclease sites are commercially available from a number of commercial sources.

A desirable way to modify the DNA in accordance with the invention, if, for example, HA variants are to be prepared, is to use the polymerase chain reaction as disclosed by Saiki et al (1988) Science 239, 487-491. In this method the DNA to be enzymatically amplified is flanked by two specific oligonucleotide primers which themselves become incorporated into the amplified DNA. The specific primers may contain restriction endonuclease recognition sites which can be used for cloning into expression vectors using methods known in the art.

Exemplary genera of yeast contemplated to be useful in the practice of the present invention as hosts for expressing the albumin fusion proteins are Pichia (formerly classified as Hansenula), Saccharomyces, Kluyveronzyces, Aspergillus, Candida, Torulopsis, Torulaspora, Schizosaccharomyces, Citeromyces, Pachysolen, Zygosaccharomyces, Debaromyces, Trichoderma, Cephalosporium, Humicola, Mucor, Neurospora, Yarrowia, Metschunikowia, Rhodosporidium, Leucosporidium, Botryoascus, Sporidiobolus, Endomycopsis, and the like. Genera include those selected from the group consisting of Saccharomyces, Schizosaccharomyces, Kluyveromyces, Pichia and Torulaspora. Examples of Saccharomyces spp. are S. cerevisiae, S. italicus and S. rouxii. Examples of other species, and methods of transforming them, are described in U.S. Provisional Application Ser. No. 60/355,547 and WO 01/79480 (pp. 97-98), which are incorporated herein by reference.

Methods for the transformation of S. cerevisiae are taught generally in EP 251 744, EP 258 067 and WO 90/01063, all of which are incorporated herein by reference.

Suitable promoters for S. cerevisiae include those associated with the PGKI gene, GAL1 or GAL10 genes, CYCI, PHO5, TRPI, ADHI, ADH2, the genes for glyceraldehyde-3-phosphate dehydrogenase, hexokinase, pyruvate decarboxylase, phosphofructokinase, triose phosphate isomerase, phosphoglucose isomerase, glucokinase, alpha-mating factor pheromone, [a mating factor pheromone], the PRBI promoter, the GUT2 promoter, the GPDI promoter, and hybrid promoters involving hybrids of parts of 5′ regulatory regions with parts of 5′ regulatory regions of other promoters or with upstream activation sites (e.g. the promoter of EP-A-258 067).

Convenient regulatable promoters for use in Schizosaccharomyces pombe are the thiamine-repressible promoter from the nmt gene as described by Maundrell (1990) J. Biol. Chem. 265, 10857-10864 and the glucose repressible jbpl gene promoter as described by Hoffman & Winston (1990) Genetics 124, 807-816.

Methods of transforming Pichia for expression of foreign genes are taught in, for example, Cregg et al. (1993), and various Phillips patents (e.g. U.S. Pat. No. 4,857,467, incorporated herein by reference), and Pichia expression kits are commercially available from Invitrogen BV, Leek, Netherlands, and Invitrogen Corp., San Diego, Calif. Suitable promoters include AOXI and AOX2. Gleeson et al. (1986) J. Gen. Microbiol. 132, 3459-3465 include information on Hansenula vectors and transformation, suitable promoters being MOX1 and FMD1; whilst EP 361 991, Fleer et al. (1991) and other-publications from Rhone-Poulenc Rorer teach how to express foreign proteins in Kluyveromyces spp.

The transcription termination signal may be the 3′ flanking sequence of a eukaryotic gene which contains proper signals for transcription termination and polyadenylation. Suitable 3′ flanking sequences may, for example, be those of the gene naturally linked to the expression control sequence used, i.e. may correspond to the promoter. Alternatively, they may be different in which case the termination signal of the S. cerevisiae ADHI gene is optionally used.

The desired albumin fusion protein may be initially expressed with a secretion leader sequence, which may be any leader effective in the yeast chosen. Leaders useful in S. cerevisiae include that from the mating factor α polypeptide (MF α-1) and the hybrid leaders of EP-A-387 319. Such leaders (or signals) are cleaved by the yeast before the mature albumin is released into the surrounding medium. Further such leaders include those of S. cerevisiae invertase (SUC2) disclosed in JP 62-096086 (granted as 911036516), acid phosphatase (PH05), the pre-sequence of MFα-1, 0 glucanase (BGL2) and killer toxin; S. diastaticus glucoamylase II; S. carlsbergensis α-galactosidase (MEL1); K lactis killer toxin; and Candida glucoamylase.

Additional Methods of Recombinant and Synthetic Production of Albumin Fusion Proteins

The present invention includes polynucleotides encoding albumin fusion proteins of this invention, as well as vectors, host cells and organisms containing these polynucleotides. The present invention also includes methods of producing albumin fusion proteins of the invention by synthetic and recombinant techniques. The polynucleotides, vectors, host cells, and organisms may be isolated and purified by methods known in the art

A vector useful in the invention may be, for example, a phage, plasmid, cosmid, mini-chromosome, viral or retroviral vector.

The vectors which can be utilized to clone and/or express polynucleotides of the invention are vectors which are capable of replicating and/or expressing the polynucleotides in the host cell in which the polynucleotides are desired to be replicated and/or expressed. In general, the polynucleotides and/or vectors can be utilized in any cell, either eukaryotic or prokaryotic, including mammalian cells (e.g., human (e.g., HeLa), monkey (e.g., Cos), rabbit (e.g., rabbit reticulocytes), rat, hamster (e.g., CHO, NSO and baby hamster kidney cells) or mouse cells (e.g., L cells), plant cells, yeast cells, insect cells or bacterial cells (e.g., E. coli). See, e.g., F. Ausubel et al., Current Protocols in Molecular Biology, Greene Publishing Associates and Wiley-Interscience (1992) and Sambrook et al. (1989) for examples of appropriate vectors for various types of host cells. Note, however, that when a retroviral vector that is replication defective is ued, viral propagation generally will occur only in complementing host cells.

The host cells containing these polynucleotides can be used to express large amounts of the protein useful in, for example, pharmaceuticals, diagnostic reagents, vaccines and therapeutics. The protein may be isolated and purified by methods known in the art or described herein.

The polynucleotides encoding albumin fusion proteins of the invention may be joined to a vector containing a selectable marker for propagation in a host. Generally, a plasmid vector may be introduced in a precipitate, such as a calcium phosphate precipitate, or in a complex with a charged lipid. If the vector is a virus, it may be packaged in vitro using an appropriate packaging cell line and then transduced into host cells.

The polynucleotide insert should be operatively linked to an appropriate promoter compatible with the host cell in which the polynucleotide is to be expressed. The promoter may be a strong promoter and/or an inducible promoter. Examples of promoters include the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs may include a translation initiating codon at the beginning and a termination codon (UAA, UGA or UAG) appropriately positioned at the end of the polypeptide to be translated.

As indicated, the expression vectors may include at least one selectable marker. Such markers include dihydrofolate reductase, G418, glutamine synthase, or neomycin resistance for eukaryotic cell culture, and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, NSO, 293, and Bowes melanoma cells; and plant cells. Appropriate culture mediums and conditions for the above-described host cells are known in the art.

In one embodiment, polynucleotides encoding an albumin fusion protein of the invention may be fused to signal sequences which will direct the localization of a protein of the invention to particular compartments of a prokaryotic or eukaryotic cell and/or direct the secretion of a protein of the invention from a prokaryotic or eukaryotic cell. For example, in E. coli, one may wish to direct the expression of the protein to the periplasmic space. Examples of signal sequences or proteins (or fragments thereof) to which the albumin fusion proteins of the invention may be fused in order to direct the expression of the polypeptide to the periplasmic space of bacteria include, but are not limited to, the pelB signal sequence, the maltose binding protein (MBP) signal sequence, MBP, the ompA signal sequence, the signal sequence of the periplasmic E. coli heat-labile enterotoxin B-subunit, and the signal sequence of alkaline phosphatase. Several vectors are commercially available for the construction of fusion proteins which will direct the localization of a protein, such as the pMAL series of vectors (particularly the pMAL-p series) available from New England Biolabs. In a specific embodiment, polynucleotides albumin fusion proteins of the invention may be fused to the pelB pectate lyase signal sequence to increase the efficiency of expression and purification of such polypeptides in Gram-negative bacteria. See, U.S. Pat. Nos. 5,576,195 and 5,846,818, the contents of which are herein incorporated by reference in their entireties.

Examples of signal peptides that may be fused to an albumin fusion protein of the invention in order to direct its secretion in mammalian cells include, but are not limited to, the MPIF-1 signal sequence (e.g., amino acids 1-21 of GenBank Accession number AAB51134), the stanniocalcin signal sequence (MLQNSAVLLLLVISASA, SEQ ID NO:5), and a consensus signal sequence (MPTWAWWLFLVLLLALWAPARG, SEQ ID NO:6). A suitable signal sequence that may be used in conjunction with baculoviral expression systems is the gp67 signal sequence (e.g., amino acids 1-19 of GenBank Accession Number AAA72759).

Vectors which use glutamine synthase (GS) or DHFR as the selectable markers can be amplified in the presence of the drugs methionine sulphoximine or methotrexate, respectively. An advantage of glutamine synthase based vectors are the availabilty of cell lines (e.g., the murine myeloma cell line, NSO) which are glutamine synthase negative. Glutamine synthase expression systems can also function in glutamine synthase expressing cells (e.g., Chinese Hamster Ovary (CHO) cells) by providing additional inhibitor to prevent the functioning of the endogenous gene. A glutamine synthase expression system and components thereof are detailed in PCT publications: WO87/04462; WO86/05807; WO89/01036; WO89/10404; and WO91/06657, which are hereby incorporated in their entireties by reference herein. Additionally, glutamine synthase expression vectors can be obtained from Lonza Biologics, Inc. (Portsmouth, N.H.). Expression and production of monoclonal antibodies using a GS expression system in murine myeloma cells is described in Bebbington et al., Bio/technology 10:169(1992) and in Biblia and Robinson Biotechnol. Prog. 11:1 (1995) which are herein incorporated by reference.

The present invention also relates to host cells containing vector constructs, such as those described herein, and additionally encompasses host cells containing nucleotide sequences of the invention that are operably associated with one or more heterologous control regions (e.g., promoter and/or enhancer) using techniques known of in the art. The host cell can be a higher eukaryotic cell, such as a mammalian cell (e.g., a human derived cell), or a lower eukaryotic cell, such as a yeast cell, or the host cell can be a prokaryotic cell, such as a bacterial cell. A host strain may be chosen which modulates the expression of the inserted gene sequences, or modifies and processes the gene product in the specific fashion desired. Expression from certain promoters can be elevated in the presence of certain inducers; thus expression of the genetically engineered polypeptide may be controlled. Furthermore, different host cells have characteristics and specific mechanisms for the translational and post-translational processing and modification (e.g., phosphorylation, cleavage) of proteins. Appropriate cell lines can be chosen to ensure the desired modifications and processing of the foreign protein expressed.

Introduction of the nucleic acids and nucleic acid constructs of the invention into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that the polypeptides of the present invention may in fact be expressed by a host cell lacking a recombinant vector.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g., the coding sequence corresponding to a Therapeutic protein may be replaced with an albumin fusion protein corresponding to the Therapeutic protein), and/or to include genetic material (e.g., heterologous polynucleotide sequences such as for example, an albumin fusion protein of the invention corresponding to the Therapeutic protein may be included). The genetic material operably associated with the endogenous polynucleotide may activate, alter, and/or amplify endogenous polynucleotides.

In addition, techniques known in the art may be used to operably associate heterologous polynucleotides (e.g., polynucleotides encoding an albumin protein, or a fragment or variant thereof) and/or heterologous control regions (e.g., promoter and/or enhancer) with endogenous polynucleotide sequences encoding a Therapeutic protein via homologous recombination (see, e.g., U.S. Pat. No. 5,641,670, issued Jun. 24, 1997; International Publication Number WO 96/29411; International Publication Number WO 94/12650; Koller et al., Proc. Natl. Acad. Sci. USA 86:8932-8935 (1989); and Zijlstra et al, Nature 342:435-438 (1989), the disclosures of each of which are incorporated by reference in their entireties).

Advantageously, albumin fusion proteins of the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, hydrophobic charge interaction chromatography and lectin chromatography. In some embodiments, high performance liquid chromatography (“HPLC”) may be employed for purification.

In some embodiments albumin fusion proteins of the invention are purified using one or more Chromatography methods listed above. In other embodiments, albumin fusion proteins of the invention are purified using one or more of the following Chromatography columns, Q sepharose FF column, SP Sepharose FF column, Q Sepharose High Performance Column, Blue Sepharose FF column, Blue Column, Phenyl Sepharose FF column, DEAE Sepharose FF, or Methyl Column.

Additionally, albumin fusion proteins of the invention may be purified using the process described in International Publication No. WO 00/44772 which is herein incorporated by reference in its entirety. One of skill in the art could easily modify the process described therein for use in the purification of albumin fusion proteins of the invention.

Albumin fusion proteins of the present invention may be recovered from products produced by recombinant techniques from a prokaryotic or eukaryotic host, including, for example, bacterial, yeast, higher plant, insect, and mammalian cells. Depending upon the host employed in a recombinant production procedure, the polypeptides of the present invention may be glycosylated or may be non-glycosylated. In addition, albumin fusion proteins of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

Albumin fusion proteins of the invention and antibodies that bind a Therapeutic protein or fragments or variants thereof can be fused to marker sequences, such as a peptide to facilitate purification. In one embodiment, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “FLAG” tag.

Further, an albumin fusion protein of the invention may be conjugated to a therapeutic moiety such as a cytotoxin, e.g., a cytostatic or cytocidal agent, a therapeutic agent or a radioactive metal ion, e.g., alpha-emitters such as, for example, 213Bi. Examples of such agents are given in U.S. Provisional Application Ser. No. 60/355,547 and in WO 01/79480 (p. 107), which are incorporated herein by reference.

Albumin fusion proteins may also be attached to solid supports, which are particularly useful for immunoassays or purification of polypeptides that are bound by, that bind to, or associate with albumin fusion proteins of the invention. Such solid supports include, but are not limited to, glass, cellulose, polyacrylamide, nylon, polystyrene, polyvinyl chloride or polypropylene.

Also provided by the invention are chemically modified derivatives of the albumin fusion proteins of the invention which may provide additional advantages such as increased solubility, stability and circulating time of the polypeptide, or decreased immunogenicity (see U.S. Pat. No. 4,179,337). Examples involving the use of polyethylene glycol are given in WO 01/79480 (pp. 109-111), which are incorporated by reference herein.

The presence and quantity of albumin fusion proteins of the invention may be determined using ELISA, a well known immunoassay known in the art.

Uses of the Polypeptides

Each of the polypeptides identified herein can be used in numerous ways. The following description should be considered exemplary and utilizes known techniques.

The albumin fusion proteins of the present invention are useful for treatment, prevention and/or prognosis of various disorders in mammals, preferably humans. Such disorders include, but are not limited to, those described herein under the heading “Biological Activity” in Table 1.

The albumin fusion proteins of the invention may be used as inhibitors of proliferation of endothelial cells and tumor-induced angiogenesis.

Moreover, albumin fusion proteins of the present invention can be used to treat or prevent diseases or conditions. For example, the albumin fusion proteins of the invention may be used as a prophylactic or therapeutic for preventing growth of, or promoting regression of, primary tumors and metastases; and for treating cancer, diabetic retinopathy, progressive macular degeneration or theumatoid arthritis.

Albumin fusion proteins can be used to assay levels of polypeptides in a biological sample, such as in in vivo diagnostics. For example, radiolabeled albumin fusion proteins of the invention could be used for imaging of polypeptides in a body. Examples of assays are given, e.g., in U.S. Provisional Application Ser. No. 60/355,547 and WO 0179480 (pp. 112-122), which are incorporated herein by reference, and are well known in the art. Labels or markers for in vivo imaging of protein include, but are not limited to, those detectable by X-radiography, nuclear magnetic resonance (NMR), electron spin relaxtion (ESR), positron emission tomography (PET), or computer tomography (CT). For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the albumin fusion protein by labeling of nutrients given to a cell line expressing the albumin fusion protein of the invention.

An albumin fusion protein which has been labeled with an appropriate detectable imaging moiety, such as a radioisotope (for example, ¹³¹I, ¹¹²In, ^{99m}Tc, (¹³¹I, ¹²⁵I, ¹²³I, ¹²¹I), carbon (¹⁴C), sulfur (³⁵S), tritium (³H), indium (^{115m}In, ^{113m}In, ¹¹²In), and technetium (⁹⁹Tc, ^{99m}Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F, ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru), a radio-opaque substance, or a material detectable by nuclear magnetic resonance, is introduced (for example, parenterally, subcutaneously or intraperitoneally) into the mammal to be examined for immune system disorder. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ^{99m}Tc. The labeled albumin fusion protein will then preferentially accumulate at locations in the body (e.g., organs, cells, extracellular spaces or matrices) where one or more receptors, ligands or substrates (corresponding to that of the Therapeutic protein used to make the albumin fusion protein of the invention) are located. Alternatively, in the case where the albumin fusion protein comprises at least a fragment or variant of a Therapeutic antibody, the labeled albumin fusion protein will then preferentially accumulate at the locations in the body (e.g., organs, cells, extracellular spaces or matrices) where the polypeptides/epitopes corresponding to those bound by the Therapeutic antibody (used to male the albumin fusion protein of the invention) are located. In vivo tumor imaging is described in S. W. Burchiel et al., “Immunopharmacokinetics of Radiolabeled Antibodies and Their Fragments” (Chapter 13 in Tumor Imaging: The Radiochemical Detection of Cancer, S. W. Burchiel and B. A. Rhodes, eds., Masson Publishing Inc. (1982)). The protocols described therein could easily be modified by one of skill in the art for use with the albumin fusion proteins of the invention.

Thus, one aspect of the invention is the provision of a methods of diagnosing an anti-angiogenesis related disease or disorder in a mammal comprising administering a labeled fusion protein of the invention to a mammal, allowing at least some of the labeled fusion protein to reach the site of the angiogenesis dependent disease or disorder, and detecting the fusion protein at the site of the angiogenesis dependent disease or disorder. Such methods may be used, for example, to determine whehter an angiogenesis related disease or disorder has responded to treatment. Such methods may involve, for example, first determining the number or size of tumor(s) in a mammal and then determining whether the number of tumors has increased or decreased after treatment and/or or determining whether or not the tumor(s) has grown or mobilized after treatment.

Albumin fusion proteins of the invention can also be used to raise antibodies, which in turn may be used to measure protein expression of the Therapeutic protein, albumin protein, and/or the albumin fusion protein of the invention from a recombinant cell, as a way of assessing transformation of the host cell, or in a biological sample. Moreover, the albumin fusion proteins of the present invention can be used to test the biological activities described herein.

Transgenic Organisms

Transgenic organisms that express the albumin fusion proteins of the invention are also included in the invention. Transgenic organisms are genetically modified organisms into which recombinant, exogenous or cloned genetic material has been transferred. Such genetic material is often referred to as a transgene. The nucleic acid sequence of the transgene may include one or more transcriptional regulatory sequences and other nucleic acid sequences such as introns, that may be necessary for optimal expression and secretion of the encoded protein. The transgene may be designed to direct the expression of the encoded protein in a manner that facilitates its recovery from the organism or from a product produced by the organism, e.g. from the milk, blood, urine, eggs, hair or seeds of the organism. The transgene may consist of nucleic acid sequences derived from the genome of the same species or of a different species than the species of the target animal. The transgene may be integrated either at a locus of a genome where that particular nucleic acid sequence is not otherwise normally found or at the normal locus for the transgene.

The term “germ cell line transgenic organism” refers to a transgenic organism in which the genetic alteration or genetic information was introduced into a germ line cell, thereby conferring the ability of the transgenic organism to transfer the genetic information to offspring. If such offspring in fact possess some or all of that alteration or genetic information, then they too are transgenic organisms. The alteration or genetic information may be foreign to the species of organism to which the recipient belongs, foreign only to the particular individual recipient, or may be genetic information already possessed by the recipient. In the last case, the altered or introduced gene may be expressed differently than the native gene.

A transgenic organism may be a transgenic human, animal or plant. Transgenics can be produced by a variety of different methods including transfection, electroporation, microinjection, gene targeting in embryonic stem cells and recombinant viral and retroviral infection (see, e.g., U.S. Pat. No. 4,736,866; U.S. Pat. No. 5,602,307; Mullins et al. (1993) Hypertension 22(4):630-633; Brenin et al. (1997) Surg. Oncol. 6(2)99-110; Tuan (ed.), Recombinant Gene Expression Protocols, Methods in Molecular Biology No. 62, Humana Press (1997)). The method of introduction of nucleic acid fragments into recombination competent mammalian cells can be by any method which favors co-transformation of multiple nucleic acid molecules. Detailed procedures for producing transgenic animals are readily available to one skilled in the art, including the disclosures in U.S. Pat. No. 5,489,743 and U.S. Pat. No. 5,602,307. Additional information is given in U.S. Provisional Application Ser. No. 60/355,547 and WO 01/79480 (pp. 151-162), which are incorporated by reference herein.

Gene Therapy

Constructs encoding albumin fusion proteins of the invention can be used as a part of a gene therapy protocol to deliver therapeutically effective doses of the albumin fusion protein. One approach for in vivo introduction of nucleic acid into a cell is by use of a viral vector containing nucleic acid, encoding an albumin fusion protein of the invention. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid. The extended plasma half-life of the described albumin fusion proteins might even compensate for a potentially low expression level.

Retrovirus vectors and adeno-associated virus vectors can be used as a recombinant gene delivery system for the transfer of exogenous nucleic acid molecules encoding albumin fusion proteins in vivo. These vectors provide efficient delivery of nucleic acids into cells, and the transferred nucleic acids are stably integrated into the chromosomal DNA of the host. Examples of such vectors, methods of using them, and their advantages, as well as non-viral delivery methods are described in detail in U.S. Provisional Application Ser. No. 60/355,547 and WO 01/79480 (pp. 151-153), which are incorporated by reference herein.

Gene delivery systems for a gene encoding an albumin fusion protein of the invention can be introduced into a patient by any of a number of methods. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by Stereotactic injection (e.g. Chen et al. (1994) PNAS 91: 3054-3057). The pharmaceutical preparation of the gene therapy construct can consist essentially of the gene delivery system in an acceptable diluent, or can comprise a slow release matrix in which the gene delivery vehicle is imbedded. Where the albumin fusion protein can be produced intact from recombinant cells, e.g. retroviral vectors, the pharmaceutical preparation can comprise one or more cells which produce the albumin fusion protein. Additional gene therapy methods are described in U.S. Provisional Application Ser. No. 60/355,547 and in WO 01/79480 (pp. 153-162), which are incorporated herein by reference.

Pharmaceutical or Therapeutic Compositions

The albumin fusion proteins of the invention or formulations thereof may be administered by any conventional method including parenteral (e.g. subcutaneous or intramuscular) injection or intravenous infusion. The treatment may consist of a single dose or a plurality of doses over a period of time. Furthermore, the dose, or plurality of doses, is administered less frequently than for the Therapeutic Protein which is not fused to albumin.

While it is possible for an albumin fusion protein of the invention to be administered alone, it is desirable to present it as a pharmaceutical formulation, together with one or more acceptable carriers. The carrier(s) must be “acceptable” in the sense of being compatible with the albumin fusion protein and not deleterious to the recipients thereof. Typically, the carriers will be water or saline which will be sterile and pyrogen free. Albumin fusion proteins of the invention are particularly well suited to formulation in aqueous carriers such as sterile pyrogen free, water, saline or other isotonic solutions because of their extended shelf-life in solution. For instance, pharmaceutical compositions of the invention may be formulated well in advance in aqueous form, for instance, weeks or months or longer time periods before being dispensed.

Formulations containing the albumin fusion protein may be prepared talking into account the extended shelf-life of the albumin fusion protein in aqueous formulations. As discussed above, the shelf-life of many of these Therapeutic proteins are markedly increased or prolonged after fusion to HA.

In instances where aerosol administration is appropriate, the albumin fusion proteins of the invention can be formulated as aerosols using standard procedures. The term “aerosol” includes any gas-borne suspended phase of an albumin fusion protein of the instant invention which is capable of being inhaled into the bronchioles or nasal passages. Specifically, aerosol includes a gas-borne suspension of droplets of an albumin fusion protein of the instant invention, as may be produced in a metered dose inhaler or nebulizer, or in a mist sprayer. Aerosol also includes a dry powder composition of a compound of the instant invention suspended in air or other carrier gas, which may be delivered by insufflation from an inhaler device, for example.

The formulations may conveniently be presented in unit dosage form and may be prepared by any of the methods well known in the art of pharmacy. Such methods include the step of bringing into association the albumin fusion protein with the carrier that constitutes one or more accessory ingredients. In general the formulations are prepared by uniformly and intimately bringing into association the active ingredient with liquid carriers or finely divided solid carriers or both, and then, if necessary, shaping the product.

Formulations suitable for parenteral administration include aqueous and non-aqueous sterile injection solutions which may contain anti-oxidants, buffers, bacteriostats and solutes which render the formulation appropriate for the intended recipient; and aqueous and non-aqueous sterile suspensions which may include suspending agents and thickening agents. The formulations may be presented in unit-dose or multi-dose containers, for example sealed ampules, vials or syringes, and may be stored in a freeze-dried (lyophilised) condition requiring only the addition of the sterile liquid carrier, for example water for injections, immediately prior to use. Extemporaneous injection solutions and suspensions may be prepared from sterile powders. Dosage formulations may contain the Therapeutic protein portion at a lower molar concentration or lower dosage compared to the non-fused standard formulation for the Therapeutic protein given the extended serum half-life exhibited by many of the albumin fusion proteins of the invention.

As an example, when an albumin fusion protein of the invention comprises one or more of the Therapeutic protein regions, the dosage form can be calculated on the basis of the potency of the albumin fusion protein relative to the potency of the Therapeutic protein, while taking into account the prolonged serum half-life and shelf-life of the albumin fusion proteins compared to that of the native Therapeutic protein. For example, in an albumin fusion protein consisting of a full length HA fused to a full length Therapeutic protein, an equivalent dose in terms of units would represent a greater weight of agent but the dosage frequency can be reduced.

Formulations or compositions of the invention may be packaged together with, or included in a kit with, instructions or a package insert referring to the extended shelf-life of the albumin fusion protein component. For instance, such instructions or package inserts may address recommended storage conditions, such as time, temperature and light, talking into account the extended or prolonged shelf-life of the albumin fusion proteins of the invention. Such instructions or package inserts may also address the particular advantages of the albumin fusion proteins of the inventions, such as the ease of storage for formulations that may require use in the field, outside of controlled hospital, clinic or office conditions. As described above, formulations of the invention may be in aqueous form and may be stored under less than ideal circumstances without significant loss of therapeutic activity.

The invention also provides methods of treatment and/or prevention of diseases or disorders (such as, for example, any one or more of the diseases or disorders disclosed herein) by administration to a subject of an effective amount of an albumin fusion protein of the invention or a polynucleotide encoding an albumin fusion protein of the invention (“albumin fusion polynucleotide”) in a pharmaceutically acceptable carrier.

Effective dosages of the albumin fusion protein and/or polynucleotide of the invention to be administered may be determined through procedures well known to those in the art which address such parameters as biological half-life, bioavailability, and toxicity, including using data from routine in vitro and in vivo studies such as those described in the references in Table 1, using methods well known to those skilled in the art.

The albumin fusion protein and/or polynucleotide will be formulated and dosed in a fashion consistent with good medical practice, talking into account the clinical condition of the individual patient (especially the side effects of treatment with the albumin fusion protein and/or polynucleotide alone), the site of delivery, the method of administration, the scheduling of administration, and other factors known to practitioners. The “effective amount” for purposes herein is thus determined by such considerations.

For example, determining an effective amount of substance to be delivered can depend upon a number of factors including, for example, the chemical structure and biological activity of the substance, the age and weight of the patient, the precise condition requiring treatment and its severity, and the route of administration. The frequency of treatments depends upon a number of factors, such as the amount of albumin fusion protein or polynucleotide constructs administered per dose, as well as the health and history of the subject. The precise amount, number of doses, and timing of doses will be determined by the attending physician or veterinarian.

Albumin fusion proteins and polynucleotides of the present invention can be administered to any animal, preferably to mammals and birds. Preferred mammals include humans, dogs, cats, mice, rats, rabbits sheep, cattle, horses and pigs, with humans being particularly preferred.

As a general proposition, the albumin fusion protein of the invention will be dosed lower (on the molar basis of the unfused Therapeutic protein) or administered less frequently than the unfused Therapeutic protein. A therapeutically effective dose may refer to that amount of the compound sufficient to result in amelioration of symptoms or disease stabilisation or a prolongation of survival in a patient or improvement of quality of life.

The albumin fusion proteins of the invention are advantageous in that they can simulate continous infusion of “classic drugs”, i.e., less protein equivalent is needed for identical inhibitory activity. Due to prolonged half-life, CT-Endo may be adminstered, for example, s.c. every 3 days, NT-Endo, for example, every 2 days.

The albumin fusion proteins of the invention have the following additional advantages: (i) dose optimization design on the basis of the angiogeneic phenotype of a tumor to fit specific growth characteristics of individual tumors (e.g. fast and slow growing); (ii) controlling/avoiding unwanted accumulation of drug in longer applications which could result in fewer or lessened side reactions or altered efficacy. Furthermore, when peptides are hydrophobic in nature, their fusion to albumin improves their solubility which should also result in an increase of bioavailability and should allow for higher concentrated formulations.

Albumin fusion proteins and/or polynucleotides can be are administered orally, rectally, parenterally, intracisternally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any. The term “parenteral” as used herein refers to modes of administration which include intravenous, intramuscular, intraperitoneal, intrasternal, subcutaneous and intraarticular injection and infusion.

Albumin fusion proteins and/or polynucleotides of the invention are also suitably administered by sustained-release systems such as those described, e.g., in U.S. Provisional Application Ser. No. 60/355,547 and WO 01/79480 (pp. 129-130), which are incorporated herein by reference.

For parenteral administration, in one embodiment, the albumin fusion protein and/or polynucleotide is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation optionally does not include-oxidizing agents and other compounds that are known to be deleterious to the Therapeutic.

The albumin fusion proteins and/or polynucleotides of the invention may be administered alone or in combination with other therapeutic agents. Albumin fusion protein and/or polynucleotide agents that may be administered in combination with the albumin fusion proteins and/or polynucleotides of the invention, include but not limited to, chemotherapeutic agents, antibiotics, steroidal and non-steroidal anti-inflammatories, conventional immunotherapeutic agents, and/or therapeutic treatments as described, e.g., in U.S. Provisional Application Ser. No. 60/355,547 and WO 01/79480 (pp. 132-151) which are incorporated by reference herein. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

Pharmaceutical compositions suitable for use in the present invention include compositions wherein the active ingredients are contained in an effective amount to achieve its intended purpose.

The invention also provides a pharmaceutical pack or kit comprising one or more containers filled with one or more of the ingredients of the pharmaceutical compositions comprising albumin fusion proteins of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

Having generally described the invention, the same will be more readily understood by reference to the following examples, which are provided by way of illustration and are not intended as limiting.

Without further description, it is believed that one of ordinary skill in the art can, using the preceding description and the following illustrative examples, make and utilize the alterations detected in the present invention and practice the claimed methods. The following working examples therefore, specifically point out certain embodiments of the present invention, and are not to be construed as limiting in any way the remainder of the disclosure.

### EXAMPLES

### Example 1

### Cloning of a Human Endostatin cDNA

DNA from a human foetal kidney 5′-STRETCH Plus cDNA Library (Clonetech) was extracted by phenol/chloroform extraction, ethanol precipitated and then digested with RNaseA to remove any RNA present in the DNA sample. The DNA was serially diluted from 100 ng to 10 pg (in 10 fold increments). PCR primers JH005 and JH018 were designed to clone a BamHI site into the 5′ end of endostatin, and a HindIII site into the 3′ end of endostatin. The DNA sequence of each primer were as follows:

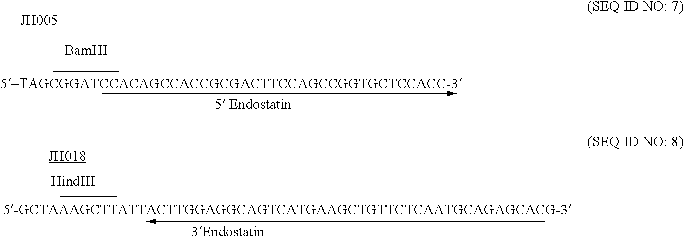

A master mix was prepared as follows: 2 mM MgCl₂ PCR Buffer, 10 μM PCR dNTP's, 0.2 μM JH005, 0.2 μM JH018, 2U FastStart Taq. DNA polymerase. 1 μL of template DNA (10 pg, 100 pg, 1 ng, 10 ng, 100 ng) was added to 49 μL of reaction mix. The total reaction volume was 50 μL. Perkin-Elmer Thermal Cycler 9600 was programmed as follows: denature at 95° C. for 4 mins [HOLD], then [CYLCE] denature at 95° C. for 30 s, anneal for 30 s at 45° C., extend at 72° C. for 60 s for 40 cycles, followed by a [HOLD] 72° C. for 600 s and then [HOLD] 4° C. The products of the PCR amplification were analysed by gel electrophoresis and a single DNA band of the expected size (0.57 kb) was observed. The modified endostatin cDNA fragment was isolated from the 1% (w/v) agarose TAE gel using a Gene Clean III Kit (BIO101 Inc.).

The endostatin cDNA fragment was digested to completion with BamHI/HindIII and ligated into BamHI/HindIII digested pBST+, described in WO 99/00504, to create plasmid pDB2446.

### Example 2

### Construction of C-Terminal and N-Terminal Albumin-Endostatin Expression Plasmids

Construction of C-Terminal Albumin-Endostatin Expression Plasmid

A C-terminal rHA-endostatin fusion were constructed wherein the C-terminal amino acid of albumin was followed by the first N-terminal amino acid of human endostatin.

A double stranded oligonucleotide linker was designed to manufacture the junction site between albumin and endostatin coding regions. The oligonucleotide pair JH012/JH013 was designed to extend from the Bsu36I site within albumin cDNA to the SexAI site within the 5′ region of endostatin cDNA. An AccI site was engineered into the 3′ end of the linker to allow the linker to be cloned into pDB2243, previously described in patent application WO 00/44772. Plasmid pDB2243, which contained the yeast PRB1 promoter and the yeast ADH1 terminator, provided appropriate transcription promoter and transcription terminator sequences.

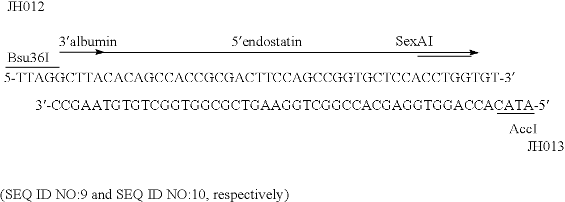

The oligonucleotide linker JH012/JH013 was ligated into the 6.13 kb Bsu36I-AccI fragment from pDB2243 to create plasmid pDB2442.

A synthetic self-complementary oligonucleotide JH011 was designed to insert a HindIII cloning site into a XhoI site of pDB2243, previously described in patent application WO 00/44772.

| | |
|---|---|
| JH011 | |
| HindIII | |
| 5′-TCGAGAAGCTTC-3′ | (SEQ ID NO:11) |

Plasmid pDB2243 was linearised at the unique XhoI just downstream of the yeast ADH1 transcription terminator. The oligonucleotide JH011 was annealed to itself to create a double stranded linker. The linker was ligated into XhoI linearised pDB2243 to create a plasmid pDB2441, which possessed a HindIII site, either side of the ADH1 terminator. Plasmid pDB2441 was digested to completion with HindIII and the 0.37 kb ADH1 terminator fragment was purified and ligated into HindIII digested pDB2446, which had been treated with calf intestinal phosphatase, to create plasmid pDB2450.

The next step in the construction of the albumin-endostatin fusion was dependant upon the use of the SexA1 restriction endonuclease. SexA1 is a Dcm-sensitive restriction enzyme. The dcm-, dam- E. coli strain GM2163 (New England Biolabs, genotype: F-, ara-14, leuB6, fhuA31, lacY1, tsx78, glnV44, galK2, galT22, mcrA, dcm-6, hisG4, rfbD1, rpsL136, dam13::Tn9, xylA5, mtl-1, rhi-1, mcrB1, hsdR2) was independently transformed with plasmids pDB2450 and pDB2442. Dcm- dam- pDB2450 and pDB2442 plasmid DNA was purified and digested to completion with BamHI and SexAI. The SexA1/BamHI fragment from pDB2450 (0.87 kb) was ligated into the SexA1/BamHI (5.88 kb) fragment from pDB2442 to create plasmid pDB2456.

Appropriate yeast vector sequences were provide by a “disintegration” plasmid pSAC35 generally disclosed in EP-A-286 424 and described by Sleep, D., et al. (1991) Bio/Technology 9, 183-187. The NotI C-terminal albumin-endostatin expression cassette was isolated from pDB2456, purified and ligated into NotI digested pSAC35 which had been treated with calf intestinal phosphatase, to create plasmid pDB2452 containing the NotI expression cassette in the same expression orientation as the LEU2 selectable marker.

Construction of N-Terminal Endostatin-Albumin Fusion Expression Plasmids

The recombinant albumin expression vectors pAYE645 and pAYE646 have been described previously in UK patent application 0217033.0. Plasmid pAYE645 contained the HSA/MFα-1 fusion leader sequence, as well as the yeast PRB1 promoter and the yeast ADH1 terminator providing appropriate transcription promoter and transcription terminator sequences is described in UK patent application 0217033.0. Plasmid pAYE645 was digested to completion with the restriction enzyme AflII and partially digested with the restriction enzyme HindIII and the DNA fragment comprising the 3′ end of the yeast PRB1 promoter and the albumin coding sequence was isolated. Plasmid pDB2241 described in patent application WO 00/44772, was digested with AflII/HindIII and the DNA fragment comprising the 5′ end of the yeast PRB1 promoter and the yeast ADH1 terminator was isolated. The AflII/HindIII DNA fragment from pAYE645 was then cloned into the AflII/HindIII pDB2241 vector DNA fragment to create the plasmid pDB2302. Plasmid pDB2302 was digested to completion with PacI/XhoI and the 6.19 kb fragment isolated, the recessed ends were blunt ended with T4 DNA polymerase and dNTPs, and religated to generate plasmid pDB2465. Plasmid pDB2465 was linearised with ClaI, the recessed ends were blunt ended with T4 DNA polymerase and dNTPs, and religated to generate plasmid pDB2533. Plasmid pDB2533 was linearised with BlnI, the recessed ends were blunt ended with T4 DNA polymerase and dNTPs, and religated to generate plasmid pDB2534. Plasmid pDB2534 was digested to completion with BmgBI/BglII, the 6.96 kb DNA fragment isolated and ligated to one of two double stranded oligonucleotide linkers, VC053/VC054 and VC057VC058 to create plasmid pDB2540, or VC055/VC056 and VC057/VC058 to create plasmid pDB2541.

| | |
|---|---|
| VC053 | |
| 5′-GATCTTTGGATAAGAGAGACGCTCACAAGTCCGAAGTCGCTCACCGGT-3′ | (SEQ ID NO:12) |
| VC054 | |
| 5′-pCCTTGAACCGGTGAGCGACTTCGGACTTGTGAGCGTCTCTCTTATCCAAA-3′ | (SEQ ID NO:13) |
| VC055 | |
| 5′-GATCTTTGGATAAGAGAGACGCTCACAAGTCCGAAGTCGCTCATCGAT-3′ | (SEQ ID NO:14) |
| VC056 | |
| 5′-pCCTTGAATCGATGAGCGACTTCGGACTTGTGAGCGTCTCTCTTATCCAAA-3′ | (SEQ ID NO:15) |
| VC057 | |
| 5′-pTCAAGGACCTAGGTGAGGAAAACTTCAAGGCTTTGGTCTTGATCGCTTTCG | (SEQ ID NO:16) |
| CTCAATACTTGCAACAATGTCCATTCGAAGATCAC-3′ | |
| VC058 | |
| 5′-GTGATCTTCGAATGGACATTGTTGCAAGTATTGAGCGAAAGCGATCAAGACC | (SEQ ID NO:19) |
| AAAGCCTTGAAGTTTTCCTCACCTAGGT-3′ | |

PCR primers JH029 and JH030 were designed to allow the endostatin cDNA to be cloned as an N-terminal albumin fusion into pDB2540 linearised with BglII and AgeI.

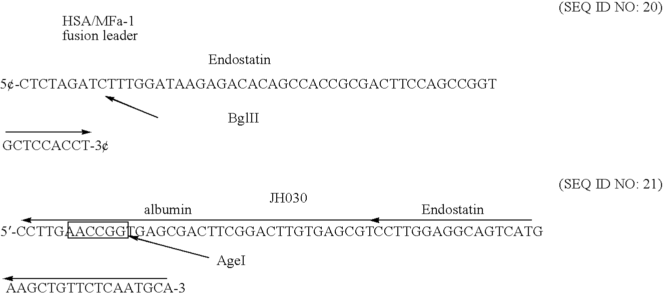

A master mix was prepared as follows: 2 mM MgCl₂ PCR Buffer, 10 μM PCR dNTP's, 0.2 μM JH029, 0.2 μM JH030, 2U FastStart Taq. DNA polymerase. 1 μL of pDB2446 (10 pg, 100 pg, 1 ng, 10 ng, 10 ng) was added to 49 μL of reaction mix. The total reaction volume was 50 μL. Perkin-Elmer Thermal Cycler 9600 was programmed as follows: Denature at 95° C. for 4 mins [HOLD], then [CYCLE] denature at 95° C. for 30 s, anneal for 30 s at 45° C., extend at 72° C. for 60 s for 20 cycles, followed by a [HOLD] 72° C. for 600 s and then [HOLD] 4° C. The products of the PCR amplification were analysed by gel electrophoresis and a band of expected size (0.59 kb) was observed. The 0.59 kb DNA fragment was isolated from the 1% (w/v) agarose TAE gel using Gene Clean III Kit (BIO101 Inc.).

The PCR DNA fragment was digested to completion with the restriction endonucleases BglII/AgeI and the 0.59 kb fragment was ligated into the 6.15 kb pDB2540 BglII/AgeI vector DNA fragment to create plasmid pDB2556.

Appropriate yeast vector sequences were provide by a “disintegration” plasmid pSAC35 generally disclosed in EP-A-286 424 and described by Sleep, D., et al. (1991) Bio/Technology 9, 183-187. The 3.54 kb NotI N-terminal endostatin-albumin expression cassette was isolated from pDB2556, purified and ligated into NotI digested pSAC35 which had been treated with calf intestinal phosphatase, creating plasmid pDB2557 contained the NotI expression cassette in the opposite orientation to the LEU2 selection marker.

### Example 3

### Cloning of a Human Angiostatin cDNA

A human liver 5′-STRETCH plus cDNA library (Clonetech) was selected as a source of a human angiostatin cDNA as the liver is the main producer of plasminogen. The DNA was extracted by phenol/chloroform extraction, ethanol precipitated and then digested with RNaseA to remove any RNA present in the DNA sample. The DNA was serially diluted from 100 ng to 10 pg (in 10 fold increments). Two mutagenic PCR primers JH003 and JH004 were designed to introduce a BamHI site into the 5′ end of angiostatin (JH004), and a HindIII site into the 3′ end of angiostatin (JH003).

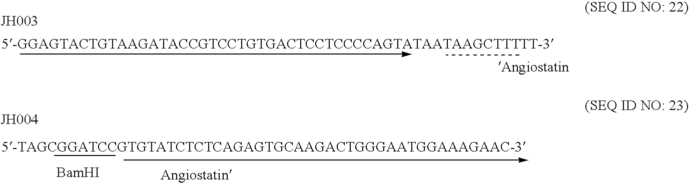

The angiostatin cDNA was amplified by PCR using the primers JH003 and JH004. A master mix was prepared as follows: 2 mM MgCl₂ PCR Buffer, 10 μM PCR dNTP's, 0.2 μM JH003, 0.2 μM JH004, 2U FastStart Taq. DNA polymerase (Roche). 1 μL of DNA (10 pg, 100 pg, 1 ng, 10 ng, 10 ng) was added to 49 μL of reaction mix. The total reaction volume was 50 μL. Perkin-Elmer Thermal Cycler 9600 was programmed as follows: denature at 95° C. for 4 mins [HOLD], then [CYLCE] denature at 95° C. for 30 s, anneal for 30 s at 45° C., extend at 72° C. for 60 s for 40 cycles, followed by a [HOLD] 72° C. for 600 s and then [HOLD] 4° C. The products of the PCR amplification were analysed by gel electrophoresis and a single DNA band of the expected size (0.79 kb) was observed. The modified angiostatin cDNA fragment was isolated from the 1% (w/v) agarose TAE gel using a Gene Clean III Kit (BIO101 Inc.). The angiostatin fragment was digested to completion with BamHI, HindIII (0.790 kb) and ligated into BamHI, HindIII digested pBST+, described in WO 99/00504, to generate plasmid pDB2447. The DNA sequence of the human angiostatin cDNA was obtained and aligned with the publicly available cDNA sequence from the National Centre For Biotechnology Information (NCBI) This analysis revealed that the DNA sequence had 100% identity with human plasminogen (RID: 998488083-23300-12247).

### Example 4

### Construction of C-Terminal and N-Terminal Albumin-Angiostatin Expression Plasmids

Construction of C-Terminal Albumin-Angiostatin Expression Plasmid

An oligonucleotide pair was designed to manufacture a junction site between rHA and the angiostatin cDNA. The oligonucleotide pair JH021 and JH022 was designed to link the Bsu36I site within rHA to the BmrI site within the 5′ region of angiostatin.

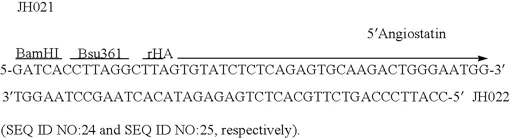

Plasmid pDB2447 was partially digested with BmrI and then digested to completion with BamHI to create a 3.95 kb vector. The double stranded oligonucleotide linker JH021/22 was ligated with the BamHI BmrI digested pDB2447 to create plasmid pDB2458. Plasmid pDB2458 was linearised with HindIII and treated with calf intestinal phosphatase to remove the 3′ phosphates. Plasmid pDB2441, described above, was digested to completion with HindIII and the 0.37 kb mADH1 terminator fragment was isolated from a 1% (w/v) agarose TAE gel using a Gene Clean III Kit (BIO0101 Inc.). The 0.37 kb HindIII mADH1 terminator fragment was ligated with the HindIII linearised pDB2458 to create plasmid pDB2459.

The DNA sequence of the human angiostatin cDNA encodes for one potential N-linked glycosylated site. The site for N-linked glycosylation was abolished by PCR mutagenesis. PCR primers JH025 and JH026 were designed to introduce a change within the nucleotide sequence to substitute an asparagine residue (codon AAC) with a glutamine residue (codon CAA).

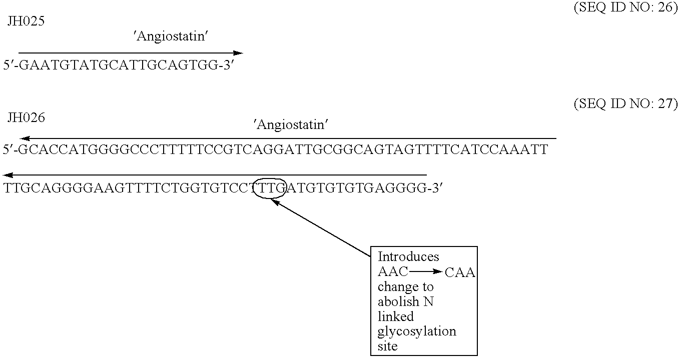

A master mix was prepared as follows: 2 mM MgCl₂ PCR Buffer, 10 μM PCR dNTP's, 0.2 μM JH025, 0.2 μM JH026, 2U FastStart Taq DNA polymerase (Roche). 1 μL of pDB2447 (10 pg, 100 pg, 1 ng, 10 ng, 100 ng) was added to 49 μL of reaction mix. The total reaction volume was 50 μL. Perkin-Elmer Thermal Cycler 9600 was programmed as follows: denature at 95° C. for 4 mins [HOLD]), then [CYCLE] denature at 95° C. for 30 s, anneal for 30 s at 45° C., extend at 72° C. for 60 s for 20 cycles, followed by a [HOLD] 72° C. for 600 s and then [HOLD] 4° C. The products of the PCR amplification were analysed by gel electrophoresis and a single DNA band of the expected size (0.46 kb) was observed. The modified angiostatin cDNA fragment was isolated from the 1% (w/v) agarose TAE gel using a Gene Clean III Kit (BIO101 Inc.). The non-glycosylated angiostatin cDNA fragment was digested to completion with NsiI, NcoI and the isolated 0.441 kb and ligated with the 3.93 kb NsiI, NcoI pDB2459 to create plasmid pDB2480.

Plasmid pDB2243, previously described in patent application WO 00/44772, which contained the yeast PRB1 promoter and the yeast ADH1 terminator, provided appropriate transcription promoter and transcription terminator sequences. Plasmid pDB2244, was digested to completion with BamHI, Bsu36I and the 5.84 kb fragment was isolated and ligated with the BamHI, Bsu36I angiostatin-mADH1 term fragment from pDB2480 to create pDB2501. Plasmid pDB2501 was digested with restriction endonuclease NotI to create a non-glycosylated albumin-angiostatin expression cassette.

Appropriate yeast vector sequences were provide by a “disintegration” plasmid pSAC35 generally disclosed in EP-A-286 424 and described by Sleep, D., et al. (1991) Bio/Technology 9, 183-187. The NotI C-terminal non-glycosylated albumin-angiostatin expression cassette was isolated from pDB2501, purified and ligated into NotI digested pSAC35 which had been treated with calf intestinal phosphatase, to create plasmid pDB2508 containing the NotI expression cassette in the same expression orientation as the LEU2 selectable marker and pDB2509 containing the NotI expression cassette in the opposite expression orientation as the LEU2 selectable marker.

Construction of N-Terminal Angiostatin-Albumin Expression Plasmid

The non-glycosylated angiostatin cDNA was modified by mutagenic PCR with two primers CF96 and CF97.

| | |
|---|---|
| CF96 | |
| 5′-CGATAGATCTTTGGATAAGAGAGTGTATCTCTCAGAGTGCAAGACTGG | (SEQ ID NO:28) |
| GAATGG-3′ | |
| CF97 | |
| 5′-GGCCATCGATGAGCGACTTCGGACTTGTGAGCGTCTACTGGGGAGGAGTCAC | (SEQ ID NO:29) |
| AGGACGG-3′ | |

A master mix was prepared as follows: 2 mM MgCl₂ PCR Buffer, 10 μM PCR dNTP's, 0.2 μM CF96, 0.2 μM CF97, 2U FastStart Taq DNA polymerase (Roche). 1 μL of pDB2501 (10 pg, 100 pg, 1 ng, 10 ng, 10 ng) was added to 49 μL of reaction mix. The total reaction volume was 50 μL. Perkin-Elmer Thermal Cycler 9600 was programmed as follows: denature at 95° C. for 4 mins [HOLD], then [CYCLE] denature at 95° C. for 30 s, anneal for 30 s at 55° C., extend at 72° C. for 90 s for 25 cycles, followed by a [HOLD] 72° C. for 600 s and then [HOLD] 4° C. The products of the PCR amplification were analysed by gel electrophoresis and a single DNA band of the expected size (0.83 kb) was observed. The modified angiostatin cDNA fragment was isolated from the 1% (w/v) agarose TAE gel using a Gene Clean III Kit (BIO101 Inc.). The non-glycosylated angiostatin cDNA fragment was digested to completion with BglI, ClaI and the isolated 0.83 kb and ligated with the 6.15 kb BglI, ClaI pDB2541 to create plasmid pDB2763.

Appropriate yeast vector sequences were provide by a “disintegration” plasmid pSAC35 generally disclosed in EP-A-286 424 and described by Sleep, D., et al. (1991) Bio/Technology 9, 183-187. The NotI N-terminal non-glycosylated angiostatin-albumin expression cassette was isolated from pDB2763, purified and ligated into NotI digested pSAC35 which had been treated with calf intestinal phosphatase, to create plasmid pDB2765 containing the NotI expression cassette in the same expression orientation as the LEU2 selectable marker and pDB2764 containing the NotI expression cassette in the opposite expression orientation as the LEU2 selectable marker.

### Example 5

### Construction of N-Terminal and C-Terminal Albumin-Kringle5 Fusions

Construction of C-Terminal Albumin-(GGS)₄GG-Kringle5 Expression Plasmid

Cloning of plasminogen Kringle5 for the C-terminal albumin fusion initiated with a PCR amplification of a human liver cDNA library (Ambion) using forward primer 5′-TGTATGTTTGGGAATGGGAAAG-3′ and reverse primer 5′-ACACTGAGGGACATCACAGTAG-3′ under standard conditions. A subsequent nested PCR using forward primer 5′-GTGGGATCCGGTGGTTGTATGTTTGGGAATGGGAAAG-3′ and reverse primer 5′-CACAAGCTTATTAACACTGAGGGACATCACAGTAG-3′ generated a DNA fragment which was subsequently cloned into pCR4-TA-TOPO (Invitrogen) according to the manufacturer's instructions. The resulting plasmid was called pCR4-Kringle5-C. The C-terminal Kringle5 DNA fragment was isolated from pCR4-Kringle5-C by digestion with BamHI and HindIII. Plasmid pDB2575 was partially digested with HindIII and then digested to completion with BamHI. The desired 6.55 kb DNA fragment was isolated and ligated with the 0.26 kb BamHI/HindIII fragment from plasmid pCR4-Kringle5-C to create plasmid pDB2717.

Appropriate yeast vector sequences were provide by a “disintegration” plasmid pSAC35 generally disclosed in EP-A-286 424 and described by Sleep, D., et al. (1991) Bio/Technology 9, 183-187. Plasmid pDB2717 was digested to completion with NotI and the 3.27 kb C-terminal albumin-(GGS)₄GG-kringle5 expression cassette isolated and subsequently ligated into NotI calf intestinal phosphatase treated pSAC35 to create plasmid pDB2748 containing the NotI expression cassette in the same expression orientation as the LEU2 selectable marker and pDB2749 containing the NotI expression cassette in the opposite expression orientation as the LEU2 selectable marker.

Construction of N-Terminal Kringle5-(GGS)₄GG-Albumin Expression Plasmid

Cloning of plasminogen Kringle5 for the N-terminal albumin fusion was performed by PCR amplification of the Kringle5 sequence contained in clone pCR4-Kringle5-C, using forward primer 5′-GTGAGATCTTGTATGTTTGGGAATGGGAAAG-3′ and reverse primer 5′-CACGGATCCACCACACTGAGGGACATCACAGTAG-3′ under standard conditions The amplified DNA fragment was digested with restriction endonucleases BglII and BamHI and cloned into pLITMUS29 (New England BioLabs). The resulting plasmid was called pCR4-Kringle5-N. Plasmid pCR4—Kringle5-N was digested to completion with BamHI and BglII. The 0.26 kb DNA fragment was ligated into BamHI, BglII digested pDB2573 to create plasmid pDB2771. Appropriate yeast vector sequences were provide by a “disintegration” plasmid pSAC35 generally disclosed in EP-A-286 424 and described by Sleep, D., et al. (1991) Bio/Technology 9, 183-187. Plasmid pDB2771 was digested to completion with NotI and the 3.27 kb N-terminal Kringle5-(GGS)₄GG-albumin expression cassette isolated and subsequently ligated into NotI calf intestinal phosphatase treated pSAC35 to create plasmid pDB2773 containing the NotI expression cassette in the same expression orientation as the LEU2 selectable marker and pDB2774 containing the NotI expression cassette in the opposite expression orientation as the LEU2 selectable marker.

### Example 6

### Yeast Transformation and Culturing Conditions

Yeast strains disclosed in WO 95/23857, WO 95/33833 and WO 94/04687 were transformed to leucine prototrophy as described in Sleep D., et al. (2001) Yeast 18, 403-421. The transformants were patched out onto Buffered Minimal Medium (BMM, described by Kerry-Williams, S. M. et al. (1998) Yeast 14, 161-169) and incubated at 30° C. until grown sufficiently for further analysis.

### Example 7

### Expression of Albumin Endostatin Fusion Proteins

rHA fusions were expressed in a shake flask and the culture expression level was measured. For rHA-endostatin, expression level in the culture supernatant was high. For endostatin-rHA, expression was medium high in the culture supernatant.

### Example 8

### Purification of Albumin Endostatin Fusions

C-Terminal Endostatin Purification:

The C-terminal endostatin was purified using the standard rHA SP-FF (Pharmacia) conditions as described in WO 00/44772, except it required an extra 250 mM NaCl in the elution buffer. The eluate was then purified using standard rHA DE-FF (Pharmacia) conditions as described in WO 00/44772, except that an extra 200 mM NaCl was included in the elution buffer (although this salt concentration was not optimized and, therefore, may be varied). The purified material was then concentrated and diafiltered against PBS.

N-Terminal Endostatin Purification:

The N-terminal endostatin was purified using the standard rHA SP-FF conditions, except it required an extra 250 mM NaCl in the elution buffer. The eluate was then adjusted to pH 8 and 2.5 mS·cm⁻¹ and purified using standard rHA DE-FF equilibrated in 15 mM potassium tetraborate. The DE-FF was eluted using the standard rHA elution buffer. The purified material was then concentrated and diafiltered against PBS.

The fermentation titres were 2.2 and 0.9 mg·mL⁻¹ for the C and N terminal fusions respectively and the overall purification recovery was high. It may be possible to both further improve the purification recovery, depending on purity required, and increase the fermentation titre, particularly for the N-terminal fusion.

### Example 9

### Characterization of Albumin Endostatin Fusions

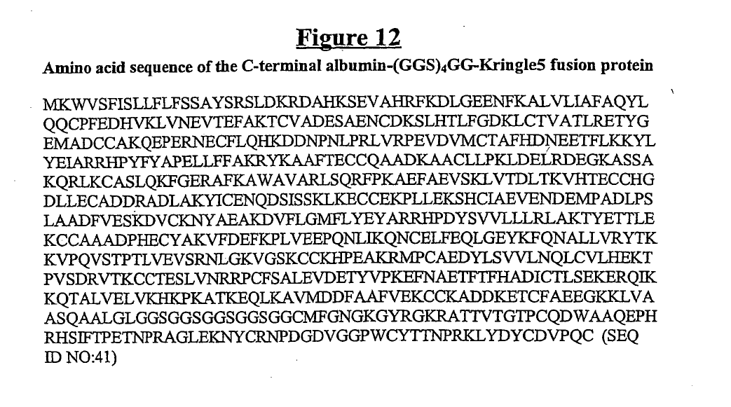

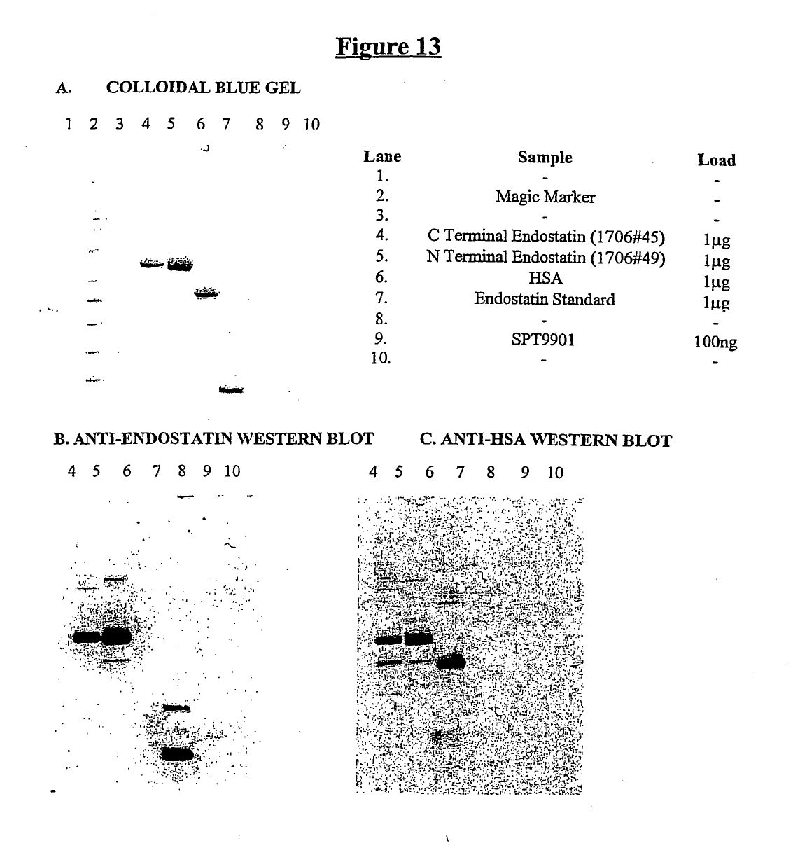

The protein after purification was characterized by running the sample on a 4-12% gradient SDS non-reducing gel and performing a Western blot with anti-endostatin or anti-HSA antibodies. The results are shown in FIG. 13. The gel was loaded as follows:

| Lane | Sample | Load |
|---|---|---|
| 1. | — | — |
| 2. | Magic Marker | — |
| 3. | — | — |
| 4. | C Terminal Endostatin | 1 μg |
| 5. | N Terminal Endostatin | 1 μg |
| 6. | HSA | 1 μg |
| 7. | Endostatin Standard | 1 μg |

The protein as characterized in the following table:

TABLE 3
Protein Characterization After Purification

| | C-Terminal Fusion | N-Terminal Fusion |
|---|---|---|
| % Purity by SDS- | 95 | 99 |
| PAGE and colloidal | | |
| blue staining | | |
| ESMS indication of | No species of correct | A species of correct |
| post-translational | theoretical mass = 86512 | theoretical mass was |
| modifications | detected. | detected. Some |
| | Main species | higher mol weight |
| | consistent with loss | components present.² |
| | of CT lysine residue.¹ | |
| N-Terminal Sequence | Correct NT sequence | Correct NT sequence |
| | for rHA | for Endostatin |
| Endotoxin | 4.3 | 5.7 |
| (EU · mL⁻¹) | | |
| Fusion Concentration | 5 | 5 |
| (mg · mL⁻¹) | | |

Notes:
¹Essentially a single peak. The loss of the CT lysine residue, observed in three different preps, was confirmed by nano-MS of the tryptic peptides.
²Good evidence for correct unprocessed primary sequence. Additional species at +78 and +165 Da observed, possibly phosphorylation and glycosylation respectively. +78 not observed in C-term preparations.

### Example 10

### Pharmacokinetics of Albumin Endostatin Fusion Proteins

Endostatin antigen levels were measured in mouse serum after i.v. or s.c. injection of endostatin, C-terminal albumin-fusion with endostatin (CT-endostatin) or N-terminal albumin-fusion with endostatin (NT-endostatin).

Mice received a single injection of the test substance. At each sample point 5 mice per group were bled and serum was collected for ELISA analysis.

PK Data:

Data for CT- and NT-endostatin after s.c. and i.v. application compared to “classic” endostatin show similar results:

Endostatin (classic): 4.5 hrs

CT-endostatin: 56 hrs

NT-endostatin: 29 hrs

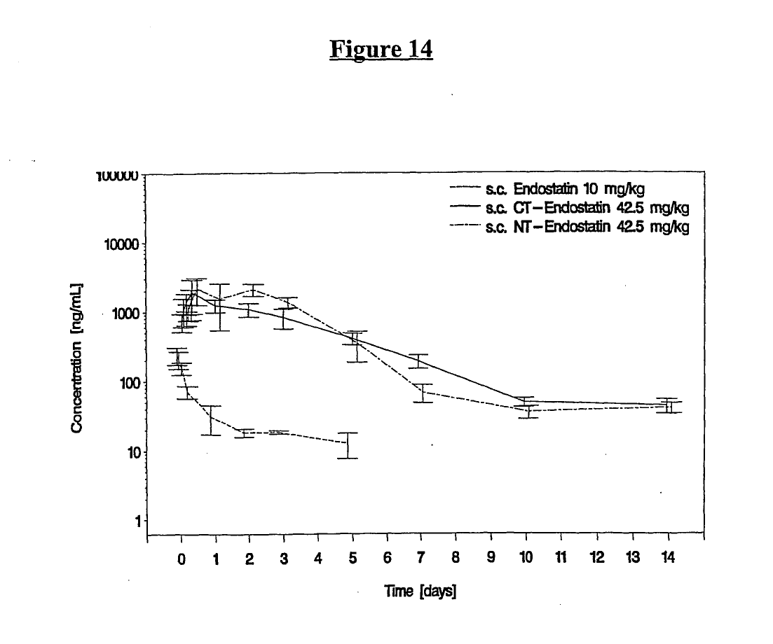

Table 4 shows the pharmacokinetic results following s.c. administration. Mean endostatin concentrations, +/−S.D., following s.c. application are shown in FIG. 14.

TABLE 4
Pharmacokinetic results following s.c. administration

| | | | | Endostatin |
|---|---|---|---|---|
| | Endostatin | CT- | NT- | 1.25 mg/ |
| | 10 mg/kg | Endostatin | Endostatin | kg |
| Absorption half-life | 0.09 | 1.61 | 8.84 | 0.05 |
| (hr) | | | | |
| Terminal half-life | 4.5 | 55.7 | 28.4 | 2.0^{a} |
| (hr) | | | | |
| AUC (hr · ng/mL) | 3,010 | 142,183 | 175,272 | 2,682^{b} |
| Cₘₐₓ (ng/mL) | 229 | 1,785 | 2,198 | 44 |

^{a}Calculated from values up to 24 hours
^{b}Area includes increasing levels after 24 hours

Table 5 shows the pharmacokinetic results following i.v. administration. FIG. 15 shows the mean endostatin concentration, +/−S.D., following i.v. application.

TABLE 5
Pharmacokinetic results following i.v. administration

| | Endostatin | | |
|---|---|---|---|
| | 1.25 mg/kg | CT-Endostatin | NT-Endostatin |
| Initial half-life (hr) | — | 6.39 | 2.40 |
| Terminal half-life (hr) | 1.9 | 50.0 | 23.7 |
| AUC (hr · ng/mL) | 1,723 | 456,139 | 658,469 |
| Cₘₐₓ (ng/mL) | 126 | 24,252 | 24,127 |

Obtained data were used to simulate repeated dosing as needed in a 21 day efficacy trial. An accumulation study suggested four dosing shedules to stay within the favourable therapy window of 150-400 ng/ml. A PK study to test repeated dosing of AFP-endostatins was performed to clarify this issue. Four dosing shedules were tested, as set forth in Table 6, below.

TABLE 6
Dosing Schedules For Repeated Dosing of AFP-endostatins

| | | Loading dose/schedule of |
|---|---|---|
| No. | Treatment | maintenance dose/route |
| 1 | CT-Endostatin 72 h | 1.8 mg/kg/1.2 mg/kg every 72 h/s.c. |
| 2 | CT-Endostatin 24 h | 1.5 mg/kg/0.5 mg/kg every 24 h/s.c. |
| 3 | NT-Endostatin 72 h | 1.0 mg/kg/0.9 mg/kg every 72 h/s.c. |
| 4 | NT-Endostatin 24 h | 0.8 mg/kg/0.25 mg/kg every 24 h/s.c. |

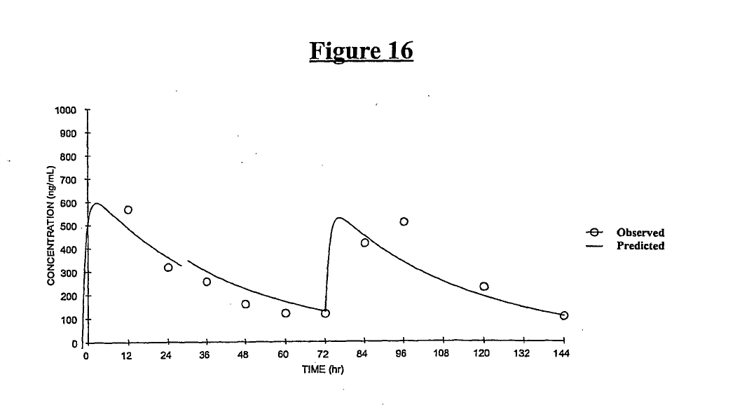

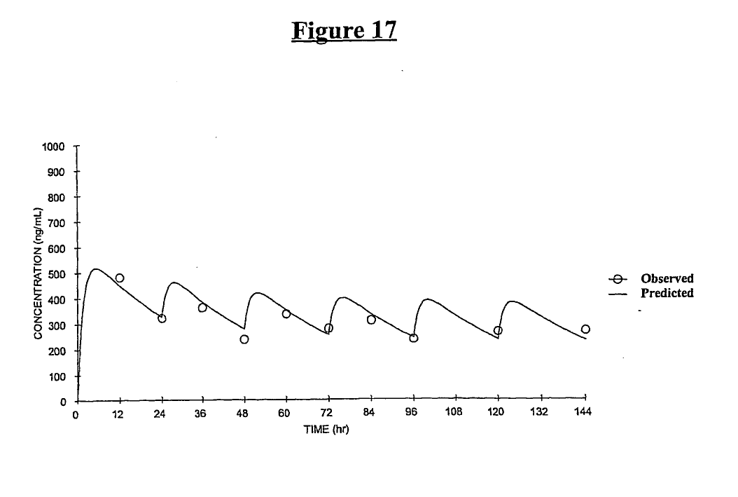

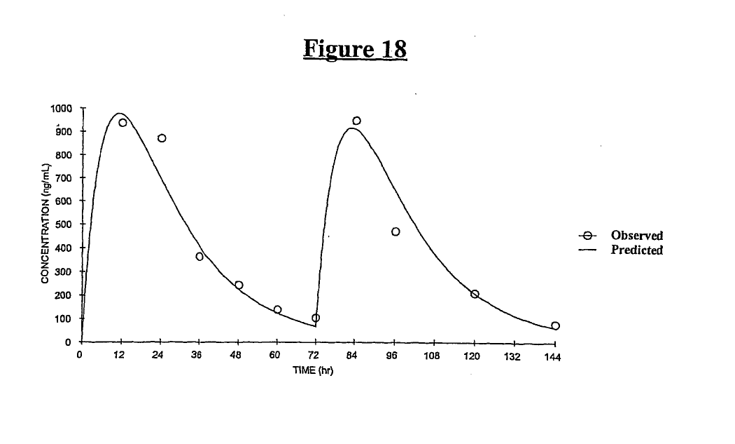

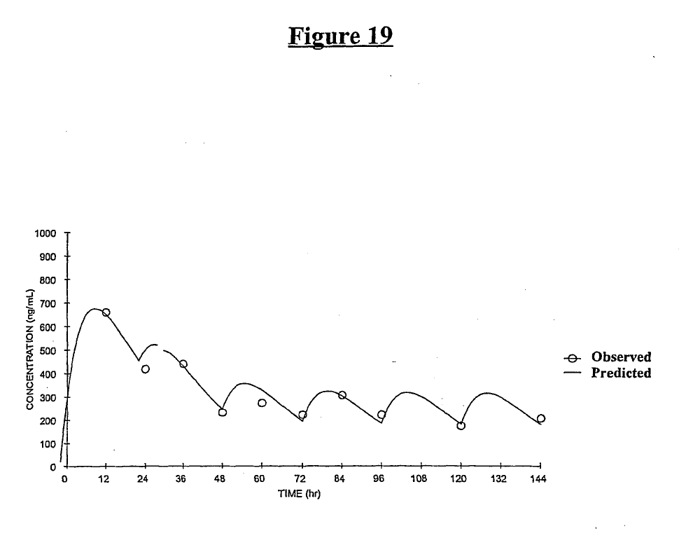

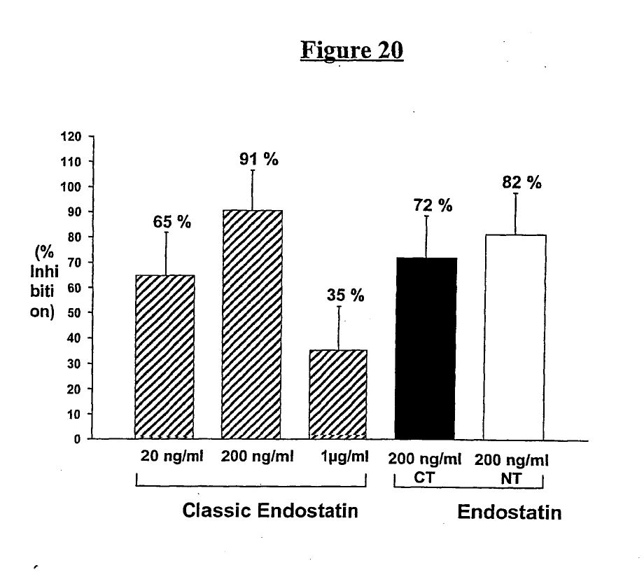

The pharmacokinetic results following multiple s.c. administration are shown in Table 7 and in FIGS. 16 to 19.

TABLE 7
Pharmacokinetic results following multiple s.c. administrations

| | CT- | CT- | NT- | NT- |
|---|---|---|---|---|
| | Endostatin | Endostatin | Endostatin | Endostatin |
| | 72 h | 24 h | 72 h | 24 h |
| Absorption half-life (hr) | 0.85 | 1.12 | 4.69 | 5.30 |
| Terminal half-life (hr) | 29.1 | 25.5 | 13.7 | 10.7 |
| Cₘₐₓ (ng/mL)^{a} | 568 | 481 | 937 | 659 |
| tₘₐₓ (hr)^{a} | 12 | 12 | 12 | 12 |

^{a}following the first dose

### Example 11

### In Vitro Efficacy of Albumin Endostatin Fusion Proteins

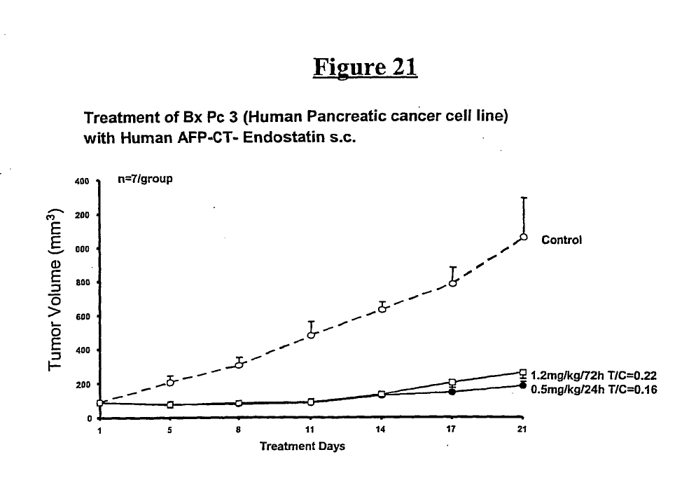

CT-endostatin and NT-endostatin show similar efficacy compared to classic endostatin in an in vitro migration-assay (HUVEC). These results are shown in FIG. 20.

### Example 12

### In Vivo Efficacy of Albumin Endostatin Fusion Proteins

CT-endostatin and NT-endostatin show similar efficacy in vivo, in a pancreas tumor model in mice, as compared to classic endostatin.

CT-endostatin shows in one dosage scheme better efficacy:
Dose response and tumor shrinkage in 2 out of 7 cases
3.6 mg/kg every 72 hrs instead of 100 mg/kg every 24 hrs for best classic data so far (Kisker et. al., Cancer Res. 61: 7669-7674 (2001))

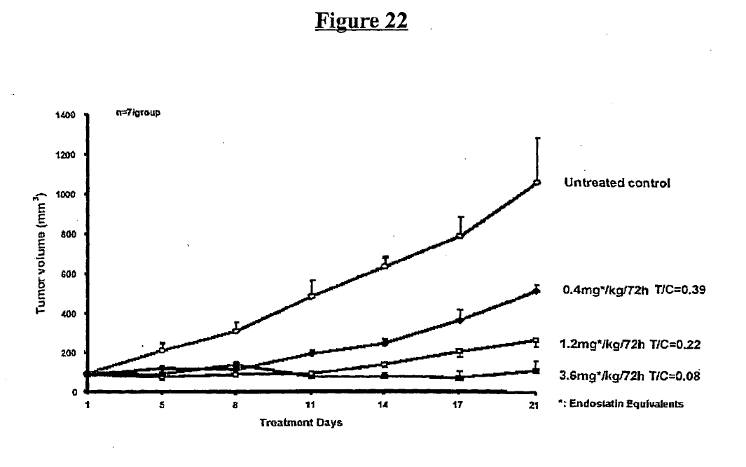

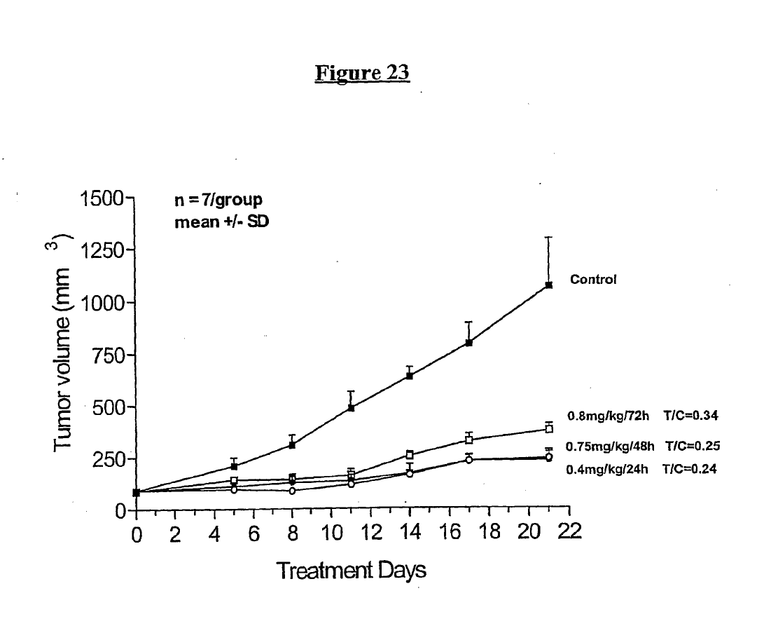

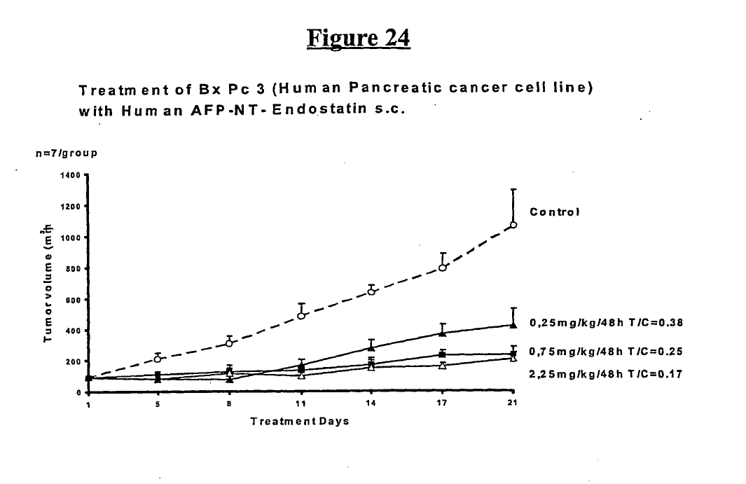

The results of treatment of Bx Pc3 (a human pancreatic cancer cell line) with CT-endostatin, s.c., are shown in FIGS. 21 to 24.

### Example 13

### Expression of Albumin Angiostatin Fusion Proteins

rHA fusions were expressed in shake flask culture and the expression levels were measured. The expression level in culture supernatant was low for rHA-angiostatin; rHA-3xFLAG-angiostatin; rHA-angiostatin (N211Q); and rHA-3xFLAG-angiostatin (N211Q); A SDS-PAGE gel of these fusions is shown in FIG. 27. The lanes were loaded as follows:

Lane Sample

1 rHA-3xFLAG-angiostatin(N211Q)

2 rHA-angiostatin(N211Q)

3 rHA-angiostatin

4 rHA-angiostatin

5 rHA

### Example 14

### Purification of Albumin Angiostatin Fusion Proteins

C-Terminal Angiostatin Purification

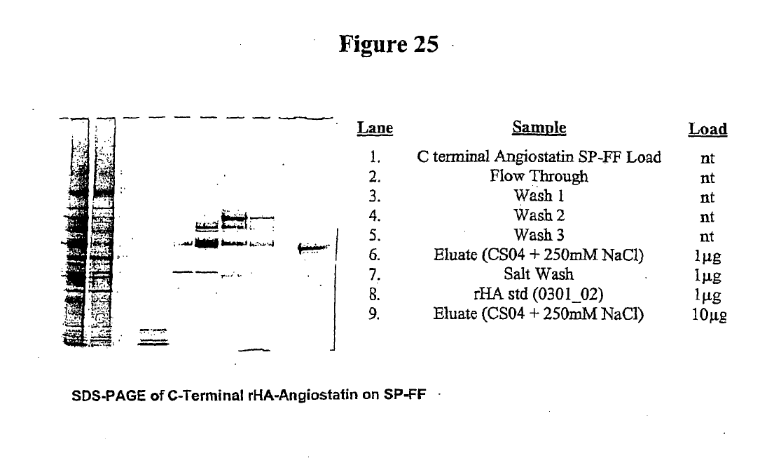

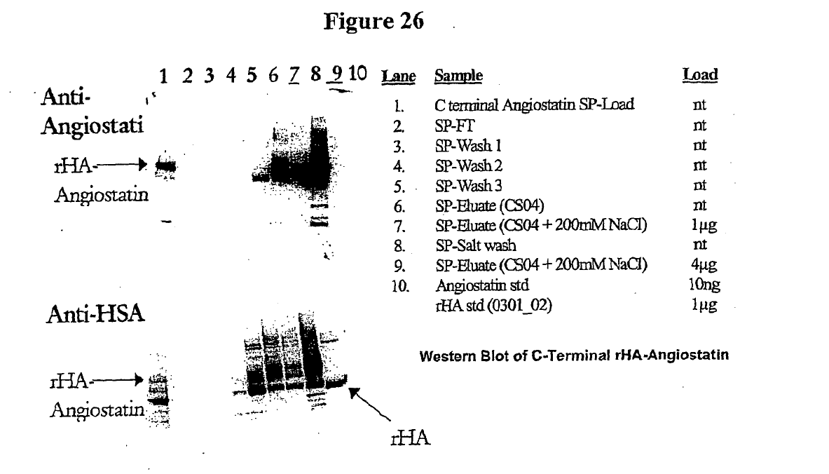

The C-terminal angiostatin contained high levels of clipped material. It was purified using the standard rHA SP-FF conditions using the normal elution as a wash and eluting using the standard buffer containing 200 mM NaCl. The eluate of the SP-FF column was analyzed by SDS-PAGE as shown in FIG. 25. Western blots of SP-FF eluates using anti-angiostatin or anti-HSA antibodies are shown in FIG. 26. The eluate was then purified using standard rHA DE-FF conditions, except it required an extra 10 mM NaCl in the elution buffer. The purified material was then concentrated and diafiltered against PBS.

The purification performed for this fusion protein forms a good basis for a production process but would require further work to in order to analyse yeast antigen clearance and optimise recoveries. The final amounts produced were low, but this was mainly due to the fermentation titre of less than 0.1 mg·mL⁻¹, rather than poor recoveries. Recoveries were generally good but could be improved across the DE-FF depending on purity required. However, if the overall yield needed to be increased, the greatest gain would be from increasing the expression levels.

### REFERENCES

Cao et al, (1996) J. Biol. Chem. 271(46):29461-29467
Cao et al, (1997) J. Biol. Chem. 272(36):22924-22928
Dhanabal (1999) Cancer Research 59:189-197
Folkman, J. (1971) New England Journal of Medicine 285:1182-1186
Kerry-Williams, S. M. et al. (1998) Yeast 14, 161-169
Kisker et. al., Cancer Res. 61: 7669-7674 (2001)
Lu et al; (1999) Biochem. Biophysical Research Cummunications 258:668-673
O'Reilly, M. (1997) Cell. 88:277-285
Sim et al. (2000) Cancer and Metastasis Reviews 19:181-190
Sleep, D., et al. (1991) Bio/Technology 9, 183-187
Sleep D., et al. (2001) Yeast 18, 403-421
EP-A-286 424
UK 0217033.0
U.S. Pat. No. 5,792,845
U.S. Pat. No. 5,854,205
U.S. Pat. No. 5,854,221
U.S. Pat. No. 5,885,795
WO 94/04687
WO 95/23857
WO 95/33833
WO 97/15666
WO 00/44772

The present invention is not to be limited in scope by the specific embodiments described which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

Every reference cited hereinabove is incorporated by reference in its entirety.

1. An albumin fusion protein comprising an angiogenesis inhibiting peptide, or a fragment or variant thereof, and albumin, or a fragment or variant thereof, wherein
said albumin, or fragment or variant thereof.

2. The albumin fusion protein of claim 1 comprising at least one endostatin, or a fragment or variant thereof.

3. The albumin fusion protein of claim 1 comprising at least one angiostatin, or a fragment or variant thereof.

4. The albumin fusion protein of claim 1 comprising at least one Kringle 5, or a fragment or variant thereof.

5. The albumin fusion protein of claim 1 wherein the albumin fusion protein comprising at least two angiogenesis inhibiting peptides or fragments or variants thereof.

6. The albumin fusion protein of claim 5 wherein at least two of the angiogenesis inhibiting peptides or fragments or variants thereof have different amino acid sequences.

7. The albumin fusion protein of claim 5 which comprises a first angiogenesis inhibiting peptide, or fragment or variant thereof, and a second angiogenesis inhibiting peptide, or fragment or variant thereof, wherein said fast angiogenesis fusion inhibiting peptide, or fragment or variant thereof, is different from said second angiogenesis fusion inhibiting peptide, or fragment or variant thereof.

8. The albumin fusion protein of claim 1 wherein said albumin or fragment or variant thereof has the ability to prolong the in vivo half-life of the angiogenesis inhibiting peptide, or a fragment or variant thereof, compared to the in vivo half-life of the angiogenesis inhibiting peptide, or a fragment or variant thereof, in an unfused state.

9. The albumin fusion protein of claim 1 further comprising one or more additional angiogenesis inhibiting peptide, or a fragment or variant thereof, or one or more additional albumin, or a fragment or variant thereof.

10. The albumin fusion protein of claim 1 wherein said fusion protein further comprises a chemical moiety.

11. The albumin fusion protein of claim 1 wherein the angiogenesis inhibiting peptide, or fragment or variant thereof, is fused to the N-terminus of albumin, or the N-terminus of the fragment or variant of albumin.

12. The albumin fusion protein of claim 1 wherein angiogenesis inhibiting peptide, or fragment or variant thereof, is fused to the C-terminus of albumin, or the C-terminus of the fragment or variant of albumin.

13. The albumin fusion protein of claim 1 wherein angiogenesis inhibiting peptide, or fragment or variant thereof, is fused to an internal region of albumin, or an internal region of a fragment or variant of albumin.

14. The albumin fusion protein of claim 1 wherein the angiogenesis inhibiting peptide, or fragment or variant thereof, is separated from the albumin or the fragment or variant of albumin by a linker.

15. The albumin fusion protein of claim 1 wherein the albumin fusion protein comprises the following formula:
R2-R1; R1-R2; R2-R1-R2; R2-L-R1-L-R2; R1-L-R2; R2-L-R1; or R1-L-R2-L-R1,
wherein R1 is at least one Therapeutic protein, peptide or polypeptide sequence,
including fragments or variants thereof, and not necessarily the same Therapeutic protein, L is a linker and R2 is a serum albumin sequence, including fragments or variants thereof.

16. The albumin fusion protein of claim 1 wherein the in vivo-half-life of the albumin fusion protein is greater than the in vivo half-life of the angiogenesis inhibiting peptide in an unfused state.

17. The albumin fusion protein of claim 1 wherein the in vitro biological activity of the angiogenesis inhibiting peptide, or fragment or variant thereof, fused to albumin, or fragment or variant thereof, is greater than the in vitro biological activity of the angiogenesis inhibiting peptide, or fragment or variant thereof, in an unfused state.

18. The albumin fusion protein of claim 1 wherein the in vivo biological activity of the angiogenesis inhibiting peptide, or fragment or variant thereof, fused to albumin, or fragment or variant thereof, is greater than the in vivo biological activity of the angiogenesis inhibiting peptide, or fragment or variant thereof, in an unfused state.

19. The albumin fusion protein of claim 1 which is expressed in yeast.

20. The albumin fusion protein of claim 19 wherein the yeast is glycosylation deficient.

21. The albumin fusion protein of claim 19 wherein the yeast is glycosylation and protease deficient.

22. The albumin fusion protein of claim 1 which is expressed by a mammalian cell.

23. The albumin fusion protein of claim 1 wherein the albumin fusion protein is expressed by a mammalian cell in culture.

24. A composition comprising the albumin fusion protein of claim 1 and a carrier.

25. A pharmaceutical composition comprising an effective amount of the albumin fusion protein of claim 1 and a pharmaceutically acceptable carrier or excipient.

26. A method of treating an angiogenesis-related disease or disorder in a patient, comprising the step of administering an effective amount of the albumin fusion protein of claim 1.

27. A method of treating a patient with a solid tumor or hematological cancer that is treatable by angiogenesis inhibiting peptide, comprising the step of administering an effective amount of the albumin fusion protein of claim 1.

28. A method of extending the in vivo half-life of angiogenesis inhibiting peptide, or a fragment or variant thereof, comprising the step of fusing the angiogenesis inhibiting peptide, or fragment or variant thereof, to albumin or a fragment or variant of albumin sufficient to extend the in vivo half-life of the angiogenesis inhibiting peptide, or fragment or variant thereof, compared to the in vivo half-life of the angiogenesis inhibiting peptide, or fragment or variant thereof, in an unfused state.

29. A method for extending the half-life of angiogenesis inhibiting peptide in a mammal, the method comprising linking said angiogenesis inhibiting peptide to an albumin to form an albumin-fused angiogenesis inhibiting peptide and administering said albumin-fused angiogenesis inhibiting peptide to said mammal, whereby the half-life of said albumin-fused angiogenesis inhibiting peptide is extended at least 2-fold over the half-life of the angiogenesis inhibiting peptide lacking the linked albumin.

30. A nucleic acid molecule comprising a polynucleotide sequence encoding the albumin fusion protein of claim 1.

31. A vector comprising the nucleic acid molecule of claim 30.

32. A host cell containing the nucleic acid molecule of claim 30.

33. A method for minimizing a side effect associated with the treatment of a mammal with angiogenesis inhibiting peptide, the method comprising administering an albumin-fused angiogenesis inhibiting peptide or a nucleic acid capable of expressing an effective concentration of said albumin fusion protein of claim 1 to said mammal.

34. A method for manufacturing an albumin fusion protein of claim 1, the method comprising (a) providing a nucleic acid comprising a nucleotide sequence encoding the albumin fusion protein expressible in a cell or organism; (b) expressing the nucleic acid in the cell or organism to form an albumin fusion protein; and (c) purifying the albumin fusion protein.

35. The method of claim 34 wherein the albumin fusion protein is expressed in a glycosylation deficient yeast strain.

36. The method of claim 34 wherein the peptide albumin fusion is expressed in a glycosylation competent yeast strain.

37. A composition comprising an albumin fusion protein of claim 1, wherein the albumin fusion protein is provided in amounts such that the composition is capable of effectively regressing the tumor mass of angiogenesis-dependent tumors when administered to patients with an angiogenesis-dependent tumor.

38. A composition comprising a fusion protein of claim 1 wherein the fusion protein is provided in amounts such that the composition is capable of effectively inhibiting growth of an angiogenesis-dependent tumor.

39. The method of claim 26 wherein the angiogenesis-related disease is angiogenesis-dependent cancer.

40. The method of claim 26 wherein the angiogenesis-related disease is selected from the group consisting of angiogenesis-dependent cancers; benign tumors; rheumatoid arthritis; psoriasis; ocular angiogenesis diseases; Osler-Webber Syndrome; myocardial angiogenesis; plaque neovascularization; telangiectasia; hemophiliac joints; angiofibroma; wound granulation; intestinal adhesions, atherosclerosis, scleroderma, hypertrophic scars, cat scratch disease and Helobacter pylori ulcers.

41. A method of treating a patient with an angiogenesis-dependent tumor comprising administering to a patient in need of such treatment of the albumin fusion protein of claim 1 or a nucleic acid capable of expressing an effective concentration of said albumin fusion protein of claim 1 in an amount sufficient to cause tumor regression.

42. A method of treating a patient with an angiogenesis-dependent tumor comprising administering to a patient in need of such treatment of the albumin fusion protein of claim 1 or a nucleic acid capable of expressing an effective concentration of said albumin fusion protein of claim 1 in an amount sufficient to cause tumor stasis.

43. A vaccine composition for inducing immunity in a mammal against an angiogenesis-dependent disease or disorder comprising a pharmaceutically acceptable carrier and a therapeutically effective amount of an albumin fusion protein of claim 1 or a nucleic acid capable of expressing an effective concentration of said albumin fusion protein of claim 1.

44. A vaccine composition according to claim 43 wherein said mammal is a human.

45. A method for inducing immunity against an angiogenesis-dependent cancer tumor in a mammal which comprises administering to a mammal a therapeutically effective amount of a vaccine composition according to claim 43.

46. A method according to claim 45 wherein said mammal is a human.

47. The albumin fusion protein of claim 1 further comprising a targeting portion adapted to target the albumin fusion protein to a cell type, target organ, or a specific cytological or anatomical location.

48. A method of diagnosing -an anti-.angiogenesis related disease or disorder in a mammal comprising
(a) administering a labeled fusion protein of claim 1;
(b) allowing at least some of the labeled fusion protein to reach the site of the angiogenesis dependent disease or disorder; and
(c) determining whether the fusion protein at the site of the angiogenesis dependent disease or disorder.

49. A method of targeting an antiangiogenic peptide to the inside of a cell or at cell structures in a mammal comprising fusing the peptide to albumin or a fragment or variant thereof to create a fusion protein and administering the fusion protein to a mammal.

50. A method of improving the scheduling of dosing of an antiangiogenic peptide comprising fusing the peptide to albumin or a fragment or variant thereof to create a fusion protein and administering the fusion protein to a mammal comprising
(a) dose optimization design on the basis of the angiogeneic phenotype of a tumor to fit specific growth characteristics of individual tumors; and
(b) controlling/avoiding unwanted accumulation of drug in longer applications which could result in fewer or lessened side reactions or altered efficacy.


SEQUENCE LISTING (47 sequences)

SEQ ID NO: 1 - protein, 20 aa, Mus sp.
HTHQDFQPVLHLVALNTPLS

SEQ ID NO: 2 - protein, 20 aa, Homo sapiens
HSHRDFQPVLHLVALNSPLS

SEQ ID NO: 3 - protein, 5 aa, Artificial Sequence
  Description of Artificial Sequence Exemplary linker
GGGGS

SEQ ID NO: 4 - protein, 4 aa, Artificial Sequence
  Description of Artificial Sequence Exemplary linker
GGGS

SEQ ID NO: 5 - protein, 17 aa, Unknown Organism
  Description of Unknown Organism Stanniocalcin signal sequence
MLQNSAVLLLLVISASA

SEQ ID NO: 6 - protein, 22 aa, Artificial Sequence
  Description of Artificial Sequence Consensus signal sequence
MPTWAWWLFLVLLLALWAPARG

SEQ ID NO: 7 - DNA, 43 bp, Artificial Sequence
  Description of Artificial Sequence Primer
tagcggatccacagccaccgcgacttccagccggtgctccacc

SEQ ID NO: 8 - DNA, 54 bp, Artificial Sequence
  Description of Artificial Sequence Primer
gctaaagcttattacttggaggcagtcatgaagctgttctcaatgcagagcacg

SEQ ID NO: 9 - DNA, 49 bp, Artificial Sequence
  Description of Artificial Sequence Synthetic oligonucleotide
ttaggcttacacagccaccgcgacttccagccggtgctccacctggtgt

SEQ ID NO: 10 - DNA, 48 bp, Artificial Sequence
  Description of Artificial Sequence Synthetic oligonucleotide
atacaccaggtggagcaccggctggaagtcgcggtggctgtgtaagcc

SEQ ID NO: 11 - DNA, 12 bp, Artificial Sequence
  Description of Artificial Sequence Synthetic oligonucleotide
tcgagaagcttc

SEQ ID NO: 12 - DNA, 48 bp, Artificial Sequence
  Description of Artificial Sequence Synthetic oligonucleotide
gatctttggataagagagacgctcacaagtccgaagtcgctcaccggt

SEQ ID NO: 13 - DNA, 50 bp, Artificial Sequence
  Description of Artificial Sequence Synthetic oligonucleotide
ccttgaaccggtgagcgacttcggacttgtgagcgtctctcttatccaaa

SEQ ID NO: 14 - DNA, 48 bp, Artificial Sequence
  Description of Artificial Sequence Synthetic oligonucleotide
gatctttggataagagagacgctcacaagtccgaagtcgctcatcgat

SEQ ID NO: 15 - DNA, 50 bp, Artificial Sequence
  Description of Artificial Sequence Synthetic oligonucleotide
ccttgaatcgatgagcgacttcggacttgtgagcgtctctcttatccaaa

SEQ ID NO: 16 - DNA, 86 bp, Artificial Sequence
  Description of Artificial Sequence Synthetic oligonucleotide
tcaaggacctaggtgaggaaaacttcaaggctttggtcttgatcgctttcgctcaatacttgcaacaatgtccattcgaagatcac

SEQ ID NO: 17 - DNA, 1782 bp, Homo sapiens
  CDS (1)..(1755)
gatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtggctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaggattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaactgtgagctttttgagcagcttggagagtacaaattccagaatgcgctattagttcgttacaccaagaaagtaccccadgtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcacaaaatgctgcacagagtccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataacatctacatttaaaagcatctcag

SEQ ID NO: 18 - protein, 585 aa, Homo sapiens [sequence omitted]

SEQ ID NO: 19 - DNA, 80 bp, Artificial Sequence
  Description of Artificial Sequence Synthetic oligonucleotide
gtgatcttcgaatggacattgttgcaagtattgagcgaaagcgatcaagaccaaagccttgaagttttcctcacctaggt

SEQ ID NO: 20 - DNA, 57 bp, Artificial Sequence
  Description of Artificial Sequence Synthetic oligonuclentide
ctctagatctttggataagagacacagccaccgcgacttccagccggtgctccacct

SEQ ID NO: 21 - DNA, 70 bp, Artificial Sequence
  Description of Artificial Sequence Synthetic oligonucleotide
ccttgaaccggtgagcgacttcggacttgtgagcgtccttggaggcagtcatgaagctgttctcaatgca

SEQ ID NO: 22 - DNA, 53 bp, Artificial Sequence
  Description of Artificial Sequence Primer
ggagtactgtaagataccgtcctgtgactcctccccagtataataagcttttt

SEQ ID NO: 23 - DNA, 49 bp, Artificial Sequence
  Description of Artificial Sequence Primer
tagcggatccgtgtatctctcagagtgcaagactgggaatggaaagaac

SEQ ID NO: 24 - DNA, 48 bp, Artificial Sequence
  Description of Artificial Sequence Synthetic oligonucleotide
gatcaccttaggcttagtgtatctctcagagtgcaagactgggaatgg

SEQ ID NO: 25 - DNA, 44 bp, Artificial Sequence
  Description of Artificial Sequence Synthetic oligonucleotide
ccattcccagtcttgcactctgagagatacactaagcctaaggt

SEQ ID NO: 26 - DNA, 20 bp, Artificial Sequence
  Description of Artificial Sequence Primer
gaatgtatgcattgcagtgg

SEQ ID NO: 27 - DNA, 98 bp, Artificial Sequence
  Description of Artificial Sequence Primer
gcaccatggggccctttttccgtcaggattgcggcagtagttttcatccaaatttttgcaggggaagttttctggtgtcctttgatgtgtgtgagggg

SEQ ID NO: 28 - DNA, 54 bp, Artificial Sequence
  Description of Artificial Sequence Primer
cgatagatctttggataagagagtgtatctctcagagtgcaagactgggaatgg

SEQ ID NO: 29 - DNA, 59 bp, Artificial Sequence
  Description of Artificial Sequence Primer
ggccatcgatgagcgacttcggacttgtgagcgtctactggggaggagtcacaggacgg

SEQ ID NO: 30 - DNA, 2376 bp, Artificial Sequence
  Description of Artificial Sequence DNA sequence of the N terminal endostatin-albumin fusion open reading frame
atgaagtgggttttcatcgtctccattttgttcttgttctcctctgcttactctagatctttggataagagacacagccaccgcgacttccagccggtgctccacctggttgcgctcaacagccccctgtcaggcggcatgcggggcatccgcggggccgacttccagtgcttccagcaggcgcgggccgtggggctggcgggcaccttccgcgccttcctgtcctcgcgcctgcaggacctgtacagcatcgtgcgccgtgccgaccgcgcagccgtgcccatcgtcaacctcaaggacgagctgctgtttcccagctgggaggctctgttctcaggctctgagggtccgctgaagcccggggcacgcatcttctcctttgacggcaaggacgtcctgaggcaccccacctggccccagaagagcgtgtggcatggctcggaccccaacgggcgcaggctgaccgagagctactgtgagacgtggcggacggaggctccctcggccacgggccaggcctcctcgctgctggggggcaggctcctggggcagagtgccgcgagctgccatcacgcctacatcgtgctctgcattgagaacagcttcatgactgcctccaaggacgctcacaagtccgaagtcgctcaccggttcaaggacctaggtgaggaaaacttcaaggctttggtcttgatcgctttcgctcaatacttgcaacaatgtccattcgaagatcacgtcaagttggtcaacgaagttaccgaattcgctaagacttgtgttgctgacgaatctgctgaaaactgtgacaagtccttgcacaccttgttcggtgataagttgtgtactgttgctaccttgagagaaacctacggtgaaatggctgactgttgtgctaagcaagaaccagaaagaaacgaatgtttcttgcaacacaaggacgacaacccaaacttgccaagattggttagaccagaagttgacgtcatgtgtactgctttccacgacaacgaagaaaccttcttgaagaagtacttgtacgaaattgctagaagacacccatacttctacgctccagaattgttgttcttcgctaagagatacaaggctgctttcaccgaatgttgtcaagctgctgataaggctgcttgtttgttgccaaagttggatgaattgagagacgaaggtaaggcttcttccgctaagcaaagattgaagtgtgcttccttgcaaaagttcggtgaaagagctttcaaggcttgggctgtcgctagattgtctcaaagattcccaaaggctgaattcgctgaagtttctaagttggttactgacttgactaaggttcacactgaatgttgtcacggtgacttgttggaatgtgctgatgacagagctgacttggctaagtacatctgtgaaaaccaagactctatctcttccaagttgaaggaatgttgtgaaaagccattgttggaaaagtctcactgtattgctgaagttgaaaacgatgaaatgccagctgacttgccatctttggctgctgacttcgttgaatctaaggacgtttgtaagaactacgctgaagctaaggacgtcttcttgggtatgttcttgtacgaatacgctagaagacacccagactactccgttgtcttgttgttgagattggctaagacctacgaaactaccttggaaaagtgttgtgctgctgctgacccacacgaatgttacgctaaggttttcgatgaattcaagccattggtcgaagaaccacaaaacttgatcaagcaaaactgtgaattgttcgaacaattgggtgaatacaagttccaaaacgctttgttggttagatacactaagaaggtcccacaagtctccaccccaactttggttgaagtctctagaaacttgggtaaggtcggttctaagtgttgtaagcacccagaagctaagagaatgccatgtgctgaagattacttgtccgtcgttttgaaccaattgtgtgttttgcacgaaaagaccccagtctctgatagagtcaccaagtgttgtactgaatctttggttaacagaagaccatgtttctctgctttggaagtcgacgaaacttacgttccaaaggaattcaacgctgaaactttcaccttccacgctgatatctgtaccttgtccgaaaaggaaagacaaattaagaagcaaactgctttggttgaattggtcaagcacaagccaaaggctactaaggaacaattgaaggctgtcatggatgatttcgctgctttcgttgaaaagtgttgtaaggctgatgataaggaaacttgtttcgctgaagaaggtaagaagttggtcgctgcttcccaagctgctttgggtttg

SEQ ID NO: 31 - protein, 792 aa, Artificial Sequence
  Description of Artificial Sequence Amino acid sequence of the N terminal endostatin albumin fusion protein
MKWVFIVSILFLFSSAYSRSLDKRHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASKDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 32 - DNA, 2376 bp, Artificial Sequence
  Description of Artificial Sequence DNA sequence of the C terminal albumin-endostatin fusion open reading frame
atgaagtgggtaagctttatttcccttctttttctctttagctcggcttattccaggagcttggataaaagagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttacacagccaccgcgacttccagccggtgctccacctggttgcgctcaacagccccctgtcaggcggcatgcggggcatccgcggggccgacttccagtgcttccagcaggcgcgggccgtggggctggcgggcaccttccgcgccttcctgtcctcgcgcctgcaggacctgtacagcatcgtgcgccgtgccgaccgcgcagccgtgcccatcgtcaacctcaaggacgagctgctgtttcccagctgggaggctctgttctcaggctctgagggtccgctgaagcccggggcacgcatcttctcctttgacggcaaggacgtcctgaggcaccccacctggccccagaagagcgtgtggcatggctcggaccccaacgggcgcaggctgaccgagagctactgtgagacgtggcggacggaggctccctcggccacgggccaggcctcctcgctgctggggggcaggctcctggggcagagtgccgcgagctgccatcacgcctacatcgtgctctgcattgagaacagcttcatgactgcctccaag

SEQ ID NO: 33 - protein, 792 aa, Artificial Sequence
  Description of Artificial Sequence Amino acid sequence of the C terminal albumin-endostatin fusion protein
MKWVSFISLLFLFSSAYSRSLDKRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 34 - DNA, 2607 bp, Artificial Sequence
  Description of Artificial Sequence DNA sequence of the N-terminal angiostatin (non glycosylated) - albumin fusion open reading frame
atgaagtgggttttcatcgtctccattttgttcttgttctcctctgcttactctagatctttggataagagagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgtgacatcccccgctgcacaacacctccaccatcttctggtcccacctaccagtgtctgaagggaacaggtgaaaactatcgcgggaatgtggctgttaccgtgtccgggcacacctgtcagcactggagtgcacagacccctcacacacatcaaaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtagacgctcacaagtccgaagtcgctcatcgattcaaggacctaggtgaggaaaacttcaaggctttggtcttgatcgctttcgctcaatacttgcaacaatgtccattcgaagatcacgtcaagttggtcaacgaagttaccgaattcgctaagacttgtgttgctgacgaatctgctgaaaactgtgacaagtccttgcacaccttgttcggtgataagttgtgtactgttgctaccttgagagaaacctacggtgaaatggctgactgttgtgctaagcaagaaccagaaagaaacgaatgtttcttgcaacacaaggacgacaacccaaacttgccaagattggttagaccagaagttgacgtcatgtgtactgctttccacgacaacgaagaaaccttcttgaagaagtacttgtacgaaattgctagaagacacccatacttctacgctccagaattgttgttcttcgctaagagatacaaggctgctttcaccgaatgttgtcaagctgctgataaggctgcttgtttgttgccaaagttggatgaattgagagacgaaggtaaggcttcttccgctaagcaaagattgaagtgtgcttccttgcaaaagttcggtgaaagagctttcaaggcttgggctgtcgctagattgtctcaaagattcccaaaggctgaattcgctgaagtttctaagttggttactgacttgactaaggttcacactgaatgttgtcacggtgacttgttggaatgtgctgatgacagagctgacttggctaagtacatctgtgaaaaccaagactctatctcttccaagttgaaggaatgttgtgaaaagccattgttggaaaagtctcactgtattgctgaagttgaaaacgatgaaatgccagctgacttgccatctttggctgctgacttcgttgaatctaaggacgtttgtaagaactacgctgaagctaaggacgtcttcttgggtatgttcttgtacgaatacgctagaagacacccagactactccgttgtcttgttgttgagattggctaagacctacgaaactaccttggaaaagtgttgtgctgctgctgacccacacgaatgttacgctaaggttttcgatgaattcaagccattggtcgaagaaccacaaaacttgatcaagcaaaactgtgaattgttcgaacaattgggtgaatacaagttccaaaacgctttgttggttagatacactaagaaggtcccacaagtctccaccccaactttggttgaagtctctagaaacttgggtaaggtcggttctaagtgttgtaagcacccagaagctaagagaatgccatgtgctgaagattacttgtccgtcgttttgaaccaattgtgtgttttgcacgaaaagaccccagtctctgatagagtcaccaagtgttgtactgaatctttggttaacagaagaccatgtttctctgctttggaagtcgacgaaacttacgttccaaaggaattcaacgctgaaactttcaccttccacgctgatatctgtaccttgtccgaaaaggaaagacaaattaagaagcaaactgctttggttgaattggtcaagcacaagccaaaggctactaaggaacaattgaaggctgtcatggatgatttcgctgctttcgttgaaaagtgttgtaaggctgatgataaggaaacttgtttcgctgaagaaggtaagaagttggtcgctgcttcccaagctgctttgggtttg

SEQ ID NO: 35 - protein, 869 aa, Artificial Sequence [sequence omitted]

SEQ ID NO: 36 - DNA, 2607 bp, Artificial Sequence
  Description of Artificial Sequence DNA sequence of the C-terminal albumin angiostatin (non glycosylated) - fusion open reading frame
atgaagtgggtaagctttatttcccttctttttctctttagctcggcttattccaggagcttggataaaagagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgtgacatcccccgctgcacaacacctccaccatcttctggtcccacctaccagtgtctgaagggaacaggtgaaaactatcgcgggaatgtggctgttaccgtgtccgggcacacctgtcagcactggagtgcacagacccctcacacacatcaaaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagta

SEQ ID NO: 37 - protein, 869 aa, Artificial Sequence
  Description of Artificial Sequence Amino acid sequence at the C-terminal albumin angiostatin (non glycosylated) - fusion protein
MKWVSFISLLFLFSSAYSRSLDKRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHQRTPENFPCKNLDENYCRNPDGKRAPWCHTTNSQVRWEYCKIPSCDSSPV

SEQ ID NO: 38 - DNA, 2109 bp, Artificial Sequence
  Description of Artificial Sequence DNA sequence of the N-terminal Kringle5- (GGS) 4GG-albumin fusion open reading frame
atgaagtgggttttcatcgtctccattttgttcttgttctcctctgcttactctagatctttggataagagatgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtggtggatccggtggttccggtggttctggtggttccggtggtgacgctcacaagtccgaagtcgctcaccggttcaaggacctaggtgaggaaaacttcaaggctttggtcttgatcgctttcgctcaatacttgcaacaatgtccattcgaagatcacgtcaagttggtcaacgaagttaccgaattcgctaagacttgtgttgctgacgaatctgctgaaaactgtgacaagtccttgcacaccttgttcggtgataagttgtgtactgttgctaccttgagagaaacctacggtgaaatggctgactgttgtgctaagcaagaaccagaaagaaacgaatgtttcttgcaacacaaggacgacaacccaaacttgccaagattggttagaccagaagttgacgtcatgtgtactgctttccacgacaacgaagaaaccttcttgaagaagtacttgtacgaaattgctagaagacacccatacttctacgctccagaattgttgttcttcgctaagagatacaaggctgctttcaccgaatgttgtcaagctgctgataaggctgcttgtttgttgccaaagttggatgaattgagagacgaaggtaaggcttcttccgctaagcaaagattgaagtgtgcttccttgcaaaagttcggtgaaagagctttcaaggcttgggctgtcgctagattgtctcaaagattcccaaaggctgaattcgctgaagtttctaagttggttactgacttgactaaggttcacactgaatgttgtcacggtgacttgttggaatgtgctgatgacagagctgacttggctaagtacatctgtgaaaaccaagactctatctcttccaagttgaaggaatgttgtgaaaagccattgttggaaaagtctcactgtattgctgaagttgaaaacgatgaaatgccagctgacttgccatctttggctgctgacttcgttgaatctaaggacgtttgtaagaactacgctgaagctaaggacgtcttcttgggtatgttcttgtacgaatacgctagaagacacccagactactccgttgtcttgttgttgagattggctaagacctacgaaactaccttggaaaagtgttgtgctgctgctgacccacacgaatgttacgctaaggttttcgatgaattcaagccattggtcgaagaaccacaaaacttgatcaagcaaaactgtgaattgttcgaacaattgggtgaatacaagttccaaaacgctttgttggttagatacactaagaaggtcccacaagtctccaccccaactttggttgaagtctctagaaacttgggtaaggtcggttctaagtgttgtaagcacccagaagctaagagaatgccatgtgctgaagattacttgtccgtcgttttgaaccaattgtgtgttttgcacgaaaagaccccagtctctgatagagtcaccaagtgttgtactgaatctttggttaacagaagaccatgtttctctgctttggaagtcgacgaaacttacgttccaaaggaattcaacgctgaaactttcaccttccacgctgatatctgtaccttgtccgaaaaggaaagacaaattaagaagcaaactgctttggttgaattggtcaagcacaagccaaaggctactaaggaacaattgaaggctgtcatggatgatttcgctgctttcgttgaaaagtgttgtaaggctgatgataaggaaacttgtttcgctgaagaaggtaagaagttggtcgctgcttcccaagctgctttgggtttg

SEQ ID NO: 39 - protein, 703 aa, Artificial Sequence
  Description of Artificial Sequence Amino acid sequence of the N-terminal Kringle5- (GGS) 4GG-albumin fusion protein
MKWVFIVSILFLFSSAYSRSLDKRCMFGNGKGYRGKRATTVTGTPCQDWAAQEPHRHSIFTPETNPRAGLEKNYCRNPDGDVGGPWCYTTNPRKLYDYCDVPQCGGSGGSGGSGGSGGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 40 - DNA, 2109 bp, Artificial Sequence
  Description of Artificial Sequence DNA sequence of the C-terminal albumin- (GGS) 4GG-Kringle5 fusion open reading frame
atgaagtgggtaagctttatttcccttctttttctctttagctcggcttattccaggagcttggataaaagagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggtaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaggtggttctggtggttccggtggttctggtggatccggtggttgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgt

SEQ ID NO: 41 - protein, 703 aa, Artificial Sequence
  Description of Artificial Sequence Amino acid sequence of the C-terminal albumin- (GGS) 4GG-Kringle5 fusion protein
MKWVSFISLLFLFSSAYSRSLDKRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGSGGSGGSGGSGGCMFGNGKGYRGKRATTVTGTPCQDWAAQEPHRHSIFTPETNPRAGLEKNYCRNPDGDVGGPWCYTTNPRKLYDYCDVPQC

SEQ ID NO: 42 - DNA, 22 bp, Artificial Sequence
  Description of Artificial Sequence Primer
tgtatgtttgggaatgggaaag

SEQ ID NO: 43 - DNA, 22 bp, Artificial Sequence
  Description of Artificial Sequence Primer
acactgagggacatcacagtag

SEQ ID NO: 44 - DNA, 37 bp, Artificial Sequence
  Description of Artificial Sequence Primer
gtgggatccggtggttgtatgtttgggaatgggaaag

SEQ ID NO: 45 - DNA, 35 bp, Artificial Sequence
  Description of Artificial Sequence Primer
cacaagcttattaacactgagggacatcacagtag

SEQ ID NO: 46 - DNA, 31 bp, Artificial Sequence
  Description of Artificial Sequence Primer
gtgagatcttgtatgtttgggaatgggaaag

SEQ ID NO: 47 - DNA, 34 bp, Artificial Sequence
  Description of Artificial Sequence Primer
cacggatccaccacactgagggacatcacagtag